SEQUENCE LISTING (16 sequences)

SEQ ID NO: 1 - protein, 5 aa, synthetic construct
SYWVD

SEQ ID NO: 2 - protein, 17 aa, synthetic construct
NIYPSDSETHYNQKFKD

SEQ ID NO: 3 - protein, 11 aa, synthetic construct
SYGYYGTWFAY

SEQ ID NO: 4 - protein, 15 aa, synthetic construct
RASESVDSSGNSFMH

SEQ ID NO: 5 - protein, 7 aa, synthetic construct
RASNLES

SEQ ID NO: 6 - protein, 9 aa, synthetic construct
QQSNEDPWT

SEQ ID NO: 7 - protein, 120 aa, synthetic construct
QVQLVQSGAEVKKPGASVKVSCKASGYTFTSYWVDWVRQAPGQGLEWMGNIYPSDSETHYNQKFKDRVTMTRDTSTSTVYMELSSLRSEDTAVYYCARSYGYYGTWFAYWGQGTLVTVSS

SEQ ID NO: 8 - protein, 111 aa, synthetic construct
DIVMTQSPDSLAVSLGERATINCRASESVDSSGNSFMHWYQQKPGQPPKLLIYRASNLESGVPDRFSGSGSGTDFTLTISSLQAEDVAVYYCQQSNEDPWTFGGGTKLEIK

SEQ ID NO: 9 - protein, 120 aa, synthetic construct
QVQLVQSGAEVKKPGASVKVSCKASGYTFTSYWVDWVRQAPGQGLEWIGNIYPSDSETHYNQKFKDRVTMTVDTSTSTVYMELSSLRSEDTAVYYCARSYGYYGTWFAYWGQGTLVTVSS

SEQ ID NO: 10 - protein, 120 aa, synthetic construct
QVQLVQSGAEVKKPGASVKVSCKASGYTFTSYWVDWVRQAPGQGLEWIGNIYPSDSETHYNQKFKDRVTMTVDTSTSTAYMELSSLRSEDSAVYYCARSYGYYGTWFAYWGQGTLVTVSS

SEQ ID NO: 11 - protein, 111 aa, synthetic construct
DIVMTQSPDSLAVSLGERATINCRASESVDSSGNSFMHWYQQKPGQPPKLLIYRASNLESGIPDRFSGSGSGTDFTLTISSVQAEDVAVYYCQQSNEDPWTFGGGTKLEIK

SEQ ID NO: 12 - protein, 111 aa, synthetic construct
DIVMTQSPDSLAVSLGERATINCRASESVDSSGNSFMHWYQQKPGQPPKLLIYRASNLESGIPDRFSGSGSGTDFTLTISSLQAEDVAVYYCQQSNEDPWTFGGGTKLEIK

SEQ ID NO: 13 - protein, 450 aa, synthetic construct
QVQLVQSGAEVKKPGASVKVSCKASGYTFTSYWVDWVRQAPGQGLEWMGNIYPSDSETHYNQKFKDRVTMTRDTSTSTVYMELSSLRSEDTAVYYCARSYGYYGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 218 aa, synthetic construct
DIVMTQSPDSLAVSLGERATINCRASESVDSSGNSFMHWYQQKPGQPPKLLIYRASNLESGVPDRFSGSGSGTDFTLTISSLQAEDVAVYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 1413 bp, synthetic construct
  source 1..1413: mol_type = other DNA
atgggatggtcatgtatcatcctttttctggtagcaactgcaactggagtacatagccaggtgcagttggtacaatcgggcgccgaagtgaagaaaccaggcgccagcgtcaaggtctcttgtaaagcatctggatataccttcacctcctattgggtcgattgggtccgccaagccccgggacagggcctggagtggatggggaacatttatccaagtgactctgaaactcactacaatcagaagttcaaggacagagtcaccatgacccgagatacaagtacaagcacagtttatatggagctgagtagcctgcgatcagaggacacagcagtctattactgcgctcggagctacggatactacggtacttggtttgcttactggggccagggcaccttggtgacagtgtcctctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagcgggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaataatag

SEQ ID NO: 16 - DNA, 720 bp, synthetic construct
  source 1..720: mol_type = other DNA
atggagacagacacactcctgctatgggtactgctgctctgggttccaggctccaccggcgatatcgtgatgacacaatctcctgactccctggccgtcagcctgggagaacgggccacaattaattgtcgtgcctctgagagcgtggactctagcggcaactctttcatgcactggtatcagcaaaaaccaggacagccacctaagttgctgatctaccgggctagcaacctcgaatccggagtgcccgatcggtttagtggcagtggcagtggcactgactttactctgaccatctcctcgcttcaagccgaggatgtggctgtgtattattgtcaacaatccaatgaggatccttggacctttggcggtggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatag

(19) United States
(12) Patent Application Publication
(10) Pub. No.: US 2025/0282882 A1
(43) Pub. Date: Sep. 11, 2025

(54) PHARMACEUTICAL FORMULATION COMPRISING ANTI-OX40 MONOCLONAL ANTIBODY

(71) Applicant: INMAGENE PTE. LTD., Singapore (SG)

(72) Inventors: Qiang SUN, Zhejiang, Hangzhou (CN); Xionghua JIN, Zhejiang, Taizhou (CN); Pengcheng FAN, Zhejiang, Hangzhou (CN); Zhihao XU, Zhejiang, Hangzhou (CN)

(21) Appl. No.: 18/701,246

(22) PCT Filed: Oct. 13, 2022
(86) PCT No.: PCT/CN2022/125017

§ 371 Date: Apr. 14, 2024

(30) Foreign Application Priority Data
Oct. 15, 2021 (WO) PCT/CN2021/124200
Sep. 9, 2022 (WO) PCT/CN2022/118065

(51) Int. Cl. C07K 16/28; A61K 39/00; A61K 47/18; A61K 47/22; A61K 47/26

(52) U.S. Cl. CPC C07K 16/2878; A61K 47/183; A61K 47/22; A61K 47/26; A61K 2039/505; A61K 2039/54

(57) ABSTRACT

Disclosed is a pharmaceutical formulation including a monoclonal OX40 antibody, a buffer, a stabilizer, and a surfactant, which can maintain the stability under various scenarios, such as manufacturing, packaging, sub-packaging, shipping, administration, and/or storage. Also disclosed are the use of the pharmaceutical formulation in the preparation of a drug for treating OX40-associated diseases, in particular, inflammatory and/or autoimmune diseases, and a method for preparing the pharmaceutical formulation.

### FIELD OF THE INVENTION

The present disclosure relates to pharmaceutical formulations, in particular the stable pharmaceutical formulations comprising a monoclonal anti-OX40 antibody or antigen-binding fragment thereof.

### BACKGROUND

Antibodies against human OX40 receptor (OX40) have been developed for treating various diseases, such as autoimmune diseases, inflammatory diseases, or other disorders, such as cancer. However, it has been found that these antibodies are not stable enough and are often subject to various chemical and physical degradation. Especially, the high-order structures of the antibodies are very fragile and prone to structural changes, such as denaturation, aggregation, and precipitation.

Denaturation refers to the changes in antibody's physical, chemical, and/or biological properties, which has been implicated in increasing the immunogenic potential of the antibodies. Protein aggregation occurs when protein molecule self-associates with one or more additional protein molecules, which often results in reduced bioactivity that affects drug potency, as well as increased possibility of immunological or antigenic reactions in patients. Precipitation occurs when, for example, pH or hydrophobicity changes, leading to the alteration of the interactions between the protein molecule and the aqueous environment or the disruption of the intramolecular interactions of the functional groups of the protein molecules through binding of salts or metals. Such degraded or unstable products, as well as aggregation or precipitation can have a great negative impact on the biological activity and the safety of the biological products. For example, aggregation, either protein aggregates or mixed aggregates of the therapeutic protein with an inactive excipient contained in the pharmaceutical formulation, may lead to immunogenic reactions, see Schellekens, H., Nat. Rev. Drug Discov. 1:457-62(2002); and Hesmeling, et al., Pharm. Res. 22:1997-2006 (2005).

Therefore, there is a need for novel pharmaceutical formulations of antibodies, such as monoclonal anti-OX40 antibodies, with increased stability and reduced toxicity of the pharmaceutical formulations.

### BRIEF SUMMARY OF THE INVENTION

The present disclosure provides stable pharmaceutical formulations comprising a monoclonal anti-OX40 antibody, which remain uniform and stable over a long period.

In one aspect, the present disclosure provides a pharmaceutical formulation comprising a monoclonal anti-OX40 antibody or antigen-binding fragment thereof, a buffer, a stabilizer, and a surfactant.

In certain embodiments, the monoclonal anti-OX40 antibody comprises a heavy chain and a light chain, wherein the heavy chain comprises a heavy chain variable region V_{H} which comprises:
HCDR1 comprising the amino acid sequence as set forth in SEQ ID NO: 1;
HCDR2 comprising the amino acid sequence as set forth in SEQ ID NO: 2,
HCDR3 comprising the amino acid sequence as set forth in SEQ ID NO: 3;
wherein the light chain comprises a light chain variable region V_{L} which comprises:
LCDR1 comprising the amino acid sequence as set forth in SEQ ID NO: 4,
LCDR2 comprising the amino acid sequence as set forth in SEQ ID NO: 5,
LCDR3 comprising the amino acid sequence as set forth in SEQ ID NO: 6; and
wherein the heavy chain further comprises Fc region variant, and the Fc region variant is human IgG1 N297A.

In certain embodiments, the heavy chain variable region V_{H} comprises the amino acid sequence selected from the group consisting of: SEQ ID NO: 7, SEQ ID NO: 9, and SEQ ID NO: 10.

In certain embodiments, the light chain variable region V_{L} comprises the amino acid sequence selected from the group consisting of: SEQ ID NO: 8, SEQ ID NO: 11, and SEQ ID NO: 12.

In certain embodiments, the concentration of the monoclonal anti-OX40 antibody in the pharmaceutical formulation is about 0.5-200 mg/ml, preferably about 40-60 mg/ml.

In certain embodiments, the pharmaceutical formulation has a pH of about 5.0-8.0.

In certain embodiments, the buffer is selected from the group consisting of acetate buffer, histidine buffer, citrate buffer, glutamic acid buffer, arginine buffer, citrate & arginine buffer, and glutamic acid & histidine buffer, aspartic acid & histidine buffer, wherein the concentration of the buffer in the pharmaceutical formulation is about 1-100 mmol/L.

In certain embodiments, the stabilizer is selected from the group consisting of sucrose, sorbitol, trehalose, xylitol and mannose, wherein the concentration of the stabilizer in the pharmaceutical formulation is about 0.5%-50% w/v.

In certain embodiments, the surfactant is selected from the group consisting of polysorbate 80 and polysorbate 20, wherein the concentration of the surfactant in the pharmaceutical formulation is about 0.001-0.1% w/v.

In certain embodiments, the concentration of the monoclonal anti-OX40 antibody in the pharmaceutical formulation is about 40-60 mg/ml, the concentration of the buffer in the pharmaceutical formulation is about 10-30 mmol/L, the concentration of the stabilizer in the pharmaceutical formulation is about 4-12% w/v, the concentration of the surfactant in the pharmaceutical formulation is about 0.01-0.05% w/v, and/or the pharmaceutical formulation has a pH of about 5.0-6.0.

In certain embodiments, the concentration of the monoclonal anti-OX40 antibody in the pharmaceutical formulation is about 50 mg/ml, the concentration of the buffer in the pharmaceutical formulation is about 20 mmol/L, the concentration of the stabilizer in the pharmaceutical formulation is about 4.5-8.8% w/v, the concentration of the surfactant in the pharmaceutical formulation is about 0.02-0.04% w/v, and/or wherein the pharmaceutical formulation has a pH of about 5.0-5.5.

In certain embodiments, the concentration of the stabilizer in the pharmaceutical formulation is about 8% w/v, the concentration of the surfactant in the pharmaceutical formulation is about 0.02% w/v, and/or the pharmaceutical formulation has a pH of about 5.0.

In certain embodiments, the buffer is glutamic acid & histidine buffer, aspartic acid & histidine buffer or combination thereof, the stabilizer is sucrose, sorbitol, trehalose, or combination thereof, and/or the surfactant is polysorbate 80.

In certain embodiments, the pharmaceutical formulation provided herein comprises:
a monoclonal anti-OX40 antibody or antigen-binding fragment thereof, at a concentration of about 40-60 mg/ml,
glutamic acid & histidine buffer or aspartic acid & histidine buffer at a concentration of about 10-30 mmol/L,
sucrose at a concentration of about 4-12% w/v, and
polysorbate 80 at a concentration of about 0.01-0.05% w/v,
and the pharmaceutical formulation has a pH of about 5.0-5.5.

In certain embodiments, the pharmaceutical formulation provided herein comprises:
a monoclonal anti-OX40 antibody or antigen-binding fragment thereof, at a concentration of about 50 mg/ml,
glutamic acid & histidine buffer or aspartic acid & histidine buffer at a concentration of about 20 mmol/L,
sucrose at a concentration of about 8% w/v, and
polysorbate 80 at a concentration of about 0.02% w/v,
and wherein the pharmaceutical formulation has a pH of about 5.0.

In certain embodiments, the pharmaceutical formulation is suitable for subcutaneous administration or for intravenous administration.

In another aspect, the present disclosure also provides use of the pharmaceutical formulation provided herein in the manufacture of a medicament for the treatment or prevention of OX40-associated disease.

In certain embodiments, the OX40-associated disease is inflammation and/or autoimmune diseases, such as graft-versus-host disease.

In another aspect, the present disclosure also provides a method of treating OX40-associated disease in a subject in need thereof, comprising administering a therapeutically effective amount of the pharmaceutical formulation provided herein to the subject.

In certain embodiments, the administration is via subcutaneous injection or intravenous injection.

In another aspect, the present disclosure also provides a method of preparing the pharmaceutical formulation provided herein, comprising combining the buffer, the stabilizer, the surfactant, and a pharmaceutically effective amount of the monoclonal anti-OX40 antibody or antigen-binding fragment thereof.

In another aspect, the present disclosure also provides a kit comprising the pharmaceutical formulation provided herein in one or more containers.

In certain embodiments, the kit provided herein further comprises instructions for use of the kit.

### BRIEF DESCRIPTION OF FIGURES

FIG. 1 shows the result of protein concentration in solubility profiling study.

FIG. 2 shows the overlay of DSC in pH/buffer screening.

FIG. 3 shows the trend of the main peak by SEC-HPLC.

FIG. 4 shows the trend of HMW by SEC-HPLC.

FIG. 5 shows the trend of LMW by SEC-HPLC.

FIG. 6 shows the trend of the main peak by iCIEF.

FIG. 7 shows the trend of the acidic peak by iCIEF.

FIG. 8 shows the trend of the basic peak by iCIEF.

FIG. 9 shows the trend of the purity by Caliper-NR.

FIG. 10 shows the trend of the purity by Caliper-R.

FIG. 11 shows the overlay of DSC.

FIG. 12 shows the trend of the main peak by SEC-HPLC.

FIG. 13 shows the trend of the HMW by SEC-HPLC.

FIG. 14 shows the trend of the LMW by SEC-HPLC.

FIG. 15 shows the trend of the main peak by iCIEF.

FIG. 16 shows the trend of the acidic peak by iCIEF.

FIG. 17 shows the trend of the basic peak by iCIEF.

FIG. 18 shows the trend of the purity by Caliper-SDS-NR.

FIG. 19 shows the trend of the purity by Caliper-SDS-R.

### DETAILED DESCRIPTION OF THE INVENTION

The following description of the disclosure is merely intended to illustrate various embodiments of the present disclosure. The specific examples described should not be construed to limit the scope of the present disclosure. Various equivalents, variations, and modifications may be made by those of ordinary skilled in the art without departing from the spirit and scope of the present disclosure, and it is understood that the equivalents are also encompassed herein. All references cited herein, including publications, patents and patent applications are incorporated herein by reference in their entirety.

### Definitions

As used herein, the term “a”, “an” or “the” refers to both singular and plural unless the context clearly dictates otherwise.

Reference to “about” a value or parameter herein includes (and describes) embodiments that are directed to that value or parameter per se. For example, description referring to “about X” includes description of “X”. Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of cell and tissue culture, molecular biology, and protein and oligo- or polynucleotide chemistry, laboratory procedures and techniques of analytical chemistry, synthetic organic chemistry, and medicinal and pharmaceutical chemistry described herein are those well-known and commonly used in the art.

The term “pharmaceutical formulation” as used herein refers to a combination of one or more active pharmaceutical ingredients (APIs) with at least one other ingredient for, e.g., further processing (e.g., lyophilization, reconstitution, titration, dilution), storage, sale, and/or administration by a specific route at a specific dosage to treat a specific disease.

The term “active pharmaceutical ingredient” or “API”, as used herein, refers to a macromolecule such as a polypeptide, nucleic acid, lipid, or carbohydrate, or building block thereof, which can be used as therapeutics, such as a therapeutic antibody (e.g., monoclonal anti-OX40 antibody) or antigen-binding fragment thereof.

When describing a range of values, it is to be understood that the features being described can be an individual value within the range. For example, “a pH of about 5.0-8.0” can be, without limitation, pH 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, etc., as well as any value in between the above values. The term “a pH of about 5.0-8.0” should not be construed as a pH of a pharmaceutical formulation that varies 3 pH units in the range from pH 5.0 to pH 8.0 during manufacturing, packaging, sub-packaging, shipping, administration and/or storage; instead, the term “a pH of about 5.0-8.0” means that a value can be picked in the range of about 5.0-8.0 for the pH of the solution, and the pH is buffered at about the picked pH value during manufacturing, packaging, sub-packaging, shipping, administration and/or storage.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “subject” includes human and non-human animals. Non-human animals include all vertebrates, e.g., mammals and non-mammals, such as non-human primates, mouse, rat, cat, rabbit, sheep, dog, cow, chickens, amphibians, and reptiles. Unless otherwise indicated, the terms “patient” or “subject” are used herein interchangeably.

### Formulation

This present disclosure provides novel pharmaceutical formulations that retain increased stability of an API under a variety of different manufacturing, packaging, sub-packaging, shipping, administration, and storage conditions. The pharmaceutical formulations of the present disclosure also exhibit reduced toxicity and increased therapeutic efficacy. APIs used with the pharmaceutical formulations provided herein may comprise, inter alia, therapeutic antibodies, such as monoclonal anti-OX40 antibody or antigen-binding fragment thereof.

In one aspect, the present disclosure provides a pharmaceutical formulation, which comprises a monoclonal anti-OX40 antibody or antigen-binding fragment thereof, a buffer, a stabilizer, and a surfactant. In some embodiments, the pharmaceutical formulation of the present disclosure has a pH of about 5.0-8.0 (e.g., about 5.0-6.0, about 5.0-5.5, or about 5.0).

In a further aspect, the present disclosure provides a pharmaceutical formulation, which comprises a monoclonal anti-OX40 antibody or antigen-binding fragment thereof at a concentration of about 0.5-200 mg/ml (e.g., about 1-180 mg/ml, about 10-160 mg/ml, about 15-140 mg/ml, about 20-120 mg/ml, about 25-100 mg/ml, about 30-80 mg/ml, about 40-60 mg/ml, or about 50 mg/ml), a buffer (e.g., glutamic acid & histidine buffer, or aspartic acid & histidine buffer) at a concentration of about 1-100 mmol/L (e.g., about 10-90 mmol/L, about 10-80 mmol/L, about 10-70 mmol/L, about 10-60 mmol/L, about 10-50 mmol/L, about 10-40 mmol/L, about 10-30 mmol/L, or about 20 mmol/L), a stabilizer (e.g., sucrose, sorbitol, or trehalose) at a concentration of about 0.5%-50% w/v (e.g., about 1% to about 40% (w/v), about 2% to about 30% (w/v), about 3% to about 20% (w/v), about 3.2% to about 18% (w/v), about 3.4% to about 16% (w/v), about 3.6% to about 14% (w/v), about 4% to about 12% (w/v), about 6% to about 10% (w/v), or about 8% (w/v)), and a surfactant (e.g., polysorbate 80 (PS80) or polysorbate 20 (PS20)) at a concentration of about 0.001-0.1% (w/v) (e.g., about 0.002% to about 0.08% (w/v), about 0.004% to about 0.06% (w/v), about 0.006% to about 0.05% (w/v), about 0.008% to about 0.05% (w/v), about 0.01% to about 0.05% (w/v), or about 0.02% (w/v)), with a pH of about 5.0-8.0 (e.g., about 5.0-6.0, about 5.0-5.5, or about 5.0).

In another aspect, the present disclosure also provides a pharmaceutical formulation, which comprises a monoclonal anti-OX40 antibody or antigen-binding fragment thereof at a concentration of about 0.5-200 mg/ml (e.g., about 1-180 mg/ml, about 10-160 mg/ml, about 15-140 mg/ml, about 20-120 mg/ml, about 25-100 mg/ml, about 30-80 mg/ml, about 40-60 mg/ml, or about 50 mg/ml), a buffer (e.g., glutamic acid & histidine buffer, or aspartic acid & histidine buffer) at a concentration of about 10-30 mmol/L, a stabilizer (e.g., sucrose, sorbitol, or trehalose) at a concentration of about 4% to about 12% (w/v), about 0.5%-10% (w/v), or about 4%-14% (w/v), and a surfactant (e.g., polysorbate 80) at a concentration of about 0.01% to about 0.05% (w/v), with a pH of about 5.0-6.0.

In another aspect, the present disclosure also provides a pharmaceutical formulation, which comprises a monoclonal anti-OX40 antibody or antigen-binding fragment thereof at a concentration of about 0.5-200 mg/ml (e.g., about 1-180 mg/ml, about 10-160 mg/ml, about 15-140 mg/ml, about 20-120 mg/ml, about 25-100 mg/ml, about 30-80 mg/ml, about 40-60 mg/ml, or about 50 mg/ml), a buffer (e.g., glutamic acid & histidine buffer, or aspartic acid & histidine buffer) at a concentration of about 20 mmol/L, a stabilizer (e.g., sucrose, sorbitol, or trehalose) at a concentration of about 8% (w/v), about 4.5% (w/v), or about 8.8% (w/v), and a surfactant (e.g., polysorbate 80) at a concentration of about 0.01% to about 0.05% (w/v) (e.g., about 0.01% to about 0.04% (w/v), about 0.015% to about 0.035% (w/v), about 0.02% to about 0.03% (w/v), about 0.025% (w/v), or about 0.02% (w/v)), with a pH of about 5.0-6.0 (e.g., about 5.0-5.5 or about 5.0) to achieve sufficient stability.

The pharmaceutical formulations provided herein exhibit enhanced stability with improved resistance to changes, such as temperature, humidity, time and physical motion (e.g., agitation). As used herein, the term “stability” with respect to a pharmaceutical formulation refers to the optimal retention (which does not have to be 100%) of structure, function, and/or biological activity of an API (e.g., a monoclonal anti-OX40 antibody or antigen-binding fragment thereof) within the pharmaceutical formulation. As used herein, the term “retention of stability” with respect to a pharmaceutical formulation refers to relative value (expressed in percentage) of the stability of the pharmaceutical formulation after storage under certain conditions compared to the stability of the pharmaceutical formulation before such storage.

The stability of a pharmaceutical formulation may include physical stability, chemical stability and/or physicochemical stability of API.

Physical stability can be reflected by the percentage of protein monomers, which can be determined by measurements of the percentage of monomer before and after storage under certain conditions via, for example, size exclusion chromatography (SEC).

Chemical stability can be reflected by the level of chemical modifications, such as deamidation, pyroglutamate formation, and/or lysine truncation, which can be determined, by measurements of the charge heterogeneity before and after storage under certain conditions via, for example, imaged capillary isoelectric focusing (iCIEF) with cation-exchange chromatography (CEX) and/or anion-exchange chromatography (AEX) analysis. The iCIEF result may include main peaks, acidic peaks and basic peaks, along with the pI of the main peak. Acidic peaks stand for the acidic species, which are defined as the antibody variants that elute earlier than the main peak during cation-exchange chromatography (CEX) or later than the main peak during anion exchange chromatography (AEX) analysis. Acid species can be formed through modifications including sialic acid, deamidation, non-classical disulfide linkage, trisulfide bonds, high mannose, glycation, modification by maleuric acid, cysteinylation, reduced disulfide bonds, non-reduced species and/or fragments. Basic peaks stand for basic species, which are defined as the materials that elute later than the main peak during CEX and earlier than the main peak during AEX analyses. Basic species can be formed through modifications including C-terminal Lys, N-terminal Glu, Isomerization of Asp, Succinimide, Met oxidation, Amidation, Incomplete disulfide bonds, Incomplete removal of leader sequence, Mutation from Ser to Arg, glycosylation, Fragments and/or Aggregates. Main peak refers to the main species, which stands for the target antibody molecule that elutes as the major peak on chromatograms. The main species does not necessarily correspond to the unmodified or non-degraded antibody. In fact, the main peak typically consists of species of antibodies with three types of typical post-translational modifications: (1) cyclization of the N-terminal glutamine (Gln) to pyroGlu; (2) removal of the heavy chain C-terminal lysine (Lys); and (3) glycosylation of the conserved asparagine (Asn) residue in the C_{H2} domain with neutral oligosaccharides. Chemical stability can also be reflected by the purity of the API (e.g., truncation or fragmentation level) before and after storage under certain conditions via, for example, Caliper-SDS and SEC.

Physicochemical stability of API can be reflected by the level of low molecular weight percentage (LMW %) and/or the level of high molecular weight percentage (HMW %). As used herein, the term “low molecular weight percentage”, used interchangeably with the term “LMW %”, refers to the percentage of low molecular weight (LMW) impurities (e.g., Fab, Fc and single chain), which can occur through several pathways, such as hydrolysis, free radical induced fragmentation, and enzymatic cleavage, and indicates the physicochemical instability during manufacturing, storage, transportation and administration. As used herein, the term “high molecular weight percentage”, used interchangeably with the term “HMW %”, refers to the percentage of high molecular weight (HMW) impurities (e.g., dimers, trimers and multimers), which are formed through various mechanisms, such as molecular interactions and chemical cross-linking and indicates the colloidal and conformational instability during manufacturing, storage, transportation and administration. LMW % and HMW % can be determined, by measurements before and after storage under certain conditions via, for example, SEC.

The stability of a pharmaceutical formulation can also include thermal stability, which can be reflected by the temperature under which the protein starts to unfold (i.e., Tmonset), and/or by the temperature at which the first/second protein domain is half unfolded (i.e., Tm1/Tm2), as measured by differential scanning calorimetry (DSC).

The stability of a pharmaceutical formulation can also include thermodynamic stability of API, which can be reflected by Tagg and/or kD, as measured by dynamic light scattering (DLS). DLS provides information of the hydrodynamic size and size distribution of particles (e.g., anti-OX40 antibody or antigen-binding fragment thereof) in solution, which is commonly examined as a function of time and temperature. The temperature at which protein molecules (e.g., anti-OX40 antibody or antigen-binding fragment thereof) start to show a tendency to oligomerize or aggregate is named as aggregation temperature (Tagg). Tagg depends on the buffer composition. The higher Tagg is, the more stable the protein (e.g., anti-OX40 antibody or antigen-binding fragment thereof) is, and the longer shelf-life the protein would have. The information provided by DLS can also be analyzed to determine the translational diffusion coefficient, which is a function of concentration, and the analysis of the translational diffusion coefficient versus concentration leads to the diffusion interaction parameter kD. A positive kD signifies repulsive interactions and a negative kD implies attractive intermolecular interactions. A positive kD value represents repulsive intermolecular interaction, while a negative kD value represents attractive intermolecular interaction. Thus, a larger positive kD value implies less tendency of aggregation.

In some embodiments, the concentration of antibody protein, purity of protein, activity of protein, pH of the formulation, osmotic pressure of the formulation, appearance of the formulation, insoluble particles in the formulation, etc. may serve as indicators of the stability of the pharmaceutical formulation. Various analytical techniques for measuring protein stability are available in the art and are reviewed in Peptide and Protein Drug Delivery, 247-301, edited by Vincent Lee, Marcel Dekker Inc., New York, New York Press (1991) and Jones, A. Adv. Drug Delivery Rev. 10: 29-90 (1993). In some embodiments, the stability of the pharmaceutical formulation can be measured by methods known in the art at a selected condition for a selected time period.

In some embodiments, the percentage of monomer remaining after storage (4 weeks at 40° C.) or repeated freeze-thawing (freeze-thaw for 5 cycles) or agitation (agitation at 25° C. for 3 days) of the API of the pharmaceutical formulation provided herein can be between about 80% and about 100%, between about 85% and about 99%, between about 90% and about 99%, or between about 95% and about 99%, as measured by SEC-HPLC. Accordingly, the API within the pharmaceutical formulation of the present disclosure can retain at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or even 100% of physical stability as compared to that of the API at an initial time point, after storage (4 weeks at 40° C.) or repeated freeze-thawing (freeze-thaw for 5 cycles) or agitation (agitation at 25° C. for 3 days), as measured by SEC-HPLC.

In some embodiments, the LMW impurities (e.g., Fab, Fc and single chain) after storage (2 or 4 weeks at 40° C.) or repeated freeze-thawing (freeze-thaw for 3 cycles) or agitation (agitation at 25° C. for 3 days) of the API of the pharmaceutical formulation provided herein is between about 0.1% and about 3.4%, between about 0.15% and about 3.35%, between about 0.2% and about 3.3%, between about 0.3% and about 3.2%, between about 0.4% and about 3.1%, or between about 0.6% and about 2% as measured by SEC-HPLC. Accordingly, the API in the pharmaceutical formulation of the present disclosure can retain at least 96.6%, at least 97%, at least 98%, at least 99.0%, at least 99.1%, at least 99.2%, at least 99.3%, at least 99.4%, at least 99.5%, at least 99.6%, at least 99.7%, at least 99.8% or even at least 99.9% of physicochemical stability as compared to that of the API at an initial time point, after storage (2 or 4 weeks at 40° C.) or repeated freeze-thawing (freeze-thaw for 3 cycles) or agitation (agitation at 25° C. for 3 days), as measured by SEC-HPLC. In particular, the LMW impurities (e.g., Fab, Fc and single chain) after repeated freeze-thawing for 3 cycles or agitation at 25° C. for 3 days of the API of the pharmaceutical formulation provided herein is merely about 0.1% as measured by SEC-HPLC, and accordingly, the API in the pharmaceutical formulation of the present disclosure can retain about 99.9% of physicochemical stability as compared to that of the API at an initial time point, after repeated freeze-thawing for 3 cycles or agitation at 25° C. for 3 days as measured by SEC-HPLC.

In some embodiments, the purity after storage (4 weeks at 40° C.) or repeated freeze-thawing (freeze-thaw for 5 cycles) or agitation (agitation at 25° C. for 3 days) of the API of the pharmaceutical formulation provided herein can be between about 90% and about 99%, about 91% and about 99%, about 92% and about 99%, about 93% and about 99%, about 94% and about 99%, or between about 95% and about 99%, as measured by non-reduced Caliper-SDS. Accordingly, the API within the pharmaceutical formulation of the present disclosure can retain at least 88%, at least 89%, at least 90%, at least 91%, at least 92%, at least 93%, at least 94%, at least 95%, at least 96%, at least 97%, at least 98%, at least 99.0%, at least 99.1%, at least 99.2%, at least 99.3%, at least 99.4%, at least 99.5%, at least 99.6%, at least 99.7%, at least 99.8%, at least 99.9%, or even 100% of chemical stability as compared to that of the API at an initial time point, after storage (4 weeks at 40° C.) or repeated freeze-thawing (freeze-thaw for 5 cycles) or agitation (agitation at 25° C. for 3 days), as measured by non-reduced Caliper-SDS.

In some embodiments, the purity after storage (4 weeks at 40° C.) or repeated freeze-thawing (freeze-thaw for 5 cycles) or agitation (agitation at 25° C. for 3 days) of the API of the pharmaceutical formulation provided herein can be between about 90% and about 100%, about 91% and about 100%, about 92% and about 100%, about 93% and about 100%, about 94% and about 100%, or between about 95% and about 100%, as measured by reduced Caliper-SDS. Accordingly, the API within the pharmaceutical formulation of the present disclosure can retain at least 88%, at least 89%, at least 90%, at least 91%, at least 92%, at least 93%, at least 94%, at least 95%, at least 96%, at least 97%, at least 98%, at least 99.0%, at least 99.1%, at least 99.2%, at least 99.3%, at least 99.4%, at least 99.5%, at least 99.6%, at least 99.7%, at least 99.8%, at least 99.9%, or even 100% of chemical stability as compared to that of the API at an initial time point, after storage (4 weeks at 40° C.) or repeated freeze-thawing (freeze-thaw for 5 cycles) or agitation (agitation at 25° C. for 3 days), as measured by reduced Caliper-SDS.

In some embodiments, the Tmonset of the pharmaceutical formulation provided herein is no less than about 50° C., no less than about 50.5° C., no less than about 51° C., no less than about 51.5° C., no less than about 52° C., no less than about 52.5° C., or no less than about 53° C., as measured by DSC.

In certain embodiments, the Tm1 of the pharmaceutical formulation provided herein is no less than about 60° C., no less than about 60.1° C., no less than about 60.2° C., no less than about 60.3° C., no less than about 60.4° C., no less than about 60.5° C., no less than about 60.6° C., no less than about 60.7° C., no less than about 60.8° C., no less than about 60.9° C., no less than about 61° C., no less than about 61.2° C., no less than about 61.4° C., no less than about 61.6° C., no less than about 61.8° C., no less than about 62° C., no less than about 62.2° C., no less than about 62.4° C., no less than about 62.6° C., no less than about 62.8° C., no less than about 63° C., or no less than about 63.2° C., as measured by DSC.

In certain embodiments, the Tm2 of the pharmaceutical formulation provided herein is no less than about 75° C., no less than about 76° C., no less than about 76.1° C., no less than about 76.2° C., no less than about 76.3° C., no less than about 76.4° C., no less than about 76.5° C., no less than about 76.6° C., no less than about 76.7° C., no less than about 76.8° C., no less than about 76.9° C., no less than about 77° C., no less than about 77.2° C., no less than about 77.4° C., no less than about 77.6° C., no less than about 77.8° C., no less than about 78° C., no less than about 78.1° C., no less than about 78.2° C., or no less than about 78.3° C., as measured by DSC.

In certain embodiments, the kD of the pharmaceutical formulation provided herein is no less than about 10, no less than about 11, no less than about 12, no less than about 12.5, no less than about 13, no less than about 13.5, no less than about 14, no less than about 15, no less than about 16, no less than about 17, no less than about 18, or no less than about 19, as measured by DLS at 20° C. to 40° C.

In certain embodiments, the Tagg of the pharmaceutical formulation provided herein is no less than about 59° C., no less than about 59.2° C., no less than about 59.4° C., no less than about 59.6° C., no less than about 59.8° C., no less than about 60° C., no less than about 60.2° C., no less than about 60.4° C., no less than about 60.6° C., no less than about 60.8° C., no less than about 61° C., no less than about 61.2° C., or no less than about 61.4° C., as measured by DLS for formulation comprising the API at a concentration from 2 mg/mL to 10 mg/mL.

In some embodiments, the stability of pharmaceutical formulations provided herein can be measured by the appearance of the formulation after storage (e.g., at 40° C. for 1, 2 or 4 weeks), repeated freeze-thawing (e.g., freeze-thaw from −70° C. to room temperature for 3 or 5 cycles) or agitation (e.g., at 300 rpm, 25° C. for 1 or 3 days). In certain embodiments, no visible particles were observed in the pharmaceutical formulations of the present disclosure.

In some embodiments, the stability of pharmaceutical formulations provided herein can be measured by the pH of the formulation after storage (e.g., at 40° C. for 1, 2 or 4 weeks), repeated freeze-thawing (e.g., freeze-thaw from −70° C. to room temperature for 3 or 5 cycles) or agitation (e.g., at 300 rpm, 25° C. for 1 or 3 days). In certain embodiments, almost no changes in pH were observed for the pharmaceutical formulations of the present disclosure after storage, as compared to the formulation at an initial time point.

In some embodiments, the stability of pharmaceutical formulations provided herein can be measured by the API concentration of the formulation after storage (e.g., at 40° C. for 2 or 4 weeks), repeated freeze-thawing (e.g., freeze-thaw from −70° C. to room temperature for 3 or 5 cycles) or agitation (e.g., at 300 rpm, 25° C. for 1 or 3 days). In certain embodiments, the API concentration changes no more than 2%, no more than 1.9%, no more than 1.8%, no more than 1.7%, no more than 1.6%, no more than 1.5%, no more than 1.4%, no more than 1.3%, no more than 1.2%, no more than 1.1%, no more than 1.0%, no more than 0.9%, no more than 0.8%, no more than 0.7%, no more than 0.6%, no more than 0.5%, no more than 0.4%, no more than 0.3%, no more than 0.2%, no more than 0.1%, as compared to that of the formulation at an initial time point, as determined by UV280 readings using a spectrophotometer. In certain embodiments, no change in the API concentration was observed for the pharmaceutical formulations of the present disclosure.

In some embodiments, the stability of pharmaceutical formulations provided herein can be measured by the number of sub-visible particles in formulations after storage (e.g., at 40° C. for 4 weeks), repeated freeze-thawing (e.g., freeze-thaw from −70° C. to room temperature for 5 cycles) or agitation (e.g., at 300 rpm, 25° C. for 3 days), as shown, for example, in Example 4, Table 18. In particular, the number of sub-visible particles of in formulations after agitation (e.g., at 300 rpm, 25° C. for 3 days) is below 1500/mL, 1400/mL, 1300/mL, 1200/mL, 1100/mL, 1000/mL, 900/mL, 800/mL, 700/mL, 600/mL, 500/mL, 400/mL, 300/mL, 200/mL, 100/mL, 90/mL, 80/mL, 70/mL, 60/mL, 50/mL, 40/mL, 30/mL, 20/mL, 10/mL, or even below 2/mL. The sub-visible particles may induce anti-drug antibodies within a patient, which may negatively affect the therapeutic efficacy and/or elicit abnormal immune responses.

In some embodiments, the stability of the pharmaceutical formulation provided herein can be measured by activity (e.g., binding potency) of API. Activity of API can be measured using, for example, an in vitro, in vivo and/or in situ assay that is indicative of the API's function. Retention of stability of an API in the pharmaceutical formulation of the present disclosure can include, for example, retention of activity of the API between about 50% and about 100% or more, after storage at 40° C. for 4 weeks, depending on the variability of the assays. For example, the pharmaceutical formulation provided herein ca retain between about 80% and about 99%, between about 85% and about 99%, between about 86% and about 99%, between about 88% and about 99%, between about 90% and about 99%, between about 92% and about 99%, between about 94% and about 99%, between about 96% and about 99% or between about 98% and about 99% of activity as compared to that of the API at an initial time point, after storage or repeated freeze-thawing or agitation of the API of the pharmaceutical composition provided herein, as measured by binding assay.

In some embodiments, the retention of activity of the API of the pharmaceutical formulation of the present disclosure can be at least 90%, at least 91%, at least 92%, at least 93%, at least 94%, at least 95%, at least 96%, at least 97% or at least 99%. In some other embodiments, the retention of activity of the API of the pharmaceutical formulation of the present disclosure can be greater than 100%, for example, 102%, 104%, 106%, 108%, 110% or 112% or more after storage or repeated freeze-thawing or agitation of the API of the pharmaceutical composition as compared to the activity of the API at an initial time point.

The term “an initial time point”, as used herein, refers to the time that an API is first prepared in a pharmaceutical formulation or first examined for quality (for example, physical and/or chemical stability), which can be represented by TO.

In some embodiments, the pharmaceutical formulation of the present disclosure can maintain stable over a long period of time, wherein the stability and/or functional activity of the API as mentioned above are maintained relatively constant over time. The pharmaceutical formulation of the present disclosure may be subjected to tests of long-term stability, for example, the pharmaceutical formulation can be stored at 2-8° C. for 1 year, and samples are taken at the 1st, 3rd, 6th and 12th month for measurement. In certain embodiments, the pharmaceutical formulation of the present disclosure can maintain stable and functional for at least 1 month, at least 2 months, at least 3 months, at least 4 month, at least 5 months, at least 6 months, at least 7 month, at least 8 months, at least 9 months, at least 10 month, at least 11 months, or at least 12 months.

The pharmaceutical formulations of the present disclosure can be approved for pharmaceutical use by an international or national authority empowered by law to grant such approval for example, China's National Medical Products Administration (NMPA), United States Food and Drug Administration (FDA), the European Agency for the Evaluation of Medical Products (EMEA), Japan's Ministry of Health, Labor and Welfare (MHLW), Therapeutic Goods Administration (TGA), Taiwan Food and Drug Administration (TFDA), or their successor(s) in this authority, particularly preferably the NMPA or its successor(s) in this authority.

One of the advantages of the present disclosure is to provide stabilized pharmaceutical formulations against stresses that can occur during manufacturing, packaging, sub-packaging, shipping, administration and/or storage, with reduced toxicity and increased therapeutic efficacy. The stabilized pharmaceutical formulations provided herein may increase the ease of administration, reduce the frequency of administration, and reduce the amount of pain experienced by a patient upon injection. For example, administration via parenteral routes of intravenous or subcutaneous would be safer and more efficacious when the pharmaceutical formulation maintain physical, chemical, physicochemical, and/or thermal stability during manufacture, packaging, sub-packaging, shipping, storage and administration.

The stabilization of an API in a pharmaceutical composition against stresses occurred during manufacturing, packaging, sub-packaging, shipping, administration and storage are mainly conferred by various excipients of the pharmaceutical compositions. As used herein, the term “excipient” refers to a therapeutically inactive substance, such as a buffer, stabilizer, surfactant, tonicity agent, cryoprotectant, bulking agent, diluent, lyoprotectant, vehicle, metal ion source, anti-oxidant, preservative and/or chelating agent, which are well known in the art and description of which can be found in, for example, Wang W., Int. J. Pharm. 203:1-60 (2000) and Wang W., Int. J. Pharm. 185:129-88 (1999). The composition of the excipients in the pharmaceutical formulations provided herein minimized the extent of protein degradation/optimized the protein stability, and, consequently, retained the safety and efficacy of the API. The detailed description of the excipients used in the pharmaceutical formulations of the present disclosure follows.

### Buffer

Maintaining desired pH of a pharmaceutical formulation would positively affect the stability, effectiveness, and shelf life of the pharmaceutical formulation. To maintain pH, one or more buffering agents or buffers can be included in a pharmaceutical formulation. The term “buffer” refers to a buffered solution that resists changes in pH by the action of its acid-base conjugate components, which is known to be safe when used in a pharmaceutical formulation and maintains or controls the pH of the formulation in a desired range. Acceptable buffers capable of controlling the pH in a range from mild acidic pH to mild alkaline pH (e.g. pH 5.0-8.0) include, but are not limited to, one or any combination of phosphate buffer, acetate buffer, citrate buffer, arginine buffer, 2-amino-2-hydroxymethyl-1,3-propanediol (TRIS) buffer, histidine buffer, glutamic acid buffer, aspartic acid buffer, glutamic acid & histidine buffer, aspartic acid & histidine buffer, citrate & arginine buffer, and the like.

The pharmaceutical formulation of the present disclosure may comprise a buffer that allows the pharmaceutical formulation to have a pH of 5.0-8.0, such as a pH of 5.0-5.5, 5.5-6.5, or 6.5-8.0. In some embodiments, suitable buffers allow the pharmaceutical formulations of the present disclosure to have a pH of 5.0-6.0. In some embodiments, suitable buffers allow the pharmaceutical formulations of the present disclosure to have a pH of 5.0-5.5. In particular, the pH of the pharmaceutical formulation of the present disclosure may be any pH value in the pH ranges listed above, such as 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0. In preferred embodiments, suitable buffers allow the pharmaceutical formulations of the present disclosure to have a pH of about 5.0.

Examples of buffers that can control the pH of a pharmaceutical formulation within a desired range include acetate buffer, arginine buffer, glutamic acid buffer, aspartic acid buffer, histidine buffer, a citrate buffer, a phosphate buffer, and other organic or inorganic acid buffers. These buffers can be used alone, alternatively, two or more of these buffers may be used in combination.

The “glutamic acid buffer”, used interchangeably with the “glutamate buffer”, refers to a buffer comprising glutamic acid optionally in equilibrium with its respective conjugate base. The glutamic acid form of the glutamic acid buffer may comprise glutamic acid, glutamate ion and/or glutamate comprising glutamate salt, such as sodium, potassium, ammonium, calcium or magnesium salts of glutamate. This term includes both L and D forms of glutamic acid. The buffering capacity of the glutamate buffer is highly related to the pKa values of glutamic acid. It is well acknowledged that the buffering zones of an amino acid is the pH ranges near its pKa values. Glutamic acid has the pKa values of 2.2, 9.7, as well as a side chain pKa of 4.3, detailed description of the pKa values of amino acids can be seen in, for example, Amino Acids, the Henderson-Hasselbalch Equation, and Isoelectric Points. (2021, September 28) Retrieved Oct. 12, 2021, from https://chem.libretexts.org@go/page/36468). Thus the glutamate buffer would have buffering capacity around these values.

The “histidine buffer” refers to a buffer comprising histidine ions. The histidine buffer may comprise one or more of histidine, histidine hydrochloride, histidine acetate, histidine phosphate, histidine sulfate and the like. Histidine has the pKa values of 1.8, 9.2, as well as a side chain pKa of 6.0, and thus a histidine buffer would have buffering capacity around these values. In some embodiments, the histidine buffer is a histidine-histidine hydrochloride buffer. In some embodiments, the pH of the histidine buffer can be any pH value in the range of 5.5-6.5, such as 5.5, 5.6, 5.7, 5.8, 5.9, 6.0, 6.1, 6.2, 6.3, 6.4, or 6.5.

The term “aspartic acid buffer”, used interchangeably with “aspartate buffer”, refers to a buffer comprising aspartic acid optionally in equilibrium with its conjugate base. The buffer can be made from an aspartate salt, such as sodium aspartate, potassium aspartate, ammonium aspartate, calcium aspartate or magnesium aspartate. Aspartic acid has the pKa of 2.1, 9.8, as well as a side chain pKa of 3.9, detailed description of the pKa values of aspartic acid can be seen in, for example, Amino Acids, the Henderson-Hasselbalch Equation, and Isoelectric Points. (2021, September 28) Retrieved Oct. 12, 2021, from https://chem.libretexts.org/@go/page/36468). Thus an aspartic acid buffer would have buffering capacity around these values.

The “citrate buffer” is a buffer comprising citrate ions. The citrate buffer may comprise one or more of citric acid, monosodium citrate, disodium citrate, trisodium citrate, monopotassium citrate, dipotassium citrate, tripotassium citrate, sodium chloride, potassium chloride and the like. The pH of the citrate buffer can be any pH value in the range of 3.0-6.2.

The term “arginine buffer”, as used herein, refers to a buffer comprising arginine in equilibrium with its conjugate acid, such as HCl. Arginine has the pKa of 2.1, 9.0, as well as a side chain pKa of 12.5, detailed description of the pKa values of arginine can be seen in, for example, Amino Acids, the Henderson-Hasselbalch Equation, and Isoelectric Points. (2021, September 28) Retrieved Oct. 12, 2021, from https://chem.libretexts.org/@go/page/36468). Thus, the arginine buffer would have buffering capacity around these values.

The term “acetate buffer”, used interchangeably with “acetic acid buffer”, refers to a buffer comprising acetic acid in equilibrium with its respective conjugate base. The buffer can be made from an acetate salt, such as sodium acetate, potassium acetate, ammonium acetate, calcium acetate or magnesium acetate. The pH of the citrate buffer can be any pH value in the range of 3.6-5.8.

The term “glutamic acid & histidine buffer”, which can be used interchangeably with the term “glutamic acid and histidine buffer”, “glutamic acid/histidine buffer” or “glutamic/histidine buffer”, refers to a buffer system comprising glutamic acid buffer and histidine buffer, optionally with an acid or a base for adjusting final pH, such as HCl or NaOH. The glutamic acid portion of the glutamic acid & histidine buffer may comprise glutamic acid, glutamate ion and/or glutamate comprising glutamate salt, such as sodium, potassium, ammonium, calcium or magnesium salts of glutamate. This term includes both L and D forms of glutamic acid. In some embodiments, the glutamic acid & histidine buffer consists of glutamic acid and histidine, optionally with an acid or a base for adjusting final pH, such as HCl or NaOH. In some embodiments, the pH of the glutamic acid & histidine buffer can be any pH value in the range of 5.0-8.0.

The term “aspartic acid & histidine buffer”, which can be used interchangeably with the term “aspartic acid and histidine buffer” or “aspartic acid/histidine buffer”, refers to a buffer system comprising aspartic acid buffer and histidine buffer, optionally with an acid or a base for adjusting final pH, such as HCl or NaOH. The aspartic acid form of the aspartic acid & histidine buffer may comprise aspartic acid, aspartate ion and/or aspartate comprising aspartate salt, such as sodium, potassium, ammonium, calcium or magnesium salts of aspartate. This term includes both L and D forms of aspartic acid. In some embodiments, the aspartic acid & histidine buffer consists of aspartic acid and histidine, optionally with an acid or a base for adjusting final pH, such as HCl or NaOH. In some embodiments, the pH of the aspartic acid & histidine buffer can be any pH value in the range of 5.0-8.0.

The term “citrate & arginine buffer”, which can be used interchangeably with the term “citrate and arginine buffer” or “citrate/arginine buffer” refers to a buffer system comprising citric acid buffer and arginine buffer, optionally with an acid or a base for adjusting final pH, such as HCl or NaOH. In some embodiments, the citrate & arginine buffer consists of citric acid and arginine, optionally with an acid or a base for adjusting final pH, such as HCl or NaOH. In some embodiments, the pH of the citrate & arginine buffer can be any pH value in the range of 4.0-8.0.

In some embodiments, the pharmaceutical formulation of the present disclosure comprises a glutamic acid & histidine buffer or an aspartic acid & histidine buffer at a pH of about 5.0-8.0. In some embodiments, the pharmaceutical formulation of the present disclosure comprises a glutamic acid & histidine buffer consisting of glutamic acid and histidine, at a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0. In some embodiments, the pharmaceutical formulation of the present disclosure comprises a glutamic acid & histidine buffer consisting of glutamic acid and histidine, at a pH of about 5.0-6.0. In some embodiments, the pharmaceutical formulation of the present disclosure comprises a glutamic acid & histidine buffer consisting of glutamic acid and histidine, at a pH of about 5.0-5.5. In some embodiments, the pharmaceutical formulation of the present disclosure comprises a glutamic acid & histidine buffer consisting of glutamic acid and histidine, at a pH of about 5.0.

In some embodiments, the pharmaceutical formulation of the present disclosure comprises an aspartic acid & histidine buffer consisting of aspartic acid and histidine, at a pH of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0. In some embodiments, the pharmaceutical formulation of the present disclosure comprises an aspartic acid & histidine buffer consisting of aspartic acid and histidine, at a pH of about 5.0-6.0. In some embodiments, the pharmaceutical formulation of the present disclosure comprises an aspartic acid & histidine buffer consisting of aspartic acid and histidine, at a pH of about 5.0-5.5. In some embodiments, the pharmaceutical formulation of the present disclosure comprises an aspartic acid & histidine buffer consisting of aspartic acid and histidine, at a pH of about 5.0.

The concentration of the buffer, as used herein, refers to the concentration of buffer ions in the buffer. In some embodiments, the suitable concentration of buffers used in the pharmaceutical formulations of the present disclosure may be 1-100 mmol/L. In some embodiments, the concentration of the buffer is any concentration value within above range. For example, the concentration of the buffer is about 10-90 mmol/L, about 10-80 mmol/L, about 10-70 mmol/L, about 10-60 mmol/L, about 10-50 mmol/L, about 10-40 mmol/L, about 10-30 mmol/L, or about 20 mmol/L. In some embodiments, the concentration of the buffer is any concentration value within above range. For example, the concentration of the buffer is about 11-29 mmol/L, about 12-28 mmol/L, about 13-27 mmol/L, about 14-26 mmol/L, about 15-25 mmol/L, about 16-24 mmol/L, about 17-23 mmol/L, about 18-22 mmol/L, or about 19-21 mmol/L, depending on the specific buffer and the desired stability of the pharmaceutical formulation.

In some embodiments, the pharmaceutical formulation of the present disclosure comprises the buffer at a concentration of at least 2 mmol/L, at least 3 mmol/L, at least 4 mmol/L, at least 5 mmol/L, at least 6 mmol/L, at least 7 mmol/L, at least 8 mmol/L, at least 9 mmol/L, at least 11 mmol/L, at least 12 mmol/L, at least 13 mmol/L, at least 14 mmol/L, at least 15 mmol/L, at least 16 mmol/L, at least 17 mmol/L, at least 18 mmol/L, at least 19 mmol/L, at least 22 mmol/L, at least 22 mmol/L, at least 23 mmol/L, at least 24 mmol/L, at least 25 mmol/L, at least 26 mmol/L, at least 27 mmol/L, at least 28 mmol/L, at least 29 mmol/L, at least 31 mmol/L, at least 32 mmol/L, at least 33 mmol/L, at least 34 mmol/L, at least 35 mmol/L, at least 36 mmol/L, at least 37 mmol/L, at least 38 mmol/L, at least 39 mmol/L, at least 41 mmol/L, at least 42 mmol/L, at least 43 mmol/L, at least 44 mmol/L, at least 45 mmol/L, at least 46 mmol/L, at least 47 mmol/L, at least 48 mmol/L, at least 49 mmol/L, at least 51 mmol/L, at least 52 mmol/L, at least 53 mmol/L, at least 54 mmol/L, at least 55 mmol/L, at least 56 mmol/L, at least 57 mmol/L, at least 58 mmol/L, at least 59 mmol/L, at least 61 mmol/L, at least 62 mmol/L, at least 63 mmol/L, at least 64 mmol/L, at least 65 mmol/L, at least 66 mmol/L, at least 67 mmol/L, at least 68 mmol/L, at least 69 mmol/L, at least 71 mmol/L, at least 72 mmol/L, at least 73 mmol/L, at least 74 mmol/L, at least 75 mmol/L, at least 76 mmol/L, at least 77 mmol/L, at least 78 mmol/L, at least 79 mmol/L, at least 81 mmol/L, at least 82 mmol/L, at least 83 mmol/L, at least 84 mmol/L, at least 85 mmol/L, at least 86 mmol/L, at least 87 mmol/L, at least 88 mmol/L at least 89 mmol/L, at least 91 mmol/L, at least 92 mmol/L, at least 93 mmol/L, at least 94 mmol/L, at least 95 mmol/L, at least 96 mmol/L, at least 97 mmol/L, at least 98 mmol/L, or at least 99 mmol/L, depending on the specific buffer and the desired stability of the pharmaceutical formulation.

In some embodiments, the pharmaceutical formulation of the present disclosure comprises the buffer at a concentration of at most 2 mmol/L, at most 3 mmol/L, at most 4 mmol/L, at most 5 mmol/L, at most 6 mmol/L, at most 7 mmol/L, at most 8 mmol/L, at most 9 mmol/L, at most 11 mmol/L, at most 12 mmol/L, at most 13 mmol/L, at most 14 mmol/L, at most 15 mmol/L, at most 16 mmol/L, at most 17 mmol/L, at most 18 mmol/L, at most 19 mmol/L, at most 22 mmol/L, at most 22 mmol/L, at most 23 mmol/L, at most 24 mmol/L, at most 25 mmol/L, at most 26 mmol/L, at most 27 mmol/L, at most 28 mmol/L, at most 29 mmol/L, at most 31 mmol/L, at most 32 mmol/L, at most 33 mmol/L, at most 34 mmol/L, at most 35 mmol/L, at most 36 mmol/L, at most 37 mmol/L, at most 38 mmol/L, at most 39 mmol/L, at most 41 mmol/L, at most 42 mmol/L, at most 43 mmol/L, at most 44 mmol/L, at most 45 mmol/L, at most 46 mmol/L, at most 47 mmol/L, at most 48 mmol/L, at most 49 mmol/L, at most 51 mmol/L, at most 52 mmol/L, at most 53 mmol/L, at most 54 mmol/L, at most 55 mmol/L, at most 56 mmol/L, at most 57 mmol/L, at most 58 mmol/L, at most 59 mmol/L, at most 61 mmol/L, at most 62 mmol/L, at most 63 mmol/L, at most 64 mmol/L, at most 65 mmol/L, at most 66 mmol/L, at most 67 mmol/L, at most 68 mmol/L, at most 69 mmol/L, at most 71 mmol/L, at most 72 mmol/L, at most 73 mmol/L, at most 74 mmol/L, at most 75 mmol/L, at most 76 mmol/L, at most 77 mmol/L, at most 78 mmol/L, at most 79 mmol/L, at most 81 mmol/L, at most 82 mmol/L, at most 83 mmol/L, at most 84 mmol/L, at most 85 mmol/L, at most 86 mmol/L, at most 87 mmol/L, at most 88 mmol/L at most 89 mmol/L, at most 91 mmol/L, at most 92 mmol/L, at most 93 mmol/L, at most 94 mmol/L, at most 95 mmol/L, at most 96 mmol/L, at most 97 mmol/L, at most 98 mmol/L, or at most 99 mmol/L, depending on the specific buffer and the desired stability of the pharmaceutical formulation.

Other concentrations of the buffer are also within the contemplation of the present disclosure provided that the buffer has sufficient buffering capacity to maintain a selected pH of a formulation in certain scenarios, such as during manufacturing, packaging, sub-packaging, shipping, administration and/or storage.

In some embodiments, the pharmaceutical formulation of the present disclosure comprises a glutamic acid & histidine buffer. In some embodiments, the pharmaceutical formulation of the present disclosure comprises a glutamic acid & histidine buffer at a concentration of 1-100 mmol/L. In some embodiments, the pharmaceutical formulation of the present disclosure comprises a glutamic acid & histidine buffer at any concentration value within above range. For example, the pharmaceutical formulation of the present disclosure comprises a glutamic acid & histidine buffer at a concentration of about 10-90 mmol/L, about 10-80 mmol/L, about 10-70 mmol/L, about 10-60 mmol/L, about 10-50 mmol/L, about 10-40 mmol/L, about 10-30 mmol/L, or about 20 mmol/L. In some embodiments, the pharmaceutical formulation of the present disclosure comprises a glutamic acid & histidine buffer at a concentration of about 11-29 mmol/L, about 12-28 mmol/L, about 13-27 mmol/L, about 14-26 mmol/L, about 15-25 mmol/L, about 16-24 mmol/L, about 17-23 mmol/L, about 18-22 mmol/L, or about 19-21 mmol/L. In some embodiments, the pharmaceutical formulation of the present disclosure comprises a glutamic acid & histidine buffer consisting of glutamic acid and histidine, at any concentration value within the above ranges.

In some embodiments, the pharmaceutical formulation of the present disclosure comprises a glutamic acid & histidine buffer at a concentration of at least 2 mmol/L, at least 3 mmol/L, at least 4 mmol/L, at least 5 mmol/L, at least 6 mmol/L, at least 7 mmol/L, at least 8 mmol/L, at least 9 mmol/L, at least 11 mmol/L, at least 12 mmol/L, at least 13 mmol/L, at least 14 mmol/L, at least 15 mmol/L, at least 16 mmol/L, at least 17 mmol/L, at least 18 mmol/L, at least 19 mmol/L, at least 22 mmol/L, at least 22 mmol/L, at least 23 mmol/L, at least 24 mmol/L, at least 25 mmol/L, at least 26 mmol/L, at least 27 mmol/L, at least 28 mmol/L, at least 29 mmol/L, at least 31 mmol/L, at least 32 mmol/L, at least 33 mmol/L, at least 34 mmol/L, at least 35 mmol/L, at least 36 mmol/L, at least 37 mmol/L, at least 38 mmol/L, at least 39 mmol/L, at least 41 mmol/L, at least 42 mmol/L, at least 43 mmol/L, at least 44 mmol/L, at least 45 mmol/L, at least 46 mmol/L, at least 47 mmol/L, at least 48 mmol/L, at least 49 mmol/L, at least 51 mmol/L, at least 52 mmol/L, at least 53 mmol/L, at least 54 mmol/L, at least 55 mmol/L, at least 56 mmol/L, at least 57 mmol/L, at least 58 mmol/L, at least 59 mmol/L, at least 61 mmol/L, at least 62 mmol/L, at least 63 mmol/L, at least 64 mmol/L, at least 65 mmol/L, at least 66 mmol/L, at least 67 mmol/L, at least 68 mmol/L, at least 69 mmol/L, at least 71 mmol/L, at least 72 mmol/L, at least 73 mmol/L, at least 74 mmol/L, at least 75 mmol/L, at least 76 mmol/L, at least 77 mmol/L, at least 78 mmol/L, at least 79 mmol/L, at least 81 mmol/L, at least 82 mmol/L, at least 83 mmol/L, at least 84 mmol/L, at least 85 mmol/L, at least 86 mmol/L, at least 87 mmol/L, at least 88 mmol/L at least 89 mmol/L, at least 91 mmol/L, at least 92 mmol/L, at least 93 mmol/L, at least 94 mmol/L, at least 95 mmol/L, at least 96 mmol/L, at least 97 mmol/L, at least 98 mmol/L, or at least 99 mmol/L. In some embodiments, the pharmaceutical formulation of the present disclosure comprises a glutamic acid & histidine buffer consisting of glutamic acid and histidine, at any concentration value within the above ranges.

In some embodiments, the pharmaceutical formulation of the present disclosure comprises a glutamic acid & histidine buffer at a concentration of at most 2 mmol/L, at most 3 mmol/L, at most 4 mmol/L, at most 5 mmol/L, at most 6 mmol/L, at most 7 mmol/L, at most 8 mmol/L, at most 9 mmol/L, at most 11 mmol/L, at most 12 mmol/L, at most 13 mmol/L, at most 14 mmol/L, at most 15 mmol/L, at most 16 mmol/L, at most 17 mmol/L, at most 18 mmol/L, at most 19 mmol/L, at most 22 mmol/L, at most 22 mmol/L, at most 23 mmol/L, at most 24 mmol/L, at most 25 mmol/L, at most 26 mmol/L, at most 27 mmol/L, at most 28 mmol/L, at most 29 mmol/L, at most 31 mmol/L, at most 32 mmol/L, at most 33 mmol/L, at most 34 mmol/L, at most 35 mmol/L, at most 36 mmol/L, at most 37 mmol/L, at most 38 mmol/L, at most 39 mmol/L, at most 41 mmol/L, at most 42 mmol/L, at most 43 mmol/L, at most 44 mmol/L, at most 45 mmol/L, at most 46 mmol/L, at most 47 mmol/L, at most 48 mmol/L, at most 49 mmol/L, at most 51 mmol/L, at most 52 mmol/L, at most 53 mmol/L, at most 54 mmol/L, at most 55 mmol/L, at most 56 mmol/L, at most 57 mmol/L, at most 58 mmol/L, at most 59 mmol/L, at most 61 mmol/L, at most 62 mmol/L, at most 63 mmol/L, at most 64 mmol/L, at most 65 mmol/L, at most 66 mmol/L, at most 67 mmol/L, at most 68 mmol/L, at most 69 mmol/L, at most 71 mmol/L, at most 72 mmol/L, at most 73 mmol/L, at most 74 mmol/L, at most 75 mmol/L, at most 76 mmol/L, at most 77 mmol/L, at most 78 mmol/L, at most 79 mmol/L, at most 81 mmol/L, at most 82 mmol/L, at most 83 mmol/L, at most 84 mmol/L, at most 85 mmol/L, at most 86 mmol/L, at most 87 mmol/L, at most 88 mmol/L at most 89 mmol/L, at most 91 mmol/L, at most 92 mmol/L, at most 93 mmol/L, at most 94 mmol/L, at most 95 mmol/L, at most 96 mmol/L, at most 97 mmol/L, at most 98 mmol/L, or at most 99 mmol/L. In some embodiments, the pharmaceutical formulation of the present disclosure comprises a glutamic acid & histidine buffer consisting of glutamic acid and histidine, at any concentration value within the above ranges.

Since the aspartic acid differs from glutamic acid only by one methylene group, it can be therefore expected that similar technical effect (e.g., maintain the correct pH of a finished pharmaceutical formulation) would be achieved when the glutamic acid in the glutamic acid & histidine buffer mentioned above is replaced with the aspartic acid.

### Stabilizers

The pharmaceutical formulation of the present disclosure may comprise one or more stabilizers. As used herein, the term “stabilizer” refers to an agent that can facilitate maintenance of an API's structure and/or to minimize electrostatic, protein-protein interactions, and/or refers to an agent that confers desired osmolarity (e.g., isotonicity, hypotonicity or hypertonicity) to a pharmaceutical formulation, such that the finished pharmaceutical formulation would be suitable for administration. In other words, the stabilizer used in the pharmaceutical formulations of the present disclosure can also serve the function of an isotonic agent that can impart a suitable osmotic tension to a drug to avoid the net flow of water across the cell membrane that contacts the drug. In some embodiments, the formulation of the present disclosure has substantially the same osmotic pressure as human blood.

Exemplary stabilizers include, but not limited to, polyols (e.g., sorbitol, mannitol), sugars (e.g., glucose, sucrose, trehalose, lactose, dextrose), and/or salts (e.g., sodium chloride, sodium sulfate, ammonium acetate, potassium chloride, calcium phosphate).

In some embodiments, the stabilizer used in the pharmaceutical formulations of the present disclosure is selected from sugars. In some embodiments, the stabilizer used in the pharmaceutical formulations of the present disclosure is selected from the group consisting of sucrose, trehalose, or combinations thereof. In some embodiments, the stabilizer used in the pharmaceutical formulations of the present disclosure is selected from polyols. In some embodiments, the stabilizer used in the pharmaceutical formulations of the present disclosure is selected from the group consisting of sorbitol, mannitol, or combinations thereof.

In some embodiments, the type and concentration of the stabilizers used in the pharmaceutical formulation of the present disclosure can be determined based on the desired osmolarity of the final formulation. For example, about 5% sorbitol can achieve isotonicity while about 9% sucrose would be required to achieve isotonicity. In certain embodiments, the pharmaceutical formulation of the present disclosure comprises stabilizer at a concentration of about 0.5%-50% (w/v). In some embodiments, the concentration of the stabilizer is any value within above range. For example, the pharmaceutical formulation of the present disclosure comprises a stabilizer at a concentration of about 1% to about 40% (w/v), about 1.5% to about 39.5% (w/v), about 2% to about 39% (w/v), about 2.5% to about 38.5% (w/v), about 3% to about 38% (w/v), about 3% to about 36% (w/v), about 3% to about 34% (w/v), about 3% to about 32% (w/v), about 3% to about 30% (w/v), about 3% to about 28% (w/v), about 3% to about 26% (w/v), about 3% to about 24% (w/v), about 3% to about 22% (w/v), about 3% to about 20% (w/v), about 3.2% to about 18% (w/v), about 3.4% to about 16% (w/v), about 2% to about 30% (w/v), about 3% to about 20% (w/v), about 3.2% to about 18% (w/v), about 3.4% to about 16% (w/v), about 3.6% to about 14% (w/v), about 4% to about 12% (w/v), about 6% to about 10% (w/v), or about 8% (w/v).

In some embodiments, the pharmaceutical formulation of the present disclosure comprises a stabilizer at a concentration of at least 0.5% (w/v), at least 1% (w/v), at least 1.5% (w/v), at least 2% (w/v), at least 2.5% (w/v), at least 3% (w/v), at least 3.5% (w/v), at least 4% (w/v), at least 4.5% (w/v), at least 5% (w/v), at least 5.5% (w/v), at least 6% (w/v), at least 6.5% (w/v), at least 7% (w/v), at least 7.5% (w/v), at least 8% (w/v), at least 8.1% (w/v), at least 8.2% (w/v), at least 8.3% (w/v), at least 8.4% (w/v), at least 8.5% (w/v), at least 8.6% (w/v), at least 8.7% (w/v), at least 8.8% (w/v), at least 8.9% (w/v), at least 9% (w/v), at least 9.5% (w/v), at least 10% (w/v), at least 10.5% (w/v), at least 11% (w/v), at least 11.5% (w/v), at least 12% (w/v), at least 12.5% (w/v), at least 13% (w/v), at least 13.5% (w/v), at least 14% (w/v), at least 14.5% (w/v), at least 15% (w/v), at least 15.5% (w/v), at least 16% (w/v), at least 16.5% (w/v), at least 17% (w/v), at least 17.5% (w/v), at least 18% (w/v), at least 18.5% (w/v), at least 19% (w/v), at least 19.5% (w/v), at least 20% (w/v), at least 20.5% (w/v), at least 21% (w/v), at least 21.5% (w/v), at least 22% (w/v), at least 22.5% (w/v), at least 23% (w/v), at least 23.5% (w/v), at least 24% (w/v), at least 24.5% (w/v), at least 25% (w/v), at least 25.5% (w/v), at least 26% (w/v), at least 26.5% (w/v), at least 27% (w/v), at least 27.5% (w/v), at least 28% (w/v), at least 28.5% (w/v), at least 29% (w/v), at least 29.5% (w/v), at least 30% (w/v), at least 30.5% (w/v), at least 31% (w/v), at least 31.5% (w/v), at least 32% (w/v), at least 32.5% (w/v), at least 33% (w/v), at least 33.5% (w/v), at least 34% (w/v), at least 34.5% (w/v), at least 35% (w/v), at least 35.5% (w/v), at least 36% (w/v), at least 36.5% (w/v), at least 37% (w/v), at least 37.5% (w/v), at least 38% (w/v), at least 38.5% (w/v), at least 39% (w/v), at least 39.5% (w/v), at least 40% (w/v), at least 40.5% (w/v), at least 41% (w/v), at least 41.5% (w/v), at least 42% (w/v), at least 42.5% (w/v), at least 43% (w/v), at least 43.5% (w/v), at least 44% (w/v), at least 44.5% (w/v), at least 45% (w/v), at least 45.5% (w/v), at least 46% (w/v), at least 46.5% (w/v), at least 47% (w/v), at least 47.5% (w/v), at least 48% (w/v), at least 48.5% (w/v), at least 49% (w/v), or at least 49.5% (w/v), depending on the specific stabilizer and the desired stability of pharmaceutical formulation.

In some embodiments, the pharmaceutical formulation of the present disclosure comprises a stabilizer at a concentration of at most 1% (w/v), at most 1.5% (w/v), at most 2% (w/v), at most 2.5% (w/v), at most 3% (w/v), at most 3.5% (w/v), at most 4% (w/v), at most 4.5% (w/v), at most 5% (w/v), at most 5.5% (w/v), at most 6% (w/v), at most 6.5% (w/v), at most 7% (w/v), at most 7.5% (w/v), at most 8% (w/v), at most 8.1% (w/v), at most 8.2% (w/v), at most 8.3% (w/v), at most 8.4% (w/v), at most 8.5% (w/v), at most 8.6% (w/v), at most 8.7% (w/v), at most 8.8% (w/v), at most 8.9% (w/v), at most 9% (w/v), at most 9.5% (w/v), at most 10% (w/v), at most 10.5% (w/v), at most 11% (w/v), at most 11.5% (w/v), at most 12% (w/v), at most 12.5% (w/v), at most 13% (w/v), at most 13.5% (w/v), at most 14% (w/v), at most 14.5% (w/v), at most 15% (w/v), at most 15.5% (w/v), at most 16% (w/v), at most 16.5% (w/v), at most 17% (w/v), at most 17.5% (w/v), at most 18% (w/v), at most 18.5% (w/v), at most 19% (w/v), at most 19.5% (w/v), at most 20% (w/v), at most 20.5% (w/v), at most 21% (w/v), at most 21.5% (w/v), at most 22% (w/v), at most 22.5% (w/v), at most 23% (w/v), at most 23.5% (w/v), at most 24% (w/v), at most 24.5% (w/v), at most 25% (w/v), at most 25.5% (w/v), at most 26% (w/v), at most 26.5% (w/v), at most 27% (w/v), at most 27.5% (w/v), at most 28% (w/v), at most 28.5% (w/v), at most 29% (w/v), at most 29.5% (w/v), at most 30% (w/v), at most 30.5% (w/v), at most 31% (w/v), at most 31.5% (w/v), at most 32% (w/v), at most 32.5% (w/v), at most 33% (w/v), at most 33.5% (w/v), at most 34% (w/v), at most 34.5% (w/v), at most 35% (w/v), at most 35.5% (w/v), at most 36% (w/v), at most 36.5% (w/v), at most 37% (w/v), at most 37.5% (w/v), at most 38% (w/v), at most 38.5% (w/v), at most 39% (w/v), at most 39.5% (w/v), at most 40% (w/v), at most 40.5% (w/v), at most 41% (w/v), at most 41.5% (w/v), at most 42% (w/v), at most 42.5% (w/v), at most 43% (w/v), at most 43.5% (w/v), at most 44% (w/v), at most 44.5% (w/v), at most 45% (w/v), at most 45.5% (w/v), at most 46% (w/v), at most 46.5% (w/v), at most 47% (w/v), at most 47.5% (w/v), at most 48% (w/v), at most 48.5% (w/v), at most 49% (w/v), at most 49.5% (w/v), or at most 50% (w/v), depending on the specific stabilizer and the desired stability of pharmaceutical formulation.

In some embodiments, the pharmaceutical formulation of the present disclosure comprises sucrose. In some embodiments, the pharmaceutical formulation of the present disclosure comprises sucrose at a concentration of about 4%-12% (w/v), about 5%-11% (w/v), about 6%-10% (w/v), about 7%-9% (w/v) or about 8% (w/v). In some embodiments, the pharmaceutical formulation of the present disclosure comprises sucrose at a concentration of about 8% (w/v).

In some embodiments, the pharmaceutical formulation of the present disclosure comprises sorbitol. In some embodiments, the pharmaceutical formulation of the present disclosure comprises sorbitol at a concentration of about 0.5%-10% (w/v), about 1%-9.5% (w/v), about 1.5%-9% (w/v), about 2%-8.5% (w/v), about 2.5%-8% (w/v), about 3%-7.5% (w/v), about 3.5%-7% (w/v), about 4%-6.5% (w/v), about 4.5%-6% (w/v), about 5%-5.5% (w/v), or about 4.5% (w/v). In some embodiments, the pharmaceutical formulation of the present disclosure comprises sorbitol at a concentration of about 4.5% (w/v).

In some embodiments, the pharmaceutical formulation of the present disclosure comprises trehalose. In some embodiments, the pharmaceutical formulations of the present disclosure comprises trehalose at a concentration of about 4%-14% (w/v), about 5%-13% (w/v), about 6%-12% (w/v), about 7%-11% (w/v), about 7.5%-10.5% (w/v), about 8%-10% (w/v), about 8.2%-9.8% (w/v), about 8.4%-9.6% (w/v), about 8.6%-9.4% (w/v), about 8.8%-9.2% (w/v), about 9.0% (w/v), or about 8.8% (w/v). In some embodiments, the pharmaceutical formulation of the present disclosure comprises trehalose at a concentration of about 8.8% (w/v).

In some embodiments, the pharmaceutical formulation of the present disclosure has an osmolarity in a range of about 200-400 mOsmol·kg, about 250-350 mOsmol·kg, about 280-320 mOsmol·kg, about 285 mOsmol·kg, about 290 mOsmol·kg, or about 300 mOsmol·kg. In some embodiments, the pharmaceutical formulation of the present disclosure has an osmolarity of 300±10 mOsmol·kg.

### Surfactants

The pharmaceutical formulation of the present disclosure may further comprise one or more surfactants to, for example, adjust osmolarity, prevent, control, or minimize aggregation (e.g., interface induced aggregation), particle formation and/or surface adsorption (e.g., surface-induced degradation) during the process of liquid formulations, lyophilization, reconstitution of lyophilized formulations, and/or transportation of the pharmaceutical formulation. Specifically, a surface layer of a surfactant can prevent protein molecules from adsorbing at the interface at sufficient concentrations (e.g., about the surfactant's micellar concentration), and as such surface-induced degradation of the APIs can be minimized. As used herein, the term “surfactant” refers to a substance (e.g., an organic material having an amphiphilic structure that is both hydrophilic and hydrophobic) that functions to reduce the surface tension of a liquid where the substance is dissolved. Surfactants can be classified into ionic (e.g., anionic, cationic) and non-ionic surfactants depending on the charge of the surface active moiety. Surfactants are well-known in the art and description of which can be found in, for example, Randolph T. W. and Jones L. S., Surfactant-protein interactions. Pharm Biotechnol. 13:159-75 (2002).

Exemplary ionic surfactants include, but not limited to, anionic, cationic and zwitterionic surfactants. Exemplary anionic surfactants include, but not limited to, sulfonate-based surfactants or carboxylate-based surfactants, such as fatty acid salts, soaps, ammonium lauryl sulfate, sodium dodecyl sulfate (SDS) and other alkyl sulfate salts. Exemplary cationic surfactants include, but not limited to, quaternary ammonium-based surfactants, such as acetyl pyridinium chloride, benzalkonium chloride, acetyl trimethylammonium bromide (CTAB), and polyethoxylated tallow amine (POEA). Exemplary zwitterionic surfactants include, but not limited to, dodecyl dimethylamine oxide, cocamidopropyl betaine, dodecyl betaine and coco ampho glycinate.

Exemplary non-ionic surfactants include, but not limited to, alkyl poly (ethylene oxide), alkyl polyglucosides (e.g., octyl glucoside and decyl maltoside), fatty alcohols (e.g., acetyl alcohol and oleyl alcohol), cocamide DEA, cocamide MWA, cocamide TEA, the poloxamers (e.g., poloxamer 188, poloxamer 407), Triton, polyethylene glycol, polypropylene glycol and copolymers of ethylene glycol and propylene glycol (e.g., Pluronics, PF68 etc.), and the polysorbates (e.g., polysorbate 20, polysorbate 28, polysorbate 40, polysorbate 60, polysorbate 65, polysorbate 80, polysorbate 81, and polysorbate 85).

In some embodiments, the pharmaceutical formulation of the present disclosure comprises a surfactant at a concentration of about 0.001-0.1% w/v. In some embodiments, the concentration of surfactant is any value within above range. For example, the pharmaceutical formulation of the present disclosure comprises a surfactant at a concentration of about 0.002% to about 0.08% (w/v), about 0.004% to about 0.06% (w/v), about 0.006% to about 0.05% (w/v), about 0.008% to about 0.05% (w/v), about 0.01% to about 0.05% (w/v), about 0.02% to about 0.04% (w/v), about 0.02% to about 0.03% (w/v), or about 0.02% (w/v).

In some embodiments, the pharmaceutical formulation of the present disclosure comprises a surfactant at a concentration of at least 0.002% (w/v), at least 0.003% (w/v), at least 0.004% (w/v), at least 0.005% (w/v), at least 0.006% (w/v), at least 0.007% (w/v), at least 0.008% (w/v), at least 0.009% (w/v), at least 0.01% (w/v), at least 0.012% (w/v), at least 0.014% (w/v), at least 0.016% (w/v), at least 0.018% (w/v), at least 0.02% (w/v), at least 0.022% (w/v), at least 0.024% (w/v), at least 0.026% (w/v), at least 0.028% (w/v), at least 0.03% (w/v), at least 0.032% (w/v), at least 0.034% (w/v), at least 0.036% (w/v), at least 0.038% (w/v), at least 0.04% (w/v), at least 0.042% (w/v), at least 0.044% (w/v), at least 0.046% (w/v), at least 0.048% (w/v), at least 0.05% (w/v), at least 0.052% (w/v), at least 0.054% (w/v), at least 0.056% (w/v), at least 0.058% (w/v), at least 0.06% (w/v), at least 0.062% (w/v), at least 0.064% (w/v), at least 0.066% (w/v), at least 0.068% (w/v), at least 0.07% (w/v), at least 0.072% (w/v), at least 0.074% (w/v), at least 0.076% (w/v), at least 0.078% (w/v), at least 0.08% (w/v), at least 0.082% (w/v), at least 0.084% (w/v), at least 0.086% (w/v), at least 0.088% (w/v), at least 0.09% (w/v), at least 0.092% (w/v), at least 0.094% (w/v), at least 0.096% (w/v), or at least 0.098% (w/v), depending on the specific surfactant and the desired stability of pharmaceutical formulation.

In some embodiments, the pharmaceutical formulation of the present disclosure comprises a surfactant at a concentration of at most 0.002% (w/v), at most 0.003% (w/v), at most 0.004% (w/v), at most 0.005% (w/v), at most 0.006% (w/v), at most 0.007% (w/v), at most 0.008% (w/v), at most 0.009% (w/v), at most 0.01% (w/v), at most 0.012% (w/v), at most 0.014% (w/v), at most 0.016% (w/v), at most 0.018% (w/v), at most 0.02% (w/v), at most 0.022% (w/v), at most 0.024% (w/v), at most 0.026% (w/v), at most 0.028% (w/v), at most 0.03% (w/v), at most 0.032% (w/v), at most 0.034% (w/v), at most 0.036% (w/v), at most 0.038% (w/v), at most 0.04% (w/v), at most 0.042% (w/v), at most 0.044% (w/v), at most 0.046% (w/v), at most 0.048% (w/v), at most 0.05% (w/v), at most 0.052% (w/v), at most 0.054% (w/v), at most 0.056% (w/v), at most 0.058% (w/v), at most 0.06% (w/v), at most 0.062% (w/v), at most 0.064% (w/v), at most 0.066% (w/v), at most 0.068% (w/v), at most 0.07% (w/v), at most 0.072% (w/v), at most 0.074% (w/v), at most 0.076% (w/v), at most 0.078% (w/v), at most 0.08% (w/v), at most 0.082% (w/v), at most 0.084% (w/v), at most 0.086% (w/v), at most 0.088% (w/v), at most 0.09% (w/v), at most 0.092% (w/v), at most 0.094% (w/v), at most 0.096% (w/v), at most 0.098% (w/v), or at most 0.1% (w/v), depending on the specific surfactant and the desired stability of pharmaceutical formulation.

In some embodiments, the pharmaceutical formulation of the present disclosure comprises polysorbate 20. In some embodiments, the pharmaceutical formulation of the present disclosure comprises polysorbate 80. In some embodiments, the pharmaceutical formulation of the present disclosure comprises polysorbate 80 at a concentration of about 0.01-0.05% (w/v), about 0.012-0.048% (w/v), about 0.014-0.046% (w/v), about 0.016-0.044% (w/v), about 0.018-0.042% (w/v), about 0.02-0.04% (w/v), about 0.022-0.038% (w/v), about 0.024-0.036% (w/v), about 0.026-0.034% (w/v), about 0.028-0.032% (w/v), or about 0.03% (w/v). In some embodiments, the pharmaceutical formulation of the present disclosure comprises polysorbate 80 at a concentration of about 0.02% (w/v).

### Other Materials

The pharmaceutical formulation of the present disclosure may further comprise one or more other excipients as those described in Remington's Pharmaceutical Sciences 16th edition, Osol, A. Ed. (1980), such as diluents, provided that the one or more other excipients do not adversely affect the desired features of the pharmaceutical formulations of the present disclosure.

The term “diluent” is pharmaceutically acceptable and can be used to dilute the pharmaceutical formulations of the present disclosure. Typical diluents include water, physiological saline, antibacterial agents for injection, pH buffer, sterile salt solution, Ringer solution, or glucose solution. In certain embodiments, the pharmaceutical formulation of the present disclosure further comprises a diluent comprising 0.9% normal saline or 5% dextrose.

### APIs

The skilled person in the art understand that the pharmaceutical formulations described herein can be equally applicable to many types of APIs, including those exemplified (e.g., antibodies or antigen-binding fragments thereof), as well as other APIs known in the art.

In some embodiments, the API of the pharmaceutical formulations of the present disclosure is a monoclonal antibody or antigen-binding fragment thereof.

As used herein, the term “monoclonal antibody” refers to a population of antibodies that comprises a homogeneous or substantially homogeneous single antibody. Monoclonal antibodies can be obtained from a single hybridoma cell clone (Milstein, C (1999). “The hybridoma revolution: an offshoot of basic research”. BioEssays. 21 (11): 966-73). A complete monoclonal antibody comprises two heavy chains and two light chains. Each heavy chain consists of a heavy chain variable region (V_{H}) and a first, second, and third constant regions (C_{H1}, C_{H2}, C_{H3}). Each light chain consists of a light chain variable region (V_{L}) and a light chain constant region (C_{L}). Each of the V_{H} and V_{L} in heavy and light chains contains three complementarity determining regions (CDRs). The three CDRs are separated by contiguous portions known as framework regions (FRs), which are more highly conserved than the CDRs and form a scaffold to support the hypervariable loops. The six CDRs of one heavy chain and one light chain together constitute the antigen binding portion of the antibody and determine the specificity of the antibody. The monoclonal antibodies described herein also comprise fragments or derivatives of a complete monoclonal antibody that have an antigen binding function. The fragments or derivatives have the same antigen binding specificity as the complete monoclonal antibody, but the affinity of the fragments or derivatives for binding to their specific antigen may be the same as or different from that of the complete monoclonal antibody.

In some embodiments, the monoclonal antibody described herein comprises antigen-binding fragments. An antigen-binding fragment refers to one or more antibody fragments that retain the ability to specifically bind to an antigen. Examples of antigen-binding fragments include, without limitation, (i) a Fab fragment, which refers to a monovalent fragment composed of V_{L}, V_{H}, C_{L}, and C_{H1} domains; (ii) a Fab′ fragment, which refers to a Fab fragment that comprises a portion of the hinge region; (iii) a F(ab′)₂ fragment, which refers to a bivalent fragment comprising two Fab fragments linked by a disulfide bond in the hinge region; (iv) a Fd fragment consisting of V_{H} and C_{H1} domains; (v) a Fv fragment consisting of the V_{L} and V_{H} domains of a single arm of the antibody; (vi) a dAb fragment (Ward et al., Nature 341:544-546 (1989); PCT Publication WO 90/05144) comprising a single variable domain; (vii) an isolated CDR; (viii) a single-chain Fv fragment, which refers to a monovalent fragment formed from the V_{L} and V_{H} domains that are linked directly or linked via a peptide chain (Huston J S et al., Proc Natl Acad Sci USA, 85:5879(1988)).

In some embodiments, the monoclonal antibody described herein comprise a chimeric monoclonal antibody in which a portion of the heavy chain and/or light chain is identical or homologous with a corresponding sequence of antibodies derived from a particular species or belonging to a particular antibody class or subclass, and the rest chain is identical or homologous with a corresponding sequence of antibodies and the fragments thereof deriving from the other species or belonging to the other antibody class or subclass, as long as they exhibit the desired functional activity.

In some embodiments, the monoclonal antibodies described herein include human murine chimeric monoclonal antibodies having murine heavy chain and light chain variable regions, and human heavy chain and light chain constant regions.

In some embodiments, the monoclonal antibodies described herein include humanized monoclonal antibodies. A humanized form of a non-human (e.g., murine) antibody is a chimeric immunoglobulin, immunoglobulin chain or fragment thereof (e.g., Fv, Fab, Fab′, F(ab′)₂ or other antigen-binding sequences of the antibody) containing minimal sequences obtained from non-human immunoglobulin. In some examples, the humanized antibody may be a CDR-grafted antibody in which the amino acid sequence of a human CDR is introduced into the amino acid sequences of non-human V_{H} and V_{L} to replace the amino acid sequence of the corresponding non-human CDR. In other examples, most of the amino acid sequences of humanized antibodies may be derived from human immunoglobulins (receptor antibodies) where the amino acid residues of the CDRs of the receptor are replaced by the amino acid residues of the CDRs of non-human (e.g., mouse, rat, rabbit) antibody having the desired specificity, affinity and ability. In general, humanized antibodies comprise essentially at least one, and generally two variable domains, wherein all or substantially all of the CDR regions correspond to the sequence of a non-human immunoglobulin, and all or substantially all of the framework (FR) region is the sequence of human immunoglobulin. In some examples, the framework region residues of the variable regions of human immunoglobulins are replaced by the corresponding non-human residues. Moreover, a humanized antibody can comprise residues that are not found in either the receptor antibody or the imported CDR or framework region sequences.

In certain embodiments, the API of the pharmaceutical formulations of the present disclosure is a monoclonal anti-OX40 antibody or antigen-binding fragment thereof. The monoclonal anti-OX40 antibody described herein refers to a monoclonal antibody that specifically binds to the OX40 receptor protein. In certain embodiments, the monoclonal anti-OX40 antibody or antigen-binding fragment thereof of the pharmaceutical formulations of the present disclosure is an antagonist antibody that has a blocking activity for OX40-mediated signal transduction.

The OX40 receptor protein, also known as CD134 or tumor necrosis factor receptor superfamily, member 4 (TNFRSF4), is a member of the TNFR-superfamily of receptors. OX40 is a secondary co-stimulatory immune checkpoint molecule, which is not constitutively expressed on resting naive T cells, and can be expressed after T cell activation. Binding of OX40 ligand to OX40 receptors on T cells would prevent the T cells from dying and subsequently increase cytokine production. The monoclonal anti-OX40 antibody or antigen-binding fragment thereof of the pharmaceutical formulations of the present disclosure can block the binding of OX40 ligand to OX40 to prevent the trimerization of OX40, and hence to inhibit T cell activation and related inflammatory responses induced by OX40 activation.

In some embodiments, the monoclonal anti-OX40 antibodies described herein comprise a heavy chain and a light chain, wherein the heavy chain comprises a heavy chain variable region V_{H} that comprises heavy chain CDR1 (HCDR1) comprising the amino acid sequence as set forth in SEQ ID NO: 1, HCDR2 comprising the amino acid sequence as set forth in SEQ ID NO: 2 and HCDR3 comprising the amino acid sequence as set forth in SEQ ID NO: 3, wherein the light chain comprises a light chain variable region V_{L} that comprises light chain CDR1 (LCDR1) comprising the amino acid sequence as set forth in SEQ ID NO: 4, LCDR2 comprising the amino acid sequence as set forth in SEQ ID NO: 5 and LCDR3 comprising the amino acid sequence as set forth in SEQ ID NO: 6.

In some embodiments, the monoclonal anti-OX40 antibody described herein comprises a heavy chain variable region V_{H} comprising the amino acid sequence selected from the group consisting of SEQ ID NO: 7, SEQ ID NO: 9, and SEQ ID NO: 10. In some embodiments, the monoclonal anti-OX40 antibody described herein comprises a light chain variable region V_{L} comprising the amino acid sequence selected from the group consisting of SEQ ID NO: 8, SEQ ID NO: 11 and SEQ ID NO: 12. In some embodiments, the monoclonal anti-OX40 antibody described herein comprises a heavy chain variable region V_{H} comprising the amino acid sequence of SEQ ID NO: 7. In some embodiments, the monoclonal anti-OX40 antibody described herein comprises a light chain variable region V_{L} comprising the amino acid sequence of SEQ ID NO: 8. In some embodiments, the monoclonal anti-OX40 antibody described herein comprises a heavy chain variable region V_{H} comprising the amino acid sequence of SEQ ID NO: 7 and a light chain variable region V_{L} comprising the amino acid sequence of SEQ ID NO: 8. In some embodiments, the monoclonal anti-OX40 antibody described herein comprises a heavy chain variable region V_{H} comprising the amino acid sequence of SEQ ID NO: 9 and a light chain variable region V_{L} comprising the amino acid sequence of SEQ ID NO: 11. In some embodiments, the monoclonal anti-OX40 antibody described herein comprises a heavy chain variable region V_{H} comprising the amino acid sequence of SEQ ID NO: 10 and a light chain variable region V_{L} comprising the amino acid sequence of SEQ ID NO: 8. In some embodiments, the monoclonal anti-OX40 antibody described herein comprises a heavy chain variable region V_{H} comprising the amino acid sequence of SEQ ID NO: 7 and a light chain variable region V_{L} comprising the amino acid sequence of SEQ ID NO: 11. In some embodiments, the monoclonal anti-OX40 antibody described herein comprises a heavy chain variable region V_{H} comprising the amino acid sequence of SEQ ID NO: 10 and a light chain variable region V_{L} comprising the amino acid sequence of SEQ ID NO: 12.

In some embodiments, the monoclonal anti-OX40 antibody described herein further comprises an immunoglobulin constant region. In some embodiments, the immunoglobulin constant region comprises a heavy chain constant region and/or a light chain constant region. The heavy chain constant region comprises C_{H1}, C_{H1}-C_{H2}, or C_{H1}-C_{H3} region, and the light chain constant region comprises a C_{L} region. In some embodiments, the monoclonal anti-OX40 antibodies described herein further comprises a Fc region variant, which is human IgG1 N297A. The term “IgG1 N297A” refers to an Fc region variant of IgG1 that has the substitution of asparagine with alanine at position 297 relative to the parent polypeptide (i.e., the wild-type Fc region of IgG1), where the number is in accordance with EU index.

In some embodiments, the monoclonal anti-OX40 antibody described herein is a monoclonal antibody that comprises a heavy chain comprising the amino acid sequence of SEQ ID NO: 13 and a light chain comprising the amino acid sequence of SEQ ID NO: 14. The nucleic acid sequences encoding the heavy chain and light chain of the monoclonal anti-OX40 antibody described herein comprise SEQ ID NO: 15 and SEQ ID NO: 16 respectively.

The APIs as described above may comprise additional post translational modifications, such as glycosylation, oxidation and deamidation. For example, the monoclonal anti-OX40 antibody described herein may comprise glycosylation site, oxidation site and/or deamidation site in its V_{H} and/or V_{L}. In particular, for the monoclonal anti-XO40 antibody having a V_{H} comprising SEQ ID NO: 7 and a V_{L} comprising SEQ ID NO: 8, the potential oxidation sites are bolded in the amino acid sequences as set forth in SEQ ID NO: 7 and SEQ ID NO: 8 in Table 1 below. The potential deamidation sites are in italics and bolded in the amino acid sequences as set forth in SEQ ID NO: 7 and SEQ ID NO: 8 in Table 1 below. The potential isomerization sites are underlined and bolded in the amino acid sequences as set forth in SEQ ID NO: 7 and SEQ ID NO: 8 in Table 1 below.

Exemplary amino acid sequences in some embodiments are listed in Table 1 below:

TABLE 1
Exemplary Amino Acid Sequences and
Nucleic Acid Sequences

| SEQ | |
|---|---|
| ID | |
| NO. | Sequence |
| 1 | SYWVD |
| 2 | NIYPSDSETHYNQKFKD |
| 3 | SYGYYGTWFAY |
| 4 | RASESVDSSGNSEMH |
| 5 | RASNLES |
| 6 | QQSNEDPWT |
| 7 | QVQLVQSGAEVKKPGASVKVSCKASGYTFTSYWVDWVRQAPGQGL |
| | EWMGNIYPSDSETHYNQKFKDRVTMTRDTSTSTVYMELSSLRSED |
| | TAVYYCARSYGYYGTWFAYWGQGTLVTVSS |
| 8 | DIVMTQSPDSLAVSLGERATINCRASESVDSSGNSEMHWYQQKPG |
| | QPPKLLIYRASNLESGVPDRESGSGSGTDFTLTISSLQAEDVAVY |
| | YCQQSNEDPWTFGGGTKLEIK |
| 9 | QVQLVQSGAEVKKPGASVKVSCKASGYTFTSYWVDWVRQAPGQGL |
| | EWIGNIYPSDSETHYNQKFKDRVTMTVDTSTSTVYMELSSLRSED |
| | TAVYYCARSYGYYGTWFAYWGQGTLVTVSS |
| 10 | QVQLVQSGAEVKKPGASVKVSCKASGYTFTSYWVDWVRQAPGQGL |
| | EWIGNIYPSDSETHYNQKFKDRVTMTVDTSTSTAYMELSSLRSED |
| | SAVYYCARSYGYYGTWFAYWGQGTLVTVSS |
| 11 | DIVMTQSPDSLAVSLGERATINCRASESVDSSGNSFMHWYQQKPG |
| | QPPKLLIYRASNLESGIPDRESGSGSGTDFTLTISSVQAEDVAVY |
| | YCQQSNEDPWTFGGGTKLEIK |
| 12 | DIVMTQSPDSLAVSLGERATINCRASESVDSSGNSFMHWYQQKPG |
| | QPPKLLIYRASNLESGIPDRFSGSGSGTDFTLTISSLQAEDVAVY |
| | YCQQSNEDPWTFGGGTKLEIK |
| 13 | QVQLVQSGAEVKKPGASVKVSCKASGYTFTSYWVDWVRQAPGQGL |
| | EWMGNIYPSDSETHYNQKFKDRVTMTRDTSTSTVYMELSSLRSED |
| | TAVYYCARSYGYYGTWFAYWGQGTLVTVSSASTKGPSVFPLAPSS |
| | KSTSGGTAALGCLVKDYFPEPVTVSWNSGALTSGVHTFPAVLQSS |
| | GLYSLSSVVTVPSSSLGTQTYICNVNHKPSNTKVDKRVEPKSCDK |
| | THTCPPCPAPELLGGPSVFLFPPKPKDTLMISRTPEVTCVVVDVS |
| | HEDPEVKFNWYVDGVEVHNAKTKPREEQYASTYRVVSVLTVLHQD |
| | WLNGKEYKCKVSNKALPAPIEKTISKAKGOPREPQVYTLPPSREE |
| | MTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| 14 | DIVMTQSPDSLAVSLGERATINCRASESVDSSGNSFMHWYQQKPG |
| | QPPKLLIYRASNLESGVPDRESGSGSGTDFTLTISSLQAEDVAVY |
| | YCQQSNEDPWTFGGGTKLEIKRTVAAPSVFIFPPSDEQLKSGTAS |
| | VVCLLNNFYPREAKVQWKVDNALQSGNSQESVTEQDSKDSTYSLS |
| | STLTLSKADYEKHKVYACEVTHQGLSSPVTKSENRGEC |
| 15 | ATGGGATGGTCATGTATCATCCTTTTTCTGGTAGCAACTGCAACT |
| | GGAGTACATAGCCAGGTGCAGTTGGTACAATCGGGCGCCGAAGTG |
| | AAGAAACCAGGCGCCAGCGTCAAGGTCTCTTGTAAAGCATCTGGA |
| | TATACCTTCACCTCCTATTGGGTCGATTGGGTCCGCCAAGCCCCG |
| | GGACAGGGCCTGGAGTGGATGGGGAACATTTATCCAAGTGACTCT |
| | GAAACTCACTACAATCAGAAGTTCAAGGACAGAGTCACCATGACC |
| | CGAGATACAAGTACAAGCACAGTTTATATGGAGCTGAGTAGCCTG |
| | CGATCAGAGGACACAGCAGTCTATTACTGCGCTCGGAGCTACGGA |
| | TACTACGGTACTTGGTTTGCTTACTGGGGCCAGGGCACCTTGGTG |
| | ACAGTGTCCTCTGCTAGCACCAAGGGCCCATCGGTCTTCCCCCTG |
| | GCACCCTCCTCCAAGAGCACCTCTGGGGGCACAGCGGCCCTGGGC |
| | TGCCTGGTCAAGGACTACTTCCCCGAACCGGTGACGGTGTCGTGG |
| | AACTCAGGCGCCCTGACCAGCGGCGTGCACACCTTCCCGGCCGTC |
| | CTACAGTCCTCAGGACTCTACTCCCTCAGCAGCGTGGTGACCGTG |
| | CCCTCCAGCAGCTTGGGCACCCAGACCTACATCTGCAACGTGAAT |
| | CACAAGCCCAGCAACACCAAGGTGGACAAGCGGGTTGAGCCCAAA |
| | TCTTGTGACAAAACTCACACATGCCCACCGTGCCCAGCACCTGAA |
| | CTCCTGGGGGGACCGTCAGTCTTCCTCTTCCCCCCAAAACCCAAG |
| | GACACCCTCATGATCTCCCGGACCCCTGAGGTCACATGCGTGGTG |
| | GTGGACGTGAGCCACGAAGACCCTGAGGTCAAGTTCAACTGGTAC |
| | GTGGACGGCGTGGAGGTGCATAATGCCAAGACAAAGCCGCGGGAG |
| | GAGCAGTACGCCAGCACGTACCGTGTGGTCAGCGTCCTCACCGTC |
| | CTGCACCAGGACTGGCTGAATGGCAAGGAGTACAAGTGCAAGGTC |
| | TCCAACAAAGCCCTCCCAGCCCCCATCGAGAAAACCATCTCCAAA |
| | GCCAAAGGGCAGCCCCGAGAACCACAGGTGTACACCCTGCCCCCA |
| | TCCCGGGAGGAGATGACCAAGAACCAGGTCAGCCTGACCTGCCTG |
| | GTCAAAGGCTTCTATCCCAGCGACATCGCCGTGGAGTGGGAGAGC |
| | AATGGGCAGCCGGAGAACAACTACAAGACCACGCCTCCCGTGCTG |
| | GACTCCGACGGCTCCTTCTTCCTCTACAGCAAGCTCACCGTGGAC |
| | AAGAGCAGGTGGCAGCAGGGGAACGTCTTCTCATGCTCCGTGATG |
| | CATGAGGCTCTGCACAACCACTACACGCAGAAGAGCCTCTCCCTG |
| | TCTCCGGGCAAATAATAG |
| 16 | ATGGAGACAGACACACTCCTGCTATGGGTACTGCTGCTCTGGGTT |
| | CCAGGCTCCACCGGCGATATCGTGATGACACAATCTCCTGACTCC |
| | CTGGCCGTCAGCCTGGGAGAACGGGCCACAATTAATTGTCGTGCC |
| | TCTGAGAGCGTGGACTCTAGCGGCAACTCTTTCATGCACTGGTAT |
| | CAGCAAAAACCAGGACAGCCACCTAAGTTGCTGATCTACCGGGCT |
| | AGCAACCTCGAATCCGGAGTGCCCGATCGGTTTAGTGGCAGTGGC |
| | AGTGGCACTGACTTTACTCTGACCATCTCCTCGCTTCAAGCCGAG |
| | GATGTGGCTGTGTATTATTGTCAACAATCCAATGAGGATCCTTGG |
| | ACCTTTGGCGGTGGCACCAAGCTGGAGATCAAGCGTACGGTGGCT |
| | GCACCATCTGTCTTCATCTTCCCGCCATCTGATGAGCAGTTGAAA |
| | TCTGGAACTGCCTCTGTTGTGTGCCTGCTGAATAACTTCTATCCC |
| | AGAGAGGCCAAAGTACAGTGGAAGGTGGATAACGCCCTCCAATCG |
| | GGTAACTCCCAGGAGAGTGTCACAGAGCAGGACAGCAAGGACAGC |
| | ACCTACAGCCTCAGCAGCACCCTGACGCTGAGCAAAGCAGACTAC |
| | GAGAAACACAAAGTCTACGCCTGCGAAGTCACCCATCAGGGCCTG |
| | AGCTCGCCCGTCACAAAGAGCTTCAACAGGGGAGAGTGTTAATAG |

The pharmaceutical formulation of the present disclosure can comprise the monoclonal anti-OX40 antibodies as described above or antigen-binding fragments thereof at a concentration in a range of 0.5-200 mg/mi. In some embodiments, the concentration of the monoclonal anti-OX40 antibody or antigen-binding fragments thereof is any concentration value within the above range. For example, according to the need, the concentration of the monoclonal anti-OX40 antibody in the pharmaceutical formulation is about 1-180 mg/ml, about 10-160 mg/ml, about 15-140 mg/ml, about 20-120 mg/ml, about 25-100 mg/ml, about 30-80 mg/ml, about 40-60 mg/ml, or about 50 mg/ml.

In certain embodiments, the pharmaceutical formulation of the present disclosure can comprise the monoclonal anti-OX40 antibodies as described above or antigen-binding fragments thereof at a concentration of at least 0.5 mg/ml, at least 1 mg/ml, at least 2 mg/ml, at least 3 mg/ml, at least 4 mg/ml, at least 5 mg/ml, at least 6 mg/ml, at least 7 mg/ml, at least 8 mg/ml, at least 9 mg/ml, at least 10 mg/ml, at least 20 mg/ml, at least 30 mg/ml, at least 40 mg/ml, at least 50 mg/ml, at least 60 mg/ml, at least 70 mg/ml, at least 80 mg/ml, at least 90 mg/ml, at least 100 mg/ml, at least 120 mg/ml, at least 140 mg/ml, at least 160 mg/ml, or at least 180 mg/ml.

In certain embodiments, the pharmaceutical formulation of the present disclosure can comprise the monoclonal anti-OX40 antibodies as described above or antigen-binding fragments thereof at a concentration of at most 200 mg/ml, at most 180 mg/ml, at most 160 mg/ml, at most 140 mg/ml, at most 120 mg/ml, at most 100 mg/ml, at most 90 mg/ml, at most 80 mg/ml, at most 70 mg/ml, at most 60 mg/ml, at most 50 mg/ml, at most 40 mg/ml, at most 30 mg/ml, at most 20 mg, at most 10 mg/ml, at most 9 mg/ml, at most 8 mg/ml, at most 7 mg/ml, at most 6 mg/ml, at most 5 mg/ml, at most 4 mg/ml, at most 3 mg/ml, at most 2 mg/ml, or at most 1 mg/ml.

In certain embodiments, the pharmaceutical formulation of the present disclosure can include one or more monoclonal anti-OX40 antibodies as described above or antigen-binding fragments thereof. Concentrations of the monoclonal anti-OX40 antibodies as described above or antigen-binding fragments thereof can vary, for example, depending on various factors, such as the API activity, the mode of administration, the indications to be treated, the treatment regime and whether the pharmaceutical formulation is intended for long term storage in either lyophilized or liquid form. The skilled person in the art can readily determine the approximate concentration of the APIs without undue experimentation.

In some embodiments, the pharmaceutical formulation of the present disclosure may comprise the monoclonal anti-OX40 antibody at a concentration of about 40-60 mg/ml. In some embodiments, the pharmaceutical formulation of the present disclosure may comprise the monoclonal OX40 antibody at a concentration of about 35-55 mg/ml, about 40-50 mg/ml, or about 50 mg/ml. In some embodiments, the pharmaceutical formulation of the present disclosure may comprise the monoclonal OX40 antibody at a concentration of about 50 mg/ml.

### Method of Formulation Preparation

The present disclosure also provides a method of preparing the pharmaceutical formulations provided herein. The method can comprise combining a buffer solution having a pH from about 5.0 to about 8.0 (e.g., about 5.0 to about 5.5), a stabilizer, a surfactant, and a therapeutically effective amount of one or more APIs. One or more of the excipients in the pharmaceutical formulations described herein can be combined with therapeutically effective amounts of one or more APIs to produce a wide range of pharmaceutical formulations. The buffer, stabilizer, surfactant, and API have been described in the section “Formulation” above.

Generally, a pharmaceutical formulation should be sterilized, which can be achieved by either using sterile reagents in a sterile manufacturing environment or being sterilized following preparation. For example, sterile pharmaceutical formulations can be prepared by incorporating one or more APIs in the required amount into a buffer together with other excipients (e.g., stabilizer and surfactants) described herein, before applying a sterilization method, such as micro filtration.

In certain embodiments, the method of preparing the pharmaceutical formulations provided herein comprises a formulation process comprising:
1) preparing concentrated API (e.g., monoclonal anti-OX40 antibody or antigen-binding fragment thereof) at a concentration of 63±6 mg/ml (e.g., 47 mg/ml, 48 mg/ml, 49 mg/ml, 50 mg/ml, 51 mg/ml, 52 mg/ml, 53 mg/ml, 54 mg/ml, 55 mg/ml, 56 mg/ml, 57 mg/ml, 58 mg/ml, 59 mg/ml, 60 mg/ml, 61 mg/ml, 62 mg/ml, or 63 mg/ml) (from Ultrafiltration/Diafiltration, UF/DF) in 20 mM glutamic acid/histidine buffer at pH of 5.0;
2) adding sucrose to the concentrated API at the final concentration of 8% (w/v) from stock solution 40% (w/v);
3) adding polysorbate 80 to the UF/DF pool at the final concentration of 0.02% (w/v) from stock solution 10% (w/w) polysorbate 80;
4) filtering the formulated bulk through a 0.2 μm filter and then filling it into PC bottles. The sucrose stock solution 40% (w/w), and polysorbate 80 stock solution 10% (w/w) can be prepared in 20 mM glutamic acid & histidine at pH of 5.0.

In certain embodiments, the method of preparing the pharmaceutical formulations provided herein can further comprise a cell culture process and a purification process before the formulation process as described above. The cell culture process can be carried with vial thaw and seed culture expansion in shake flask, cell expansion in an RM bioreactor, cell expansion in an XDR 200 L bioreactor, production culture in a SUB 500 L bioreactor and depth filtration harvest. The purification process can be carried out using affinity chromatography (AC), low pH virus inactivation and neutralization (VIN), intermediate depth filtration (Int. DF), anion exchange (AEX) chromatography, cation exchange (CEX) chromatography, viral filtration (VF) and Ultrafiltration/Diafiltration (UF/DF).

Once a pharmaceutical formulation is prepared as described above, stability of the one or more APIs contained within the pharmaceutical formulation can be assessed using methods known in the art, such as those used in the Examples, including, without limitation, size exclusion chromatography, particle counting, anion/cation exchange chromatography, functional assays, such as binding activity.

### Kit

Also provided herein is a kit comprising one or more containers, each of which comprises one or more the excipients and the API(s) as described above. In certain embodiments, the kit comprises in one or more containers, a buffer such as glutamic acid & histidine buffer or aspartic acid & histidine buffer, a stabilizer such as sucrose, sorbitol or trehalose, a surfactant such as PS80, and a monoclonal anti-OX40 antibody or antigen-binding fragment thereof, as well as instructions regarding the use thereof.

In certain embodiments, the kits provided herein comprise one or more single or multi-chambered syringes, such as liquid syringes and lyosyringes, for administering the pharmaceutical formulations of the present disclosure. The kits provided herein can comprise a pharmaceutical formulation for human use.

### Uses

In another aspect, the present disclosure further provides a method of treating OX40-associated disease in a subject in need thereof, comprising administering a therapeutically effective amount of the pharmaceutical formulation of the present disclosure to the subject. As used herein, the term “OX40-associated disease” refers to a disease that requires treatment with OX40 agonist or antagonist, for example, cancer, or inflammation disease and/or autoimmune disease. The term “inflammation disease and/or autoimmune disease” as used herein refers to any inflammatory or immune-related conditions, such as pathological inflammation and autoimmune disease. The term “autoimmune disease”, as used herein, refers to a disease or condition that is caused by and target a subject's own tissue or organs, for example, generation of B cells that produce antibodies that recognizes normal body tissues and antigens may cause autoimmune diseases; secretion of autoantibodies specific to epitopes derived from self-antigens may also cause autoimmune disease.

In certain embodiments, the cancer is selected from the group consisting of breast cancer, melanoma, small-cell lung cancer, kidney cancer, stomach cancer, liver cancer, ovarian cancer, lymphatic leukemia myeloma, prostate cancer, urothelial cancer, head and neck cancer, non-small cell lung cancer, mesothelioma, skin cancer, lymphoma, leukemia, and sarcoma.

In certain embodiments, the inflammation disease and/or autoimmune disease is selected from the group consisting of idiopathic dermatitis, autoimmune uveitis, scleroderma, multiple sclerosis, lupus (e.g., systemic lupus erythematosus), rheumatoid arthritis, asthma (e.g., allergic asthma), chronic obstructive pulmonary disease (COPD), ulcerative colitis, and graft-versus-host disease (GVHD).

The pharmaceutical formulation of the present disclosure can be administered via one or more routes of administration using one or more of a variety of methods known in the art. As appreciated by the skilled artisan, the route and/or mode of administration vary depending upon the desired results. Preferred routes of administration include intravenous, intramuscular, intradermal, intraperitoneal, subcutaneous, spinal or other parenteral routes of administration, for example by injection or infusion. More preferred routes of administration are intravenous or subcutaneous. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the pharmaceutical formulation of the present disclosure can be administered via a non-parenteral route, such as a topical, epidermal or mucosal route of administration, for example, intranasally, orally, vaginally, rectally, sublingually or topically.

In some embodiments, the pharmaceutical formulation of the present disclosure is suitable for subcutaneous administration, or via intravenous infusion. In some embodiments, the pharmaceutical formulation of the present disclosure suitable for subcutaneous administration comprises the monoclonal anti-OX40 antibody or fragment thereof at a concentration of about 50 mg/mL.

The pharmaceutical formulation of the present disclosure can be administered at a single or multiple doses. As used herein, the term “dose” or “dosage” are interchangeable and indicates an amount of drug substance administered per body weight of a subject or a total dose administered to a subject irrespective to their body weight.

In some embodiments, a therapeutically effective amount of the pharmaceutical formulation of the present disclosure is administrated to a patient in need thereof. As used herein, the term “therapeutically effective amount” refers to the minimum concentration required to achieve a detectable improvement or prevention of a particular disease or condition. The ability of a pharmaceutical formulation to improve or prevent a particular disease or condition can be evaluated, for example, in an animal model system or an in vitro system, predictive of efficacy for the targeted disease or condition in human. Actual dosage of the APIs in the pharmaceutical formulations of the present disclosure can be determined based on various factors, such as the size and location of the area to be treated, subject's body weight, the severity of the subject's symptoms, the nature of the selected APIs (e.g., whole antibody or fragment), the mode of administration, and any additional agents administered before, at the time of or after administration of the APIs in the pharmaceutical formulations of the present disclosure.

In yet another aspect, the present disclosure provides use of the pharmaceutical formulation in manufacture of a medicament for the treatment of an OX40-associated disease.

### Other Embodiments of the Formulations

Embodiment 1. A pharmaceutical formulation comprising:
an antibody or antigen-binding fragment,
a buffer selected from glutamic acid & histidine buffer or aspartic acid & histidine buffer,
a stabilizer selected from sucrose, sorbitol or trehalose at a concentration of about 0.5% to about 50% (w/v),
a surfactant that is polysorbate 80 at a concentration of about 0.001% to about 0.1% (w/v),
wherein the pharmaceutical formulation has a pH of about 5.0 to about 8.0.

Embodiment 2. The pharmaceutical formulation of embodiment 1, wherein the buffer is present at a concentration of about 1-100 mmol/L, about 10-90 mmol/L, about 10-80 mmol/L, about 10-70 mmol/L, about 10-60 mmol/L, about 10-50 mmol/L, about 10-40 mmol/L, about 10-30 mmol/L, or about 20 mmol/L.

Embodiment 3. The pharmaceutical formulation of embodiment 1 or 2, wherein the buffer is present at a concentration of about 10-30 mmol/L.

Embodiment 4. The pharmaceutical formulation of any one of preceding embodiments, wherein the buffer is at a concentration of about 20 mmol/L.

Embodiment 4. The pharmaceutical formulation of any one of preceding embodiments, wherein the pharmaceutical formulation has a pH of about 5.0 to about 6.0.

Embodiment 5. The pharmaceutical formulation of any one of preceding embodiments, wherein the pharmaceutical formulation has a pH of about 5.0 to about 5.5.

Embodiment 6. The pharmaceutical formulation of any one of preceding embodiments, wherein the pharmaceutical formulation has a pH of about 5.0.

Embodiment 7. The pharmaceutical formulation of any one of preceding embodiments, wherein the stabilizer is present at a concentration of about 0.5% to about 50% (w/v), about 1% to about 40% (w/v), about 2% to about 30% (w/v), about 3% to about 20% (w/v), about 3.2% to about 18% (w/v), about 3.4% to about 16% (w/v), about 3.6% to about 14% (w/v), about 4% to about 12% (w/v), about 6% to about 10% (w/v), or about 8% (w/v).

Embodiment 8. The pharmaceutical formulation of any one of preceding embodiments, wherein the stabilizer is sucrose at a concentration of about 4% to about 12% (w/v).

Embodiment 9. The pharmaceutical formulation of any one of preceding embodiments, wherein the stabilizer is sucrose at a concentration of about 8% (w/v).

Embodiment 10. The pharmaceutical formulation of any one of embodiments 1-7, wherein the stabilizer is sorbitol at a concentration of about 0.5%-10% (w/v).

Embodiment 11. The pharmaceutical formulation of any one of embodiments 1-7, wherein the stabilizer is sorbitol at a concentration of about 4.5% (w/v).

Embodiment 12. The pharmaceutical formulation of any one of embodiments 1-7, wherein the stabilizer is trehalose at a concentration of about 4%-14% (w/v).

Embodiment 13. The pharmaceutical formulation of any one of embodiments 1-7, wherein the stabilizer is trehalose at a concentration of about 8.8% (w/v).

Embodiment 14. The pharmaceutical formulation of any one of preceding embodiments, wherein the polysorbate 80 is present at a concentration of about 0.002% to about 0.08% (w/v), about 0.004% to about 0.06% (w/v), about 0.006% to about 0.05% (w/v), about 0.008% to about 0.05% (w/v), about 0.01% to about 0.05% (w/v), or about 0.02% (w/v).

Embodiment 15. The pharmaceutical formulation of any one of preceding embodiments, wherein the polysorbate 80 is present at a concentration of about 0.01% to about 0.04% (w/v), about 0.01% to about 0.03% (w/v), or about 0.02% (w/v).

Embodiment 16. The pharmaceutical formulation of any one of preceding embodiments, wherein the polysorbate 80 is at a concentration of about 0.02% (w/v).

Embodiment 17. The pharmaceutical formulation of any one of preceding embodiments, wherein the antibody or antigen-binding fragment is present at a concentration of about 0.5-200 mg/ml, about 1-180 mg/ml, about 10-160 mg/ml, about 15-140 mg/ml, about 20-120 mg/ml, about 25-100 mg/ml, about 30-80 mg/ml, about 40-60 mg/ml, or about 50 mg/ml.

Embodiment 18. The pharmaceutical formulation of any one of preceding embodiments, wherein the antibody or antigen-binding fragment is present at a concentration of about 45-55 mg/ml, or about 50 mg/ml.

Embodiment 19. The pharmaceutical formulation of any one of preceding embodiments, wherein the antibody or antigen-binding fragment is present at a concentration of about 50 mg/ml.

Embodiment 20. The pharmaceutical formulation of any one of preceding embodiments, wherein the antibody or antigen-binding fragment is a monoclonal anti-OX40 antibody or antigen-binding fragment thereof.

Embodiment 21. The pharmaceutical formulation of embodiment 20, wherein the monoclonal anti-OX40 antibody comprises a heavy chain and a light chain, wherein the heavy chain comprises a heavy chain variable region V_{H} which comprises:
HCDR1 comprising the amino acid sequence as set forth in SEQ ID NO: 1;
HCDR2 comprising the amino acid sequence as set forth in SEQ ID NO: 2,
HCDR3 comprising the amino acid sequence as set forth in SEQ ID NO: 3;
   wherein the light chain comprises a light chain variable region V_{L} which comprises:
LCDR1 comprising the amino acid sequence as set forth in SEQ ID NO: 4,
LCDR2 comprising the amino acid sequence as set forth in SEQ ID NO: 5,
LCDR3 comprising the amino acid sequence as set forth in SEQ ID NO: 6.

Embodiment 22. The pharmaceutical formulation of embodiment 21, wherein the monoclonal anti-OX40 antibody further comprises Fc region variant.

Embodiment 23. The pharmaceutical formulation of embodiment 22, wherein the Fc region variant is human IgG1 N297A.

Embodiment 24. The pharmaceutical formulation of any one of preceding embodiments, wherein the heavy chain variable region V_{H} comprises the amino acid sequence selected from the group consisting of: SEQ ID NO: 7, SEQ ID NO: 9, and SEQ ID NO: 10.

Embodiment 25. The pharmaceutical formulation of any one of embodiments 20-24, wherein the light chain variable region V_{L} comprises the amino acid sequence selected from the group consisting of: SEQ ID NO: 8, SEQ ID NO: 11, and SEQ ID NO: 12.

Embodiment 26. The pharmaceutical formulation of any one of embodiments 20-25, wherein the pair of heavy chain variable region V_{H} and light chain variable region V_{L} is selected from the group consisting of: SEQ ID NO: 7 and SEQ ID NO: 8, SEQ ID NO: 9 and SEQ ID NO: 11, SEQ ID NO: 10 and SEQ ID NO: 8, SEQ ID NO: 7 and SEQ ID NO: 11, and SEQ ID NO: 10 and SEQ ID NO: 12.

Embodiment 27. The pharmaceutical formulation of any one of embodiments 1 to 26, wherein the pharmaceutical formulation is suitable for subcutaneous administration or for intravenous administration.

Embodiment 28. Use of the pharmaceutical formulation of any one of embodiments 1 to 27 in the manufacture of a medicament for the treatment or prevention of OX40-associated disease.

Embodiment 29. The use of embodiment 28, wherein the OX40-associated disease is inflammation and/or autoimmune diseases (such as graft-versus-host disease).

Embodiment 30. A method of treating OX40-associated disease in a subject in need thereof, comprising administering a therapeutically effective amount of the pharmaceutical formulation of any one of embodiments 1 to 27 to the subject.

Embodiment 31. The method of embodiment 30, wherein the administration is via subcutaneous injection or intravenous injection.

### Examples

The present disclosure can be better understood with reference to the following examples. However, the following examples are intended to illustrate the present disclosure and should not be understood as limiting the scope of the present disclosure. Various changes and modifications may be made in light of the teachings herein, and thus such changes and modifications are within the scope of the present disclosure.

This study reported the solubility profiling study, pH/buffer screening, and excipient screening and surfactant strength screening for the pharmaceutical formulation of the present disclosure.

The solubility profiling study investigated acetate buffer, citrate/arginine buffer and arginine buffer via PEG concentration study and Tagg. The stability of the formulation was investigated up to 4-week incubation in 9 different pH/buffer conditions at temperature of 40±2° C. and relative humidity of 75±5%.

In excipient screening and surfactant strength screening, 7 different formulations were investigated under several conditions, i.e., in incubation at 40° C. for 2 or 4 weeks, freeze-thaw from −70° C. to room temperature for 3 or 5 cycles and agitation at 300 rpm for 1 or 3 days.

### Example 1: Analytical Methods

### Appearance

Cleaned the outer wall of the glass vials, then held the neck of glass vials close to the edge of the gobo of YB-2 lightbox at a distance of 25 cm away. The appearance of samples, including color, clarity, and visible particles, were examined against black and white background with an illumination level at 2000-3750 Lux.

### Peg Concentration Study

Final PEG concentration is set up from 2.5% to 15%, 50 μL PEG stock solution were added into 50 μL sample solution containing protein and mixed well. Then the protein concentration was detected by Nanodrop. The curve of PEG concentration versus protein concentration was then obtained.

### Protein Concentration

After the samples were mixed evenly, protein concentration was determined by UV280 readings using a NanoDrop 2000 spectrophotometer. The extinction coefficient was 1.62 AU*mL*mg⁻¹*cm⁻¹. All measurements were repeated twice at a loading volume of 2.5 μL each time and an average was taken.

pH

The pH meter was calibrated with three different standard buffers (pH 4.01, 7.00 and 9.21) prior to use. The slope of calibration was between 95.0%-105.0%, and the zero drift was between −60.0 mv-+60.0 mv. After that, pH was measured at a loading volume of 50 μL for each sample.

### Osmolality

The osmolality of 20 μL undiluted samples was measured by Advanced 2020 Osmometer for twice, and an average was taken. Before and after the measurement, the osmometer was calibrated by 290 mOsm reference solution.

### DSC

DSC is a thermoanalytical technique in which the difference in the amount of heat required to increase the temperature of a sample and the reference is measured as a function of temperature. Samples were diluted to 1 mg/mL with its reference buffer. 400 μL of respective reference buffers were added into the odd-numbered wells of a 96-well plate and 400 μL of samples were added into the even-numbered wells of the same plate. Experimental parameters were set such that the scan temperature ramped from 10-95° C. with a rate of 90° C./h. Analysis of thermograms was performed in the MicroCal PEAQ DSC automated data analysis software.

### Caliper-SDS

Caliper-SDS is a purity method utilized to determine the truncation or fragmentation level of the biomolecules during the production and storage processes. Caliper-SDS employs a microchip to separate proteins based on their electrophoretic mobility, with proteins of smaller sizes move faster and of larger sizes move slower in a capillary.

In this study, the samples were firstly diluted to 1.0 mg/mL with ultrapure water, and then mixed with the denaturing solution, composed of sample buffer, SDS and N-ethylmaleimide (for the non-reduced method) or dithiothreitol (for the reduced method). The mixtures were incubated at 70° C. for 10 mins, and then transferred into a 96-well plate. After the plate was loaded onto the plate holder of the instrument, samples were sipped, stained, separated and detected in the microchip. Data were acquired with LabChip GX Reviewer.

The percentage of the main peak was reported as the purity of the sample in the non-reduced Caliper-SDS (Caliper-SDS-NR). While for the reduced method (Caliper-SDS-R), the percentage of the sum of LC+HC (purity) was reported.

### SEC-HPLC

SEC-HPLC was used to provide information concerning stability of the protein as measured by aggregation of the protein under certain conditions, e.g., during storage. The data can be presented as a percentage of the main peak (monomer), and greater main peak percentage means less aggregation (e.g., dimer and other high molecular weight aggregation) of the protein.

SEC was performed on an Agilent HPLC system with a SEC column (300×7.8 mm, 5 μm). The sampler temperature was set to 5±3° C. and the column oven temperature was set as 25±3° C. The mobile phase was 50 mM PB, 300 mM NaCl, pH 6.8±0.1 and the flow rate was set as 1.0 mL/min. 100 g of each sample was injected. Detection wavelength was set at 280 nm and the run time was 20 minutes.

iCIEF

Imaged capillary isoelectric focusing (iCIEF) is a purity method used to monitor the charge variant species by determining the isoelectric point (pI) and distribution of each variant.

Charge variants of the protein are separated based on their unique pI, which is an intrinsic property of a specific protein and is the pH at which the protein molecule does not carry any net charge. Under an external electric field, the charge variants move along a continuous pH gradient formed by the ampholytes and stop at the position where the pH equals to its pI.

In this study, protein samples were firstly diluted to 1.0 mg/mL with ultrapure water. 20 μL diluted sample was then mixed with 80 μL master mix, composed of pI markers 4.65/9.22, carrier ampholytes (3-10), methyl cellulose and urea, before being loaded into a capillary for electric focusing by Capillary Isoelectric Focusing System.

The percentage of the main peak, acidic peaks and basic peaks was reported in the final results, along with the pI of the main peak. Acidic peaks stand for the acidic species, which are defined as the antibody variants that elute earlier than the main peak during cation-exchange chromatography (CEX) or later than the main peak during anion exchange chromatography (AEX) analysis. Acid species can be formed through modifications including sialic acid, deamidation, non-classical disulfide linkage, trisulfide bonds, high mannose, glycation, modification by maleuric acid, cysteinylation, reduced disulfide bonds, non-reduced species and/or fragments. Basic peaks stand for basic species, which are defined as the materials that elute later than the main peak during CEX and earlier than the main peak during AEX analyses. Basic species can be formed through modifications including C-terminal Lys, N-terminal Glu, Isomerization of Asp, Succinimide, Met oxidation, Amidation, Incomplete disulfide bonds, Incomplete removal of leader sequence, Mutation from Ser to Arg, glycosylation, Fragments and/or Aggregates. Main peak refers to the main species, which stands for the target antibody molecule that elutes as the major peak on chromatograms. The main species does not necessarily correspond to the unmodified or non-degraded antibody. In fact, the main peak typically consists of species of antibodies with three types of typical post-translational modifications: (1) cyclization of the N-terminal glutamine (Gln) to pyroGlu; (2) removal of the heavy chain C-terminal lysine (Lys); and (3) glycosylation of the conserved asparagine (Asn) residue in the C_{H2} domain with neutral oligosaccharides.

### MFI

Sub-visible particles were monitored by an MFI system 5200. 1.5 mL volume of each sample was transferred to the MFI 96-well plate in bio-safety hood for analysis. The results were analyzed by the vendor's software. The amount of sub-visible particle in the equivalent circular diameter ≥2 μm, ≥5 μm, ≥10 μm and ≥25 μm was reported.

### Tagg (Aggregation Temperature)

Tagg is a thermal analysis parameter used to predict the thermodynamic stability of proteins, which can be characterized by dynamic light scattering (DLS) method.

Measurements were performed on Wyatt DynaPro Plate Reader II. Before experiment, purge both sides of 384 well plate with clean nitrogen to keep it clean, then add 20 μL sample into corresponding position, centrifuge for 5 min with the speed of 4000 rpm, and finally drop 15 μL paraffin oil on the sample for liquid sealing. During the detection, samples were heated from 25° C. to 75° C., and the data analysis was completed by the software provided by the equipment manufacturer.

kD

kD is a thermal analysis parameter used to predict the thermodynamic stability of proteins, which can be characterized by DLS method. Measurements were performed on Wyatt DynaPro Plate Reader II. Before experiment, purge both sides of 384 well plate with clean nitrogen to keep it clean, then add 20 μL sample into corresponding position, centrifuge for 5 min with the speed of 4000 rpm, and finally drop 15 μL paraffin oil on the sample for liquid sealing. During the detection, samples were incubated at 20° C., 25° C., 30° C., 35° C. and 40° C., and the data analysis was completed by the software provided by the equipment manufacturer.

### Example 2: Solubility Profiling

### Purpose

This study aimed to understand the relationship between solubility, pH and ionic strength for pH/buffer screening.

### Material

The anti-OX40 antibody Drug Substance (DS) has a heavy chain of SEQ ID NO: 13 and a light chain of SEQ ID NO: 14.

### Experiment Method

The anti-OX40 antibody Drug Substance (DS) was exchanged to Acetate buffer (a buffer system consisting of, essentially, acetate and sodium acetate), Citrate/Arginine buffer (a buffer system consisting of, essentially, citric acid and arginine) and Arginine buffer (a buffer system consisting of, essentially, arginine and arginine-HCl) via ultra-centrifugal filter. The protein concentration at 2 mg/mL was filtered through 0.22 μm PVDF syringe filter. Set up the PEG concentration from 2.5% to 15%, then 50 μL sample was mixed with 50 μL PEG well. After that the protein concentration was determined by UV280. 8% Sucrose was added to each sample for Tagg testing. The study plan was shown in Table 2.

TABLE 2
The study plan of solubility profiling

| | |
|---|---|
| Buffer | Test |

| | | |
|---|---|---|
| 20 mM Acetate | pH 4.5 | Tagg, PEG |
| | pH 5.5 | |
| 200 mM Citrate/ | pH 5.5 | |
| Arginine | pH 6.5 | |
| | pH 7.5 | |

| | |
|---|---|
| 20 mM Arginine | pH 7.5 |
| | pH 8.5 |

### Results

### PEG

As shown in Table 3 and FIG. 1, no obvious change in protein concentration in acetate buffer pH 4.5 was observed. The protein concentration in arginine buffer decreased when the PEG concentration was 10%.

TABLE 3
Results of protein concentration in solubility profiling study

| | PEG Concentration % | | | | | |
|---|---|---|---|---|---|---|
| | 2.5 | 5 | 7.5 | 10 | 12.5 | 15 |
| Formulation Information | Protein Concentration % | | | | | |
| 20 mM Acetate pH 4.5 | 0.80 | 0.90 | 0.94 | 0.95 | 0.94 | 0.86 |
| 20 mM Acetate pH 5.0 | 0.91 | 0.94 | 1.01 | 0.92 | 0.37 | 0.05 |
| 200 mM Citrate/Arginine | 1.03 | 1.02 | 1.06 | 1.04 | 0.26 | 0.05 |
| pH 5.5 | | | | | | |
| 200 mM Citrate/Arginine | 0.90 | 0.91 | 0.98 | 0.93 | 0.48 | 0.08 |
| pH 6.5 | | | | | | |
| 200 mM Citrate/Arginine | 1.04 | 1.07 | 1.11 | 1.07 | 0.66 | 0.12 |
| pH 7.5 | | | | | | |
| 20 mM Arginine pH 7.5 | 1.03 | 1.01 | 0.87 | 0.16 | 0.07 | 0.03 |
| 20 mM Arginine pH 8.5 | 1.01 | 1.07 | 0.97 | 0.71 | 0.11 | 0.03 |

### Tagg

As shown in Table 4, the Tagg temperature of anti-OX40 antibody was 59.1° C., 60.3° C., 58.7° C. and 59.0° C. at pH 4.5, 5.0, 8.0 and 8.5, respectively.

TABLE 4
Results of Tagg in solubility profiling study

| Formulation Information | Tagg ° C. |
|---|---|
| 20 mM Acetate pH 4.5, 8% Sucrose | 59.1 |
| 20 mM Acetate pH 5.0, 8% Sucrose | 60.3 |
| 20 mM Acetate pH 5.5, 8% Sucrose | 50.7 |
| 200 mM Citrate/Arginine pH 5.5 | 46.9 |
| 200 mM Citrate/Arginine pH 6.5 | 45.2 |
| 200 mM Citrate/Arginine pH 7.5 | 45.4 |
| 20 mM Arginine pH 7.5, 8% Sucrose | 48.9 |
| 20 mM Arginine pH 8.0, 8% Sucrose | 58.7 |
| 20 mM Arginine pH 8.5, 8% Sucrose | 59.0 |

### Summary

Based on the results of solubility profiling study, the solubility of anti-OX40 antibody was the best in acetate buffer at pH 4.5 than that in other buffers, and anti-OX40 antibody has a higher aggregation temperature at lower ionic strength.

### Example 3: pH/Buffer Screening Study

### Purpose

This study aimed to pH/buffer screening for optimal protein storage.

### Material

The anti-OX40 antibody Drug Substance (DS) has a heavy chain of SEQ ID NO: 13 and a light chain of SEQ ID NO: 14. The acetate buffer is a buffer system consisting of, essentially, acetate and sodium acetate, the glutamic/histidine buffer is a buffer system consisting of, essentially, glutamic acid and histidine, and the arginine buffer is a buffer system consisting of, essentially, arginine and arginine-HCl, optionally with an acid (e.g., HCl) or a base (e.g., NaOH) for adjusting the final pH.

### Experiment Method

The anti-OX40 antibody DS was exchanged to acetate, glutamic/histidine and arginine buffer via ultrafiltration and diafiltration (UF/DF). The protein at 50 mg/mL was filtered through 0.22 μm PVDF syringe filter and then aliquoted to 2R vials (50 mg/vial). The vials were stoppered and capped and subjected to stability study under 40° C. as shown in Table 5.

TABLE 5
The study plan of pH/buffer screening

| | | | | 40° C. | | |
|---|---|---|---|---|---|---|
| | | | | 1 week | 2 weeks | 4 weeks |
| Buffer | | | T0 | (1 w) | (2 w) | (4 w) |
| F1 | 20 mM | pH 4.5 | X.Y | X | X | X, Z |
| F2 | Acetate | pH 5.0 | | | | |
| F3 | | pH 5.5 | | | | |
| F4 | 20 mM | pH 4.5 | | | | |
| F5 | Glutamic/ | pH 5.0 | | | | |
| F6 | Histidine | pH 5.5 | | | | |
| F7 | 20 mM | pH 8.0 | | | | |
| F8 | Argine | pH 8.5 | | | | |

X = Appearance, pH, protein concentration, SEC-HPLC, iCIEF, Caliper-SDS(R&NR)
Y = DSC
Z = Potency (only for the selected formulation)

### Results

### DSC

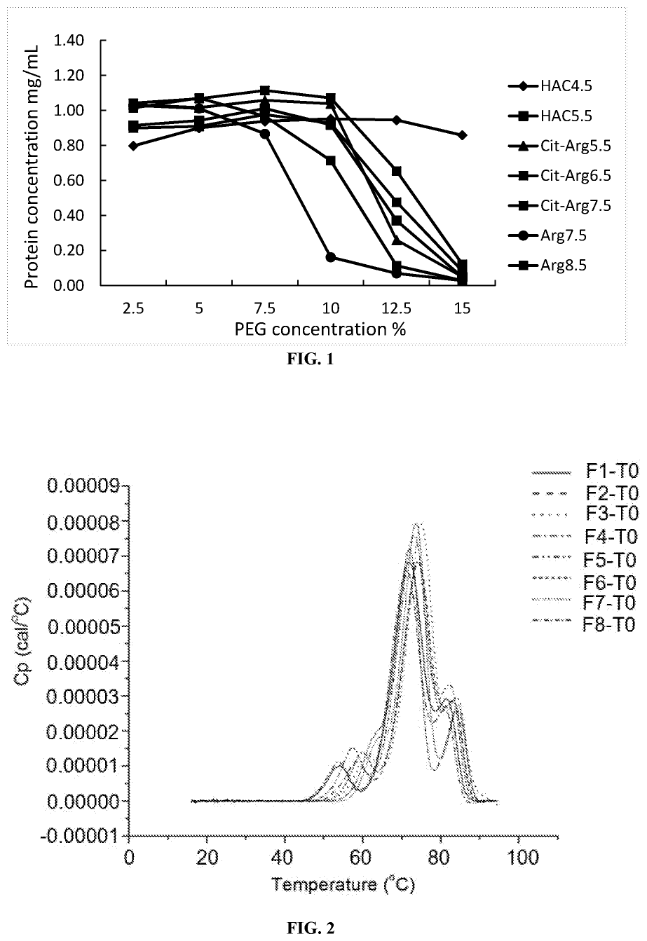

The DSC results were shown in Table 6 and FIG. 2. The Tmonset of the anti-OX40 antibody DS in arginine buffer pH 8.0 (57.0° C.) was higher than that in other buffers. The Tmonset of the anti-X40 antibody DS in acetate buffer was close to that in glutamic/histidine buffer. These results indicated that the thermal stability of the anti-OX40 antibody DS was worse at low pH. Tmonset stands for the temperature under which the protein starts to unfold and indicates the temperature at which the protein first domain start unfolding. Tm1 stands for thermal transition midpoint and indicates the temperature at which the first protein domain is half unfolded.

TABLE 6
Results of DSC in pH/buffer screening

| Formulation Information | | | Tm Onset ° C. | Tm1° C. |
|---|---|---|---|---|
| F1 | 20 mM | pH 4.5 | 45.3 | 54.2 |
| F2 | Acetate | pH 5.0 | 49.1 | 58.6 |
| F3 | | pH 5.5 | 53.7 | 75.0 |
| F4 | 20 mM | pH 4.5 | 44.5 | 54.4 |
| F5 | Glutamic/ | pH 5.0 | 48.1 | 57.2 |
| F6 | Histidine | pH 5.5 | 50.9 | 60.1 |
| F7 | 20 mM Argine | pH 8.0 | 57.0 | 73.7 |
| F8 | | pH 8.5 | 55.5 | 72.0 |

### Appearance, pH and Protein Concentration

The appearance, pH and protein concentration were shown in Table 7. Visible particles were observed in all buffers after 4 weeks. Only in arginine buffer, the particles were observed after 1 week. No obvious changes in color, pH or protein concentration were observed.

TABLE 7
Results of appearance, pH and protein concentration in pH/buffer screening

| | Time | | | UV280 |
|---|---|---|---|---|
| Sample name | point | Appearance | pH | (mg/mL) |
| 20 mM Acetate pH 4.5 | T0 | Slight yellow, slight opalescence, free | 4.6 | 48.9 |
| | | of particles | | |
| | 40° C. 1 W | Slight yellow, slight opalescence, free | 4.6 | 48.6 |
| | | of particles | | |
| | 40° C. 2 W | Slight yellow, slight opalescence, free | 4.6 | 48.5 |
| | | of particles | | |
| | 40° C. 4 W | Slight yellow, slight opalescence, | 4.6 | 48.5 |
| | | essentially free of particles | | |
| 20 mM Acetate pH 5.0 | T0 | Slight yellow, slight opalescence, free | 5.1 | 47.8 |
| | | of particles | | |
| | 40° C. 1 W | Slight yellow, slight opalescence, free | 5.1 | 47.5 |
| | | of particles | | |
| | 40° C. 2 W | Slight yellow, slight opalescence, free | 5.1 | 47.5 |
| | | of particles | | |
| | 40° C. 4 W | Slight yellow, slight opalescence, | 5.1 | 47.6 |
| | | essentially free of particles | | |
| 20 mM Acetate pH 5.5 | T0 | Slight yellow, slight opalescence, free | 5.5 | 50.2 |
| | | of particles | | |
| | 40° C. 1 W | Slight yellow, slight opalescence, free | 5.4 | 50.8 |
| | | of particles | | |
| | 40° C. 2 W | Slight yellow, slight opalescence, | 5.5 | 49.5 |
| | | essentially free of particles | | |
| | 40° C. 4 W | Slight yellow, slight opalescence, | 5.5 | 49.7 |
| | | essentially free of particles | | |
| 20 mM | T0 | Slight yellow, slight opalescence, free | 4.6 | 50.6 |
| Glutamic/Histidine pH | | of particles | | |
| 4.5 | 40° C. 1 W | Slight yellow, slight opalescence, free | 4.6 | 50.8 |
| | | of particles | | |
| | 40° C. 2 W | Slight yellow, slight opalescence, free | 4.6 | 50.2 |
| | | of particles | | |
| | 40° C. 4 W | Slight yellow, slight opalescence, | 4.6 | 50.3 |
| | | essentially free of particles | | |
| 20 mM | T0 | Slight yellow, slight opalescence, free | 5.2 | 49.0 |
| Glutamic/Histidine pH | | of particles | | |
| 5.0 | 40° C. 1 W | Slight yellow, slight opalescence, free | 5.1 | 48.7 |
| | | of particles | | |
| | 40° C. 2 W | Slight yellow, slight opalescence, free | 5.1 | 48.7 |
| | | of particles | | |
| | 40° C. 4 W | Slight yellow, slight opalescence, | 5.2 | 48.8 |
| | | essentially free of particles | | |
| 20 mM | T0 | Slight yellow, slight opalescence, free | 5.6 | 49.5 |
| Glutamic/Histidine pH | | of particles | | |
| 5.5 | 40° C. 1 W | Slight yellow, slight opalescence, free | 5.5 | 49.4 |
| | | of particles | | |
| | 40° C. 2 W | Slight yellow, slight opalescence, free | 5.5 | 49.4 |
| | | of particles | | |
| | 40° C. 4 W | Slight yellow, slight opalescence, | 5.5 | 49.6 |
| | | essentially free of particles | | |
| 20 mM Arginine pH 8.0 | T0 | Slight yellow, opalescence, free of | 8.0 | 50.1 |
| | | particles | | |
| | 40° C.1 W | Slight yellow, opalescence, | 7.9 | 50.3 |
| | | essentially free of particles | | |
| | 40° C.2 W | Slight yellow, opalescence, | 7.9 | 51.0 |
| | | essentially free of particles | | |
| | 40° C.4 W | Slight yellow, opalescence, | 7.8 | 50.7 |
| | | essentially free of particles | | |
| 20 mM Arginine pH 8.5 | T0 | Slight yellow, opalescence, free of | 8.4 | 49.2 |
| | | particles | | |
| | 40° C. 1 W | Slight yellow, opalescence, | 8.4 | 49.0 |
| | | essentially free of particles | | |
| | 40° C. 2 W | Slight yellow, opalescence, | 8.3 | 48.7 |
| | | essentially free of particles | | |
| | 40° C. 4 W | Slight yellow, opalescence, | 8.3 | 49.0 |
| | | essentially free of particles | | |

### SEC-HPLC

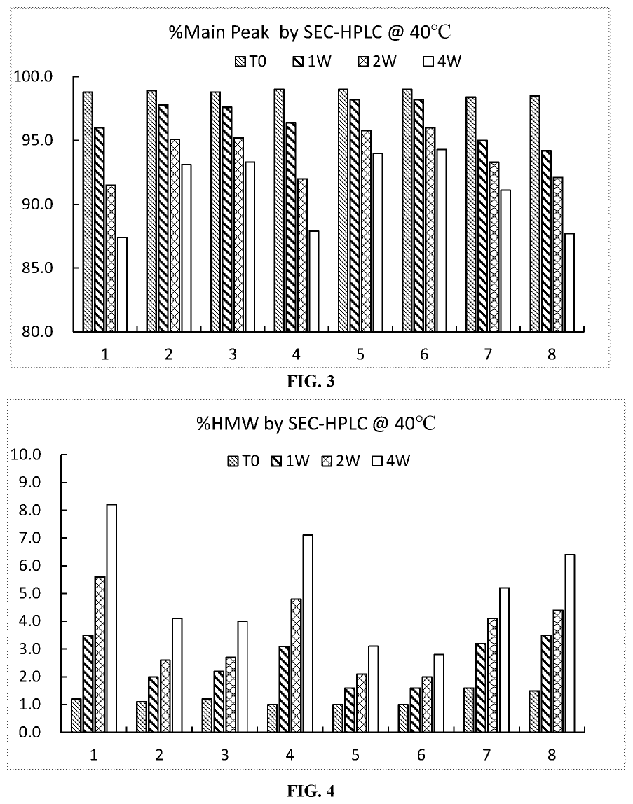

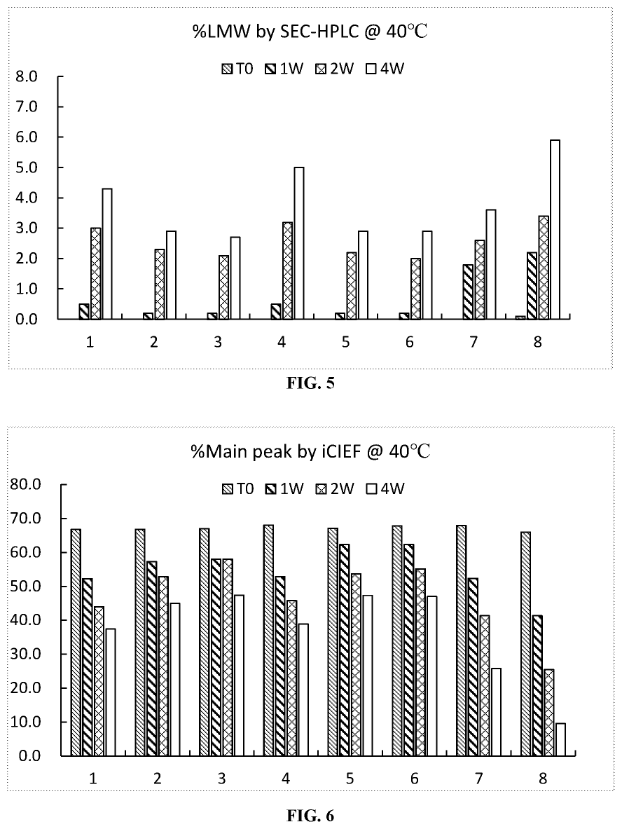

SEC-HPLC results were summarized in Table 8 while the compositions of main peak, aggregation and fragment were presented in FIG. 3, FIG. 4 and FIG. 5. The main peak in pH 4.5 showed the largest drop by 11.4% and 11.1% in acetate buffer and glutamic/histidine buffer, respectively. Whereas HMW peak increased by 7.0% and 6.1%, the LMW peak increased by 4.3% and 5.0%. The main peak in glutamic/histidine buffer showed the slightest drop by 4.7% at pH 5.5.

iCIEF

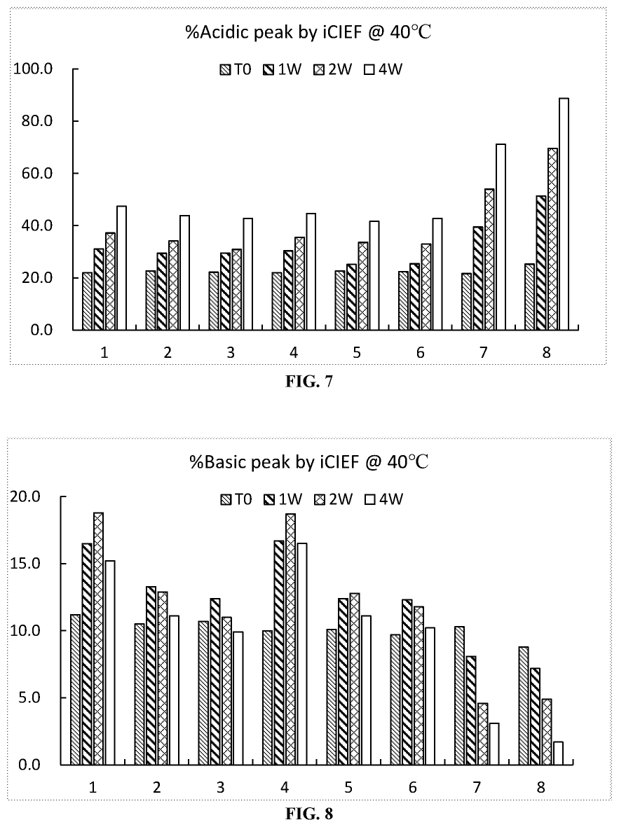

iCIEF results were summarized in Table 8 and the compositions of main peak, acidic peak and basic peak were shown in FIG. 6, FIG. 7 and FIG. 8. The main peak in arginine buffer showed the most drop by 56.4% at pH 8.5. The main peak decreased by 29.4% and 29.2% in acetate and glutamic/histidine buffer at pH 4.5, respectively.

### Caliper-SDS (Reduce&Non-reduce) (Caliper-SDS-R & Caliper-SDS-NR)

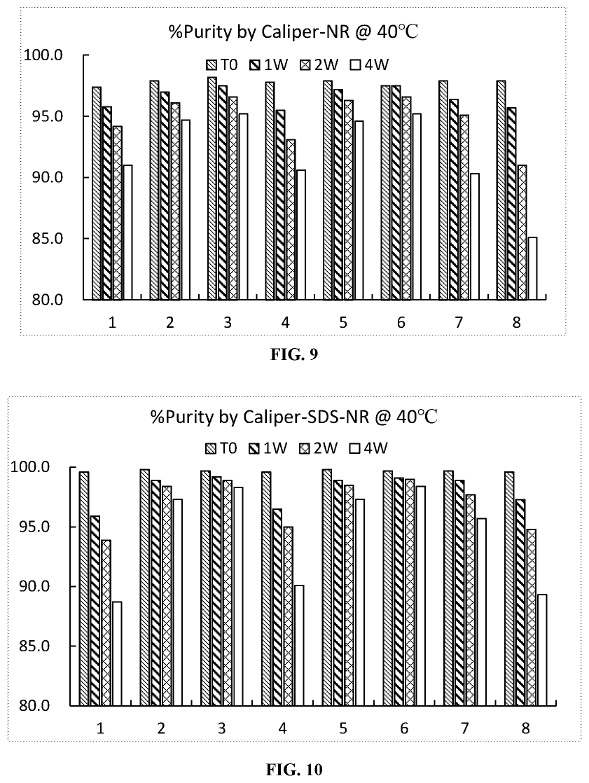

Caliper-SDS (Reduce&Non-reduce) results were summarized in Table 8 and the changes in the purity of the anti-OX40 antibody DS were presented in FIG. 9 and FIG. 10. The purity of non-reduced protein in arginine buffer at pH 8.5 dropped by 12.8%. The purity of non-reduced protein in acetate and glutamic/histidine buffer at pH 5.0 and 5.5 dropped by around 3%, while that in other pH conditions dropped by around 7%.

The purity of reduced protein dropped by 2.5% and 1.4% in acetate buffer at pH 5.0 and 5.5, which is close to that in glutamic/histidine buffer. In acetate buffer at pH 4.5 and arginine buffer at pH 8.5, the results of reduced protein were showed the most drop by 10.9% and 10.3%, respectively.

TABLE 8
The results of SEC-HPLC, iCIEF and Caliper-SDS(R&NR) in pH/buffer screening study

| | | | SEC-HPLC | | | iCIEF | | | Caliper- | Caliper- |
|---|---|---|---|---|---|---|---|---|---|---|
| | | | | Main | | Acidic | Main | Basic | SDS-NR | SDS-R |
| No | Sample name | Time point | HMW % | peak % | LMW % | peak % | peak % | peak % | Purity % | LC % + HC % |
| F1 | 20 mM Acetate pH | T0 | 1.2 | 98.8 | ND | 22.0 | 66.8 | 11.2 | 97.4 | 99.6 |
| | 4.5 | 40° C. 1 w | 3.5 | 96.0 | 0.5 | 31.1 | 52.3 | 16.5 | 95.8 | 95.9 |
| | | 40° C. 2 w | 5.6 | 91.5 | 3.0 | 37.2 | 44.0 | 18.8 | 94.2 | 93.9 |
| | | 40° C. 4 w | 8.2 | 87.4 | 4.3 | 47.4 | 37.4 | 15.2 | 91.0 | 88.7 |
| F2 | 20 mM Acetate pH | T0 | 1.1 | 98.9 | ND | 22.7 | 66.8 | 10.5 | 97.9 | 99.8 |
| | 5.0 | 40° C. 1 w | 2.0 | 97.8 | 0.2 | 29.4 | 57.3 | 13.3 | 97.0 | 98.9 |
| | | 40° C. 2 w | 2.6 | 95.1 | 2.3 | 34.2 | 52.9 | 12.9 | 96.1 | 98.4 |
| | | 40° C. 4 w | 4.1 | 93.1 | 2.9 | 43.9 | 45.0 | 11.1 | 94.7 | 97.3 |
| F3 | 20 mM Acetate pH | T0 | 1.2 | 98.8 | ND | 22.2 | 67.1 | 10.7 | 98.2 | 99.7 |
| | 5.5 | 40° C. 1 w | 2.2 | 97.6 | 0.2 | 29.5 | 58.1 | 12.4 | 97.5 | 99.2 |
| | | 40° C. 2 w | 2.7 | 95.2 | 2.1 | 30.9 | 58.1 | 11.0 | 96.6 | 98.9 |
| | | 40° C. 4 w | 4.0 | 93.3 | 2.7 | 42.8 | 47.4 | 9.9 | 95.2 | 98.3 |
| F4 | 20 mM | T0 | 1.0 | 99.0 | ND | 22.0 | 68.1 | 10.0 | 97.8 | 99.6 |
| | Glutamic/Histidine | 40° C. 1 w | 3.1 | 96.4 | 0.5 | 30.4 | 52.9 | 16.7 | 95.5 | 96.5 |
| | pH 4.5 | 40° C. 2 w | 4.8 | 92.0 | 3.2 | 35.5 | 45.8 | 18.7 | 93.1 | 95.0 |
| | | 40° C. 4 w | 7.1 | 87.9 | 5.0 | 44.7 | 38.9 | 16.5 | 90.6 | 90.1 |
| F5 | 20 mM | T0 | 1.0 | 99.0 | ND | 22.7 | 67.2 | 10.1 | 97.9 | 99.8 |
| | Glutamic/Histidine | 40° C. 1 w | 1.6 | 98.2 | 0.2 | 25.2 | 62.4 | 12.4 | 97.2 | 98.9 |
| | pH 5.0 | 40° C. 2 w | 2.1 | 95.8 | 2.2 | 33.6 | 53.7 | 12.8 | 96.3 | 98.5 |
| | | 40° C. 4 w | 3.1 | 94.0 | 2.9 | 41.7 | 47.3 | 11.1 | 94.6 | 97.3 |
| F6 | 20 mM | T0 | 1.0 | 99.0 | ND | 22.4 | 67.9 | 9.7 | 97.5 | 99.7 |
| | Glutamic/Histidine | 40° C. 1 w | 1.6 | 98.2 | 0.2 | 25.4 | 62.4 | 12.3 | 97.5 | 99.1 |
| | pH 5.5 | 40° C. 2 w | 2.0 | 96.0 | 2.0 | 33.0 | 55.2 | 11.8 | 96.6 | 99.0 |
| | | 40° C. 4 w | 2.8 | 94.3 | 2.9 | 42.8 | 47.0 | 10.2 | 95.2 | 98.4 |
| F7 | 20 mM Arginine pH | T0 | 1.6 | 98.4 | ND | 21.7 | 68.0 | 10.3 | 97.9 | 99.7 |
| | 8.0 | 40° C. 1 w | 3.2 | 95.0 | 1.8 | 39.5 | 52.4 | 8.1 | 96.4 | 98.9 |
| | | 40° C. 2 w | 4.1 | 93.3 | 2.6 | 54.0 | 41.4 | 4.6 | 95.1 | 97.7 |
| | | 40° C. 4 w | 5.2 | 91.1 | 3.6 | 71.1 | 25.8 | 3.1 | 90.3 | 95.7 |
| F8 | 20 mM Arginine pH | T0 | 1.5 | 98.5 | 0.1 | 25.3 | 66.0 | 8.8 | 97.9 | 99.6 |
| | 8.5 | 40° C. 1 w | 3.5 | 94.2 | 2.2 | 51.4 | 41.4 | 7.2 | 95.7 | 97.3 |
| | | 40° C. 2 w | 4.4 | 92.1 | 3.4 | 69.6 | 25.5 | 4.9 | 91.0 | 94.8 |
| | | 40° C. 4 w | 6.4 | 87.7 | 5.9 | 88.7 | 9.6 | 1.7 | 85.1 | 89.3 |

### Potency

The potency was measured by the binding assay method that is carried out with following steps: Coating plate, Coating antigen (OX40-Fc), Plate blocking, incubation, Stop and read. The binding potency expressed in % refers to the percentage of the level of the anti-OX40 antibody DS binding to the antigen (OX40-Fc) after storage as compared to the level of the anti-OX40 antibody DS binding to the antigen at an initial time point. The results of potency were showed in Table 9. No obvious changes in potency were observed.

TABLE 9
Results of potency in pH/buffer screening study

| Sample Name | Time point | Binding Potency |
|---|---|---|
| 20 mM Acetate pH 4.5 | 40° C. 4 w | 92% |
| 20 mM Glutamic/Histidine pH 4.5 | 40° C. 4 w | 96% |
| 20 mM Glutamic/Histidine pH 5.0 | 40° C. 4 w | 86% |
| 20 mM Arginine pH 8.5 | 40° C. 4 w | 91% |

### Conclusions

Based on the results of pH/buffer screening study, the stability of the anti-OX40 antibody DS was better in acetate buffer and glutamin/histidine buffer at pH 5.0 and 5.5 than that in other buffers. SEC-HPLC and Caliper-SDS results showed that the aggregation and fragment were observed at pH 4.5 and 8.5. The results of the HMW in glutamic/histidine buffer was better than in acetate buffer.

iCIEF results showed the chemical change of the anti-OX40 antibody DS at pH 5.0 and 5.5 was more slowly than in other pH conditions.

Overall, a 20 mM glutamic/histidine buffer at pH 5.0 is suggested for the anti-OX40 antibody DS, and at pH 5.5 is added for backup.

### Example 4: Excipient Screening and Surfactant Strength Screening

### Purpose

This study aimed to excipient screening and surfactant strength screening for optimal protein storage.

### Material

The anti-OX40 antibody Drug Substance (DS) has a heavy chain of SEQ ID NO: 13 and a light chain of SEQ ID NO: 14.

### Experimental Method

The anti-OX40 antibody DS was dialyzed against 20 mM glutamic/histidine buffer. 40% sucrose, 44% trehalose·2H₂O, 22.5% sorbitol, 10% PS80 and 10% PS20 stock solution was compounded into the formulations, respectively (see Table 10). Each formulation was filtered through 0.22 μm PVDF membrane filters, then was filled aseptically into 2R vials (2 mL/vial). All vials were stoppered and capped immediately after filling. The vials filled with each formulation were placed under stress conditions, i.e., 40° C. incubator, freeze-thaw (−70° C. to RT) and agitation (300 rpm, 25° C.). Samples were tested at every sampling point as described in Table 11.

TABLE 10
The formulation information in excipient
screening and surfactant strength screening study
Formulation information (w/v)

| | | |
|---|---|---|
| F1 | 20 mM Glutamic/ | 8% sucrose, 0.04% PS80 |
| F2 | Histidine buffer pH 5.0 | 8.8% trehalose•2H₂0, 0.04% PS80 |
| F3 | | 4.5% sorbitol, 0.04% PS80 |
| F4 | | 8% sucrose |
| F5 | | 8% sucrose, 0.08% PS80 |
| F6 | | 8% sucrose, 0.06% PS20 |
| F7 | 20 mM Glutamic/ | 8% sucrose, 0.04% PS80 |
| | Histidine buffer pH 5.0 | |

TABLE 11
The excipient screening and surfactant
strength screening study plan

| Attributes | Condition | | T0 | Sampling Points and Assay | |
|---|---|---|---|---|---|
| Thermal | 40° | C. | X, Y, Z | 2 Weeks (2 w) | 4 Weeks (4 w) |

| | | | |
|---|---|---|---|
| | | X | X, Z, W |
| Freeze/ | −70° C. to RT | 3 Cycles | 5 Cycles |
| Thaw | | X | X, Z |
| Agitation | 300 rpm, 25° C. | 1 D | 3 D |
| | | X | X, Z |

| | | | |
|---|---|---|---|
| Control | 2-8° | C. | Backup sample |

X = Appearance, pH, protein concentration, SEC-HPLC, Caliper-SDS (R & NR), iCIEF
Y = DSC, Osmolality
Z = Sub visible particle
W = Potency(only for the selected formulation)

### Results

### DSC

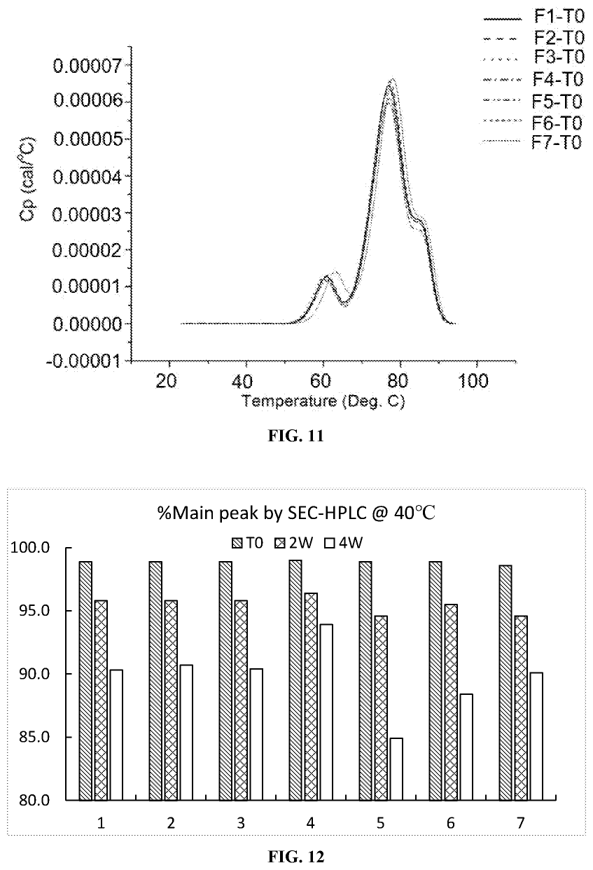

The results of DSC was showed in Table 12 and FIG. 11. The Tmonset of the anti-OX40 antibody DS in formulation F7 (53.0° C.) was higher than that in other formulations which were around 51° C.

TABLE 12
Results of DSC in excipient screening
and surfactant strength screening study

| | | | TmOnset | Tm1° | Tm2° |
|---|---|---|---|---|---|
| No | buffer | Excipients (w/v) | ° C. | C. | C. |
| F1 | 20 mM | 8% Sucrose, 0.04% PS80 | 51.4 | 61.1 | 77.0 |
| F2 | Glutamic/ | 8.8% Trehalose•2H₂0, | 51.8 | 61.1 | 77.5 |
| | Histidine | 0.04% PS80 | | | |
| F3 | pH 5.0 | 4.5% Sorbitol, 0.04% PS80 | 50.9 | 60.2 | 77.0 |
| F4 | | 8% Sucrose | 51.6 | 60.9 | 76.8 |
| F5 | | 8% Sucrose, 0.08% PS80 | 50.0 | 60.7 | 77.0 |
| F6 | | 8% Sucrose, 0.06% PS20 | 50.4 | 60.6 | 77.0 |
| F7 | 20 mM | 8% Sucrose, 0.04% PS80 | 53.0 | 63.2 | 78.3 |
| | Glutamic/ | | | | |
| | Histidine | | | | |
| | pH 5.5 | | | | |

### Appearance, pH, Protein Concentration and Osmolality

The results of appearance, pH and protein concentration was shown in Table 13. A few visible particles were observed in formulation F4 without PS82 after incubation at 40° C. for 4w, freeze-thaw and agitation, and in formulation F6 after incubation at 40° C. for 4w. No significant changes in pH and protein concentration for all the formulations under investigation. These data suggest that the surfactant is indispensable for the stability of the pharmaceutical composition of the present disclosure during, for example, freeze-thaw, storage at 40° C. for 4 weeks (where PS8 is preferred over PS20), and/or agitation for 3 days.

TABLE 13
Results of appearance, pH, protein concentration

| | Formulation | Time | | | UV280 |
|---|---|---|---|---|---|
| No | Information (w/v) | point | Appearance | pH | (mg/mL) |
| F1 | 20 mM | T0 | Slight yellow, slight | 5.0 | 52.1 |
| | Glutamic/Histidine | | opalescence, free of particles | | |
| | pH 5.0, 8% Sucrose, | 40° C. 2 W | Slight yellow, slight | 5.0 | 52.4 |
| | 0.04% PS80 | | opalescence, free of particles | | |
| | | 40° C. 4 W | Slight yellow, slight | 5.0 | 52.2 |
| | | | opalescence, free of particles | | |
| | | FT-3C* | Slight yellow, slight | 4.9 | 52.5 |
| | | | opalescence, free of particles | | |
| | | FT-5C | Slight yellow, slight | 4.9 | 52.1 |
| | | | opalescence, free of particles | | |
| | | A-1D** | Slight yellow, slight | 5.0 | 53.0 |
| | | | opalescence, free of particles | | |
| | | A-3D | Slight yellow, slight | 5.0 | 52.2 |
| | | | opalescence, free of particles | | |
| F2 | 20 mM | T0 | Slight yellow, slight | 4.9 | 52.0 |
| | Glutamic/Histidine | | opalescence, free of particles | | |
| | pH 5.0, 8.8% | 40° C. 2 W | Slight yellow, slight | 5.0 | 52.2 |
| | Trehalose·2H20, | | opalescence, free of particles | | |
| | 0.04% PS80 | 40° C. 4 W | Slight yellow, slight | 5.0 | 52.1 |
| | | | opalescence, free of particles | | |
| | | FT-3C | Slight yellow, slight | 4.9 | 52.1 |
| | | | opalescence, free of particles | | |
| | | FT-5C | Slight yellow, slight | 4.9 | 52.7 |
| | | | opalescence, free of particles | | |
| | | A-1D | Slight yellow, slight | 5.0 | 52.4 |
| | | | opalescence, free of particles | | |
| | | A-3D | Slight yellow, slight | 5.0 | 52.8 |
| | | | opalescence, free of particles | | |
| F3 | 20 mM | T0 | Slight yellow, slight | 5.0 | 52.5 |
| | Glutamic/Histidine | | opalescence, free of particles | | |
| | pH 5.0, 4.5% | 40° C. 2 W | Slight yellow, slight | 5.0 | 52.1 |
| | Sorbitol, 0.04% PS80 | | opalescence, free of particles | | |
| | | 40° C. 4 W | Slight yellow, slight | 5.0 | 52.2 |
| | | | opalescence, free of particles | | |
| | | FT-3C | Slight yellow, slight | 5.0 | 52.3 |
| | | | opalescence, free of particles | | |
| | | FT-5C | Slight yellow, slight | 4.9 | 52.2 |
| | | | opalescence, free of particles | | |
| | | A-1D | Slight yellow, slight | 5.0 | 52.5 |
| | | | opalescence, free of particles | | |
| | | A-3D | Slight yellow, slight | 5.0 | 52.7 |
| | | | opalescence, free of particles | | |
| F4 | 20 mM | T0 | Slight yellow, slight | 4.9 | 52.4 |
| | Glutamic/Histidine | | opalescence, free of particles | | |
| | pH 5.0, 8% Sucrose | 40° C. 2 W | Slight yellow, slight | 5.0 | 52.8 |
| | | | opalescence, free of particles | | |
| | | 40° C. 4 W | Slight yellow, slight | 5.0 | 52.4 |
| | | | opalescence, not essentially free | | |
| | | | of particles | | |
| | | FT-3C | Slight yellow, slight | 4.9 | 53.2 |
| | | | opalescence, not essentially free | | |
| | | | of particles | | |
| | | FT-5C | Slight yellow, slight | 5.0 | 52.5 |
| | | | opalescence, not essentially free | | |
| | | | of particles | | |
| | | A-1D | Slight yellow, slight | 5.0 | 53.1 |
| | | | opalescence, essentially free of | | |
| | | | particles | | |
| | | A-3D | Slight yellow, slight | 4.9 | 53.2 |
| | | | opalescence, not essentially free | | |
| | | | of particles | | |
| F5 | 20 mM | T0 | Slight yellow, slight | 5.0 | 51.9 |
| | Glutamic/Histidine | | opalescence, free of particles | | |
| | pH 5.0, 8% Sucrose, | 40° C. 2 W | Slight yellow, slight | 5.0 | 52.6 |
| | 0.08% PS80 | | opalescence, free of particles | | |
| | | 40° C. 4 W | Slight yellow, slight | 5.0 | 52.0 |
| | | | opalescence, free of particles | | |
| | | FT-3C | Slight yellow, slight | 4.9 | 52.3 |
| | | | opalescence, free of particles | | |
| | | FT-5C | Slight yellow, slight | 4.9 | 51.7 |
| | | | opalescence, free of particles | | |
| | | A-1D | Slight yellow, slight | 5.0 | 52.7 |
| | | | opalescence, free of particles | | |
| | | A-3D | Slight yellow, slight | 4.9 | 52.2 |
| | | | opalescence, free of particles | | |
| F6 | 20 mM | T0 | Slight yellow, slight | 5.0 | 51.8 |
| | Glutamic/Histidine | | opalescence, free of particles | | |
| | pH 5.0, 8% Sucrose, | 40° C. 2 W | Slight yellow, slight | 4.9 | 52.5 |
| | 0.06% PS20 | | opalescence, free of particles | | |
| | | 40° C. 4 W | Slight yellow, slight | 4.9 | 51.9 |
| | | | opalescence, not essentially free | | |
| | | | of particles | | |
| | | FT-3C | Slight yellow, slight | 4.9 | 52.0 |
| | | | opalescence, free of particles | | |
| | | FT-5C | Slight yellow, slight | 4.9 | 52.1 |
| | | | opalescence, free of particles | | |
| | | A-1D | Slight yellow, slight | 5.0 | 52.1 |
| | | | opalescence, free of particles | | |
| | | A-3D | Slight yellow, slight | 4.9 | 52.2 |
| | | | opalescence, free of particles | | |
| F7 | 20 mM | T0 | Slight yellow, slight | 5.5 | 50.6 |
| | Glutamic/Histidine | | opalescence, free of particles | | |
| | pH 5.5, 8% Sucrose, | 40° C. 2 W | Slight yellow, slight | 5.4 | 51.2 |
| | 0.04% PS80 | | opalescence, free of particles | | |
| | | 40° C. 4 W | Slight yellow, slight | 5.4 | 50.7 |
| | | | opalescence, free of particles | | |
| | | FT-3C | Slight yellow, slight | 5.4 | 51.1 |
| | | | opalescence, free of particles | | |
| | | FT-5C | Slight yellow, slight | 5.4 | 50.8 |
| | | | opalescence, free of particles | | |
| | | A-1D | Slight yellow, slight | 5.5 | 51.2 |
| | | | opalescence, free of particles | | |
| | | A-3D | Slight yellow, slight | 5.4 | 51.0 |
| | | | opalescence, free of particles | | |

*FT-3C means Freeze-thaw (Freeze at −70° C. and thaw at room temperature) for 3 cycles;
**A-1D means Agitation (at 300 rpm, 25° C.) for 1 day.

TABLE 14
The results of osmolality

| | Osmo- |
|---|---|
| Formulation Information (w/v) | lality |
| 20 mM Glutamic/Histidine pH 5.0, 8% Sucrose, 0.04% PS80 | 290 |
| 20 mM Glutamic/Histidine pH 5.0, 8.8% Trehalose•2H₂0, | 301 |
| 0.04% PS80 | |
| 20 mM Glutamic/Histidine pH 5.0, 4.5% Sorbitol, 0.04% PS80 | 295 |
| 20 mM Glutamic/Histidine pH 5.0, 8% Sucrose | 289 |
| 20 mM Glutamic/Histidine pH 5.0, 8% Sucrose, 0.08% PS80 | 290 |
| 20 mM Glutamic/Histidine pH 5.0, 8% Sucrose, 0.06% PS20 | 291 |
| 20 mM Glutamic/Histidine pH 5.5, 8% Sucrose, 0.04% PS80 | 293 |

### SEC-HPLC

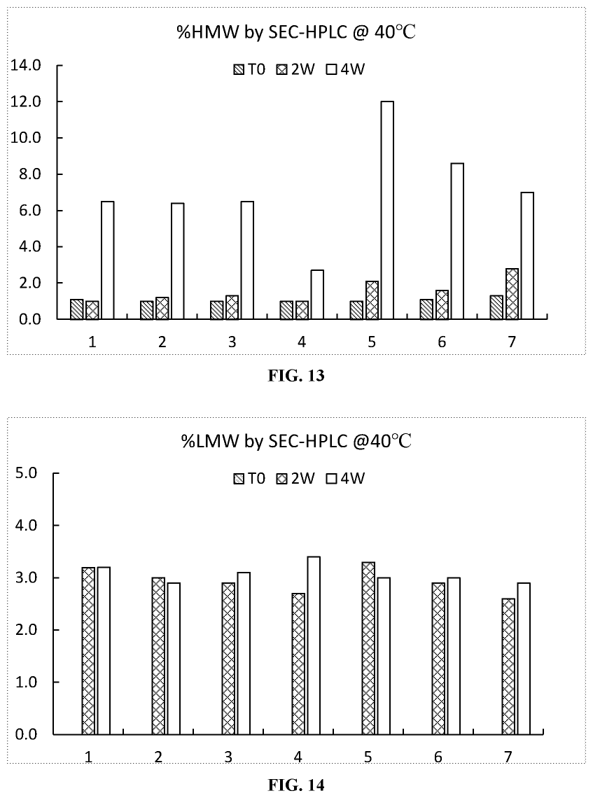

As shown in Table 15, while the compositions of main peak, aggregation and fragment were presented in FIG. 12, FIG. 13 and FIG. 14, no significant changes in SEC-HPLC were observed after freeze-thaw for 5 cycles and agitation at 25° C. for 3 days. After 4 weeks of storage at 40° C., the main peak showed the most drop by 14.0% in formulation F5 and the slightest drop by 5.1% in formulation F4.

iCIEF

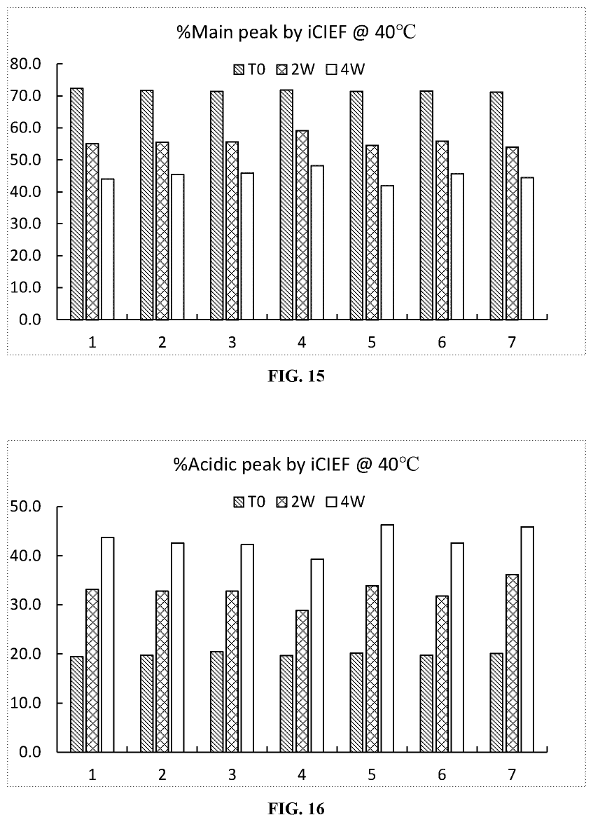

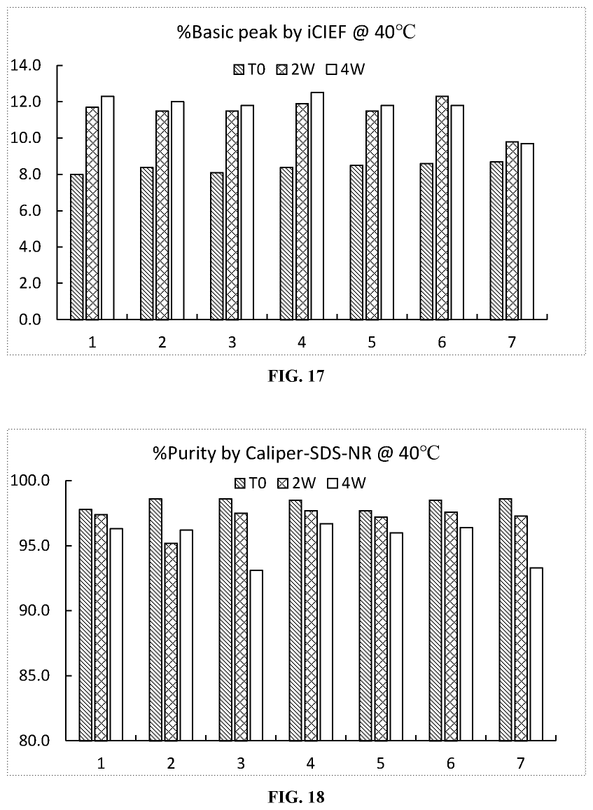

The results of the compositions of main peak, acidic peak and basic peak were shown in Table 15, FIG. 15, FIG. 16 and FIG. 17. No significant changes in iCIEF were observed after freeze-thaw for 5 cycles and agitation at 25° C. for 3 days. After 4 weeks of storage at 40° C., the results of the main peak showed the most drop by 29.5% in formulation F5, and the slightest drop by 23.7% in formulation F4.

### Caliper-SDS(N&NR)

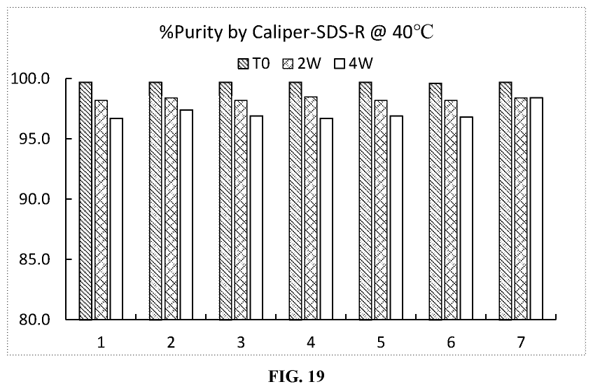

The changes in the purity of the anti-OX40 antibody DS were presented in Table 15, FIG. 18 and FIG. 19. No significant changes in Caliper-SDS(R&NR) were observed after freeze-thaw for 5 cycles and agitation at 25° C. for 3 days. After 4 weeks of storage at 40° C., the results of non-reduced in formulation F3 and F7 showed more drop by 5.5% and 5.3%, respectively. The results of reduced in formulation F7 showed the slightest drop by 1.3%, and no significant differences were observed in other formulations.

TABLE 15
Results of SEC-HPLC, iCIEF and Caliper-SDS in excipient screening and surfactant strength screening study

| | | | | | | | | | | R-Caliper- |
|---|---|---|---|---|---|---|---|---|---|---|
| | | | SEC-HPLC | | | cIEF | | | NR-Caliper- | SDS |
| | Formulation | Time | | % Main | | % Acidic | % Main | % Basic | SDS | % LC+ |
| No | Information (w/v) | point | % HMW | Peak | % LMW | Peaks | Peak | Peaks | % IgG | % HC |
| F1 | 20 mM | T0 | 1.1 | 98.9 | ND | 19.5 | 72.4 | 8.0 | 97.8 | 99.7 |
| | Glutamic/Histidine pH | 40° C. 2 W | 1.0 | 95.8 | 3.2 | 33.2 | 55.1 | 11.7 | 97.4 | 98.2 |
| | 5.0, 8% Sucrose, 0.04% | 40° C. 4 W | 6.5 | 90.3 | 3.2 | 43.7 | 44.0 | 12.3 | 96.3 | 96.7 |
| | PS80 | FT-3C | 1.0 | 98.9 | ND | 19.1 | 72.7 | 8.2 | 98.5 | 99.5 |
| | | FT-5C | 1.0 | 98.9 | ND | 19.2 | 72.4 | 8.4 | 97.4 | 99.7 |
| | | A-1D | 1.0 | 98.9 | ND | 19.5 | 71.7 | 8.8 | 98.6 | 99.7 |
| | | A-3D | 1.1 | 98.9 | ND | 19.5 | 71.5 | 9.0 | 98.5 | 99.7 |
| F2 | 20 mM | T0 | 1.0 | 98.9 | ND | 19.8 | 71.8 | 8.4 | 98.6 | 99.7 |
| | Glutamic/Histidine pH | 40° C. 2 W | 1.2 | 95.8 | 3.0 | 32.8 | 55.6 | 11.5 | 95.2 | 98.4 |
| | 5.0, 8.8% | 40° C. 4 W | 6.4 | 90.7 | 2.9 | 42.6 | 45.4 | 12.0 | 96.2 | 97.4 |
| | Trehalose•2H₂0, 0.04% | FT-3C | 1.0 | 98.9 | ND | 19.4 | 72.2 | 8.3 | 98.5 | 99.6 |
| | PS80 | FT-5C | 1.0 | 98.9 | ND | 19.6 | 72.2 | 8.2 | 98.6 | 99.6 |
| | | A-1D | 1.0 | 98.9 | ND | 20.5 | 70.7 | 8.8 | 97.8 | 99.7 |
| | | A-3D | 1.1 | 98.9 | 0.1 | 19.8 | 71.3 | 8.9 | 98.5 | 99.6 |
| F3 | 20 mM | T0 | 1.0 | 98.9 | ND | 20.5 | 71.4 | 8.1 | 98.6 | 99.7 |
| | Glutamic/Histidine pH | 40° C. 2 W | 1.3 | 95.8 | 2.9 | 32.8 | 55.7 | 11.5 | 97.5 | 98.2 |
| | 5.0, 4.5% Sorbtiol, | 40° C. 4 W | 6.5 | 90.4 | 3.1 | 42.3 | 45.9 | 11.8 | 93.1 | 96.9 |
| | 0.04% PS80 | FT-3C | 1.0 | 98.9 | ND | 19.1 | 72.8 | 8.1 | 98.4 | 99.7 |
| | | FT-5C | 1.0 | 98.9 | ND | 19.2 | 72.0 | 8.8 | 98.4 | 99.7 |
| | | A-1D | 1.0 | 99.0 | ND | 20.5 | 71.0 | 8.6 | 98.6 | 99.7 |
| | | A-3D | 1.1 | 98.9 | ND | 19.0 | 72.0 | 9.0 | 98.5 | 99.6 |
| F4 | 20 mM | T0 | 1.0 | 99.0 | ND | 19.7 | 71.9 | 8.4 | 98.5 | 99.7 |
| | Glutamic/Histidine pH | 40° C. 2 W | 1.0 | 96.4 | 2.7 | 28.9 | 59.2 | 11.9 | 97.7 | 98.5 |
| | 5.0, 8% Sucrose | 40° C. 4 W | 2.7 | 93.9 | 3.4 | 39.3 | 48.2 | 12.5 | 96.7 | 96.7 |
| | | FT-3C | 1.0 | 98.9 | ND | 19.1 | 72.4 | 8.6 | 97.5 | 99.7 |
| | | FT-5C | 1.0 | 99.0 | ND | 19.5 | 72.0 | 8.6 | 98.6 | 99.7 |
| | | A-1D | 1.0 | 99.0 | ND | 19.6 | 72.2 | 8.2 | 98.6 | 99.7 |
| | | A-3D | 1.0 | 98.9 | ND | 19.1 | 71.9 | 9.0 | 98.6 | 99.6 |
| F5 | 20 mM | T0 | 1.0 | 98.9 | ND | 20.2 | 71.4 | 8.5 | 97.7 | 99.7 |
| | Glutamic/Histidine pH | 40° C. 2 W | 2.1 | 94.6 | 3.3 | 33.9 | 54.6 | 11.5 | 97.2 | 98.2 |
| | 5.0, 8% Sucrose, 0.08% | 40° C. 4 W | 12.0 | 84.9 | 3.0 | 46.3 | 41.9 | 11.8 | 96.0 | 96.9 |
| | PS80 | FT-3C | 1.0 | 98.9 | ND | 19.5 | 71.6 | 8.9 | 98.7 | 99.6 |
| | | FT-5C | 1.0 | 98.9 | ND | 19.6 | 72.1 | 8.3 | 97.5 | 99.6 |
| | | A-1D | 1.0 | 98.9 | ND | 20.4 | 70.4 | 9.2 | 98.4 | 99.7 |
| | | A-3D | 1.1 | 98.9 | ND | 19.4 | 72.2 | 8.4 | 98.5 | 99.6 |
| F6 | 20 mM | T0 | 1.1 | 98.9 | ND | 19.8 | 71.6 | 8.6 | 98.5 | 99.6 |
| | Glutamic/Histidine pH | 40° C. 2 W | 1.6 | 95.5 | 2.9 | 31.8 | 55.9 | 12.3 | 97.6 | 98.2 |
| | 5.0, 8% Sucrose, 0.06% | 40° C. 4 W | 8.6 | 88.4 | 3.0 | 42.6 | 45.6 | 11.8 | 96.4 | 96.8 |
| | PS20 | FT-3C | 1.0 | 98.9 | ND | 19.2 | 72.0 | 8.7 | 98.6 | 99.7 |
| | | FT-5C | 1.1 | 98.9 | ND | 19.3 | 72.3 | 8.4 | 98.6 | 99.6 |
| | | A-1D | 1.1 | 98.8 | ND | 19.5 | 71.8 | 8.6 | 97.0 | 99.7 |
| | | A-3D | 1.2 | 98.8 | 0.1 | 19.3 | 71.6 | 9.1 | 98.5 | 99.7 |
| F7 | 20 mM | T0 | 1.3 | 98.6 | ND | 20.1 | 71.2 | 8.7 | 98.6 | 99.7 |
| | Glutamic/Histidine pH | 40° C. 2 W | 2.8 | 94.6 | 2.6 | 36.2 | 54.0 | 9.8 | 97.3 | 98.4 |
| | 5.5, 8% Sucrose, 0.04% | 40° C. 4 W | 7.0 | 90.1 | 2.9 | 45.9 | 44.4 | 9.7 | 93.3 | 98.4 |
| | PS80 | FT-3C | 1.3 | 98.6 | 0.1 | 19.5 | 72.1 | 8.4 | 98.8 | 99.7 |
| | | FT-5C | 1.3 | 98.7 | ND | 19.5 | 71.9 | 8.6 | 98.8 | 99.8 |
| | | A-1D | 1.3 | 98.6 | ND | 21.5 | 69.7 | 8.8 | 98.6 | 99.5 |
| | | A-3D | 1.4 | 98.5 | ND | 20.0 | 71.6 | 8.4 | 97.6 | 99.7 |

### Sub-Visible Particle

As shown in Table 16, the sub-visible particles increased in formulation F6 after incubation at 40° C. for 4 weeks. In formulation F4 without P80, the sub-visible particles increased after freeze-thaw for 5 cycles and agitation at 25° C. for 3 days

TABLE 16
Results of sub-visible particle in excipient screening
and surfactant strength screening study

| | Formulation information | Time | MFI number/mL | | | |
|---|---|---|---|---|---|---|
| No | (w/v) | point | ≥2 μm | ≥5 μm | ≥10 μm | ≥25 μm |
| F1 | 20 mM Glutamic/Histidine | T0 | 63 | 20 | 5 | 0 |
| | pH 5.0, 8% Sucrose, | 40° C. 4 W | 1488 | 451 | 48 | 4 |
| | 0.04% PS80 | FT-5C | 436 | 40 | 10 | 0 |
| | | A-3D | 37 | 7 | 0 | 0 |
| F2 | 20 mM Glutamic/Histidine | T0 | 169 | 19 | 5 | 0 |
| | pH 5.0, 8.8% | 40° C. 4 W | 875 | 210 | 69 | 0 |
| | Trehalose•2H₂0, 0.04% | FT-5C | 300 | 37 | 7 | 0 |
| | PS80 | A-3D | 35 | 10 | 5 | 0 |
| F3 | 20 mM Glutamic/Histidine | T0 | 45 | 5 | 0 | 0 |
| | pH 5.0, 4.5% Sorbtiol, | 40° C. 4 W | 783 | 112 | 20 | 0 |
| | 0.04% PS80 | FT-5C | 475 | 33 | 9 | 0 |
| | | A-3D | 112 | 10 | 4 | 0 |
| F4 | 20 mM Glutamic/Histidine | T0 | 10520 | 6960 | 3748 | 110 |
| | pH 5.0, 8% Sucrose | 40° C. 4 W | 1188 | 472 | 168 | 9 |
| | | FT-5C | 11907 | 7109 | 3653 | 166 |
| | | A-3D | 8940 | 4966 | 2203 | 143 |
| F5 | 20 mM Glutamic/Histidine | T0 | 73 | 15 | 5 | 0 |
| | pH 5.0, 8% Sucrose, | 40° C. 4 W | 629 | 164 | 48 | 9 |
| | 0.08% PS80 | FT-5C | 590 | 45 | 9 | 0 |
| | | A-3D | 69 | 15 | 10 | 5 |
| F6 | 20 mM Glutamic/Histidine | T0 | 86 | 9 | 4 | 2 |
| | pH 5.0, 8% Sucrose, | 40° C. 4 W | 161368 | 41425 | 9624 | 204 |
| | 0.06% PS20 | FT-5C | 340 | 43 | 9 | 0 |
| | | A-3D | 133 | 35 | 9 | 2 |
| F7 | 20 mM Glutamic/Histidine | T0 | 153 | 19 | 4 | 0 |
| | pH 5.5, 8% Sucrose, | 40° C. 4 W | 731 | 84 | 5 | 0 |
| | 0.04% PS80 | FT-5C | 421 | 43 | 9 | 2 |
| | | A-3D | 68 | 17 | 0 | 0 |

### Potency

As shown in Table 17, no significant changes in potency were observed after incubation at 40° C. for 4 weeks.

TABLE 17
Results of the potency in excipient screening
and surfactant strength screening study

| Sample Name | Time point | Binding Potency |
|---|---|---|
| 20 mM Glutamic/Histidine pH 5.0, | 40° C. 4 W | 96% |
| 8% Sucrose, 0.04% PS80 | | |
| 20 mM Glutamic/Histidine pH 5.0, | 40° C. 4 W | 90% |
| 8% Sucrose, 0.08% PS80 | | |

### kD and Tagg

### Purpose

Base on the results of excipient screening and surfactant strength screening study, the kD and Tagg investigated the diffusion and aggregation of the protein with different surfactant strength.

### Experimental Method

The spare sample of formulation F4 (20 mM glutamic/histidine pH 5.0, 8% (w/v) Sucrose) was filtered through 0.22 μm PVDF membrane filters and diluted to different protein concentration by formulation buffer. 10% PS80 stock solution was compounded into the formulations. The protein concentration and surfactant strength was showed in Table 18. Only 2 mg/mL, 6 mg/mL, 10 mg/mL were studied by Tagg. The temperature of kD was 20° C., 25° C., 30° C., 35° C. and 40° C.

TABLE 18
The protein concentration and surfactant
strength in the study of kD and Tagg

| | |
|---|---|
| PS80 | Protein concentration mg/mL |

| | | | | | | |
|---|---|---|---|---|---|---|
| concentration % | 1 | 2 | 4 | 6 | 8 | 10 |

| | |
|---|---|
| 0 | kD/Tagg |

0.01
0.02
0.04

### kD Results

As shown in Table 19 Results of kD in formulation with 0.04% PS80 was worse than in other formulations.

TABLE 19
Results of kD
kD

| Surfactant | Temperature ° C. | | | | |
|---|---|---|---|---|---|
| strength | 20 | 25 | 30 | 35 | 40 |
| 0.01% PS80 | 10.7 | 13.1 | 12.2 | 17.4 | 16.8 |
| 0.02% PS80 | 14.1 | 18.7 | 12.5 | 19.2 | 19.0 |
| 0.04% PS80 | 9.1 | 10.4 | 13.3 | 11.8 | 15.4 |
| PS80 Free | 13.3 | 15.5 | 14.0 | 21.8 | 25.1 |

### Tagg Results

As shown in Table 20, no significant differences were observed in all formulations.

TABLE 20
Results of Tagg

| Sample information | DLS Temp ° C. |
|---|---|
| PS80 Free 2 mg/mL | 61.49 |
| PS80 Free 6 mg/mL | 60.24 |
| PS80 Free 10 mg/mL | 59.23 |
| 0.01% PS80 2 mg/mL | 59.38 |
| 0.01% PS80 6 mg/mL | 60.27 |
| 0.01% PS80 10 mg/mL | 59.42 |
| 0.01% PS80 2 mg/mL | 61.70 |
| 0.01% PS80 6 mg/mL | 60.61 |
| 0.01% PS80 10 mg/mL | 59.87 |
| 0.01% PS80 2 mg/mL | 61.59 |
| 0.01% PS80 6 mg/mL | 60.65 |
| 0.01% PS80 10 mg/mL | 60.14 |

### Conclusions

The stability of the anti-OX40 antibody DS was investigated after incubation at 40° C. for 4 weeks, freeze-thaw for 5 cycles and agitation at 300 rpm in different formulation conditions containing sucrose, trehalose, sorbitol, PS80, PS20 and different surfactant strength, respectively.

The results showed no significant discrepancy between sucrose, trehalose, and sorbitol in stabilizing proteins. The visible particles were observed in formulation with PS20 after incubation at 40° C. for 4 weeks. The main peak of SEC-HPLC showed the most drop by 14.0% in formulation with 0.08% PS80, it means higher surfactant strength was not good for protein conformation.

The results of kD showed that the diffusion of the anti-OX40 antibody DS in formulation with 0.04% PS80 was weaker, yet still acceptable, than in formulation with 0.01% PS80 and 0.02% PS80 or without PS80.

Therefore, two formulations which were 20 mM glutamic/histidine buffer pH 5.0, 8% (w/v) sucrose, 0.02% (w/v) PS80 and 20 mM glutamic/histidine buffer pH 5.0, 8% (w/v) sucrose, 0.04% (w/v) PS80 were recommended as the formulation for the anti-OX40 antibody DS in formulation confirmation study.

The optimized pharmaceutical formulations are summarized in Table 21 below.

TABLE 21
the optimized pharmaceutical formulations

| | Concentration | | Preferred | | |
|---|---|---|---|---|---|
| Ingredient | Ranges | | Concentration | | Function |
| the anti-OX40 antibody DS | 40-60 | mg/mL | 50 | mg/mL | API |
| Glutamic/histidine buffer | 10-30 | mmol/L | 20 | mmol/L | Buffer |
| pH 5.0*** | | | | | |
| Sucrose | 4-12% | (w/v) | 8% | (w/v) | Stabilizer |
| Polysorbate 80 | 0.01-0.05% | (w/v) | 0.02% | (w/v) | Surfactant |
| Water for injection | | | | | Diluent |

Note:
***20 mmol/L Glutamic/histidine buffer with pH of 5.0 comprises 9.39 mmol/L glutamic acid and 10.61 mmol/L histidine.

### Example 5: Long-Term Stability Study for the Pharmaceutical Formulations

Two batches of pharmaceutical formulation of the present disclosure (Non-GMP Batch No. 1 and GMP Batch No. 2, see Table 22) were prepared using the anti-OX40 antibody DS, a glutamic/histidine buffer (prepared from 9.39 mmol/L glutamic acid and 10.61 mmol/L histidine), sucrose and polysorbate 80, wherein the concentration of the anti-OX40 antibody DS was 50 mg/ml, the concentration of the glutamic/histidine buffer was 20 mmol/L, the concentration of sucrose was 8% (w/v), the concentration of polysorbate 80 was 0.02% (w/v) and the pH of the formulation was about 5.0. Long-term stability tests are performed on the two batches by storing the two batches of pharmaceutical formulation under the storage conditions as shown in Table 23 and samples were periodically taken to measure the stability of the pharmaceutical formulation. The quality attributes monitored included color, clarity, pH, polysorbate 80 content, visible particle, subvisible particulate matter, CEX-HPLC, SEC-UPLC, CE-SDS (reduced and non-reduced), protein concentration, binding potency, sterility and container closure integrity test (CCIT). CCIT was performed at annual time points and at expiry at the long-term storage condition.

TABLE 22
Pharmaceutical Formulation Batch Information

| Batch | | |
|---|---|---|
| No. | Pharmaceutical Formulation | Container closure system |
| 1 | 50 mg/mL anti-OX40 antibody DS, | 2 R Type I glass vial with |
| 2 | 20 mM L-glutamic acid/L-histidine | a 13 mm rubber stopper |
| | buffer, 8% (w/v) sucrose and 0.02% | integrated into a plastic |
| | (w/v) polysorbate 80, pH 5.0. | cap |

TABLE 23
Storage Conditions and Testing Time Points

| | Storage | |
|---|---|---|
| Batch No. | condition | Time points ¹ |
| 1 | 5 ± 3° C. | T0, 1 M, 3 M, 6 M, 9 M, 12 M, 18 M, 24 M |
| 2 | | |

Note:
¹M = month

3-month stability data for Non-GMIP Batch No. 1 and GMIP Batch No. 2 are shown in Table 24 and Table 25. No trends or significant changes were observed for the two batches at the long-term storage condition.

TABLE 24
Long-term Stability Data for Non-GMP Batch No. 1 at 5 ± 3° C.

| Attribute | Test (Units) | | T0 | 1 Month | 3 Month |
|---|---|---|---|---|---|
| General | Color | | <Y2³ | <Y2 | <Y2 |
| | Clarity (NTU) | | 7.2 | 7.7 | 7.7 |
| | pH | | 5.1 | 5.1 | 5.1 |
| | Polysorbate 80 content (%, w/v) | | 0.019 | NT | NT |
| | Visible particle | | No visible | No visible | No visible |
| | | | particles | particles | particles |
| | | | present | present | present |
| | Subvisible | ≥2 μm | 819 | 1159 | 766 |
| | particulate | (particles/container) | | | |
| | matter | ≥5 μm | 153 | 220 | 101 |
| | | (particles/container) | | | |
| | | ≥10 μm | 18 | 21 | 12 |
| | | (particles/container) | | | |
| | | ≥25 μm | 0 | 0 | 0 |
| | | (particles/container) | | | |
| Purity | CEX-HPLC | Main peak (%) | 76.3 | 76.2 | 76.5 |
| | | Acidic peaks (%) | 14.2 | 14.7 | 14.5 |
| | | Basic peaks (%) | 9.5 | 9.2 | 9.0 |
| | SEC-UPLC | Main peak (%) | 99.3 | 99.3 | 98.6 |
| | | HMWS (%) | 0.6 | 0.6 | 0.8 |
| | | LMWS (%) | 0.1 | 0.1 | 0.6 |
| | CE-SDS | (LC + HC) (%) | 97.8 | 97.5 | 97.2 |
| | (Reduced) | Total LMWS (%) | 1.1 | 1.7 | 2.0 |
| | CE-SDS | Main peak (%) | 97.7 | 97.7 | 97.9 |
| | (Non-reduced) | Total LMWS (%) | 2.1 | 2.2 | 2.1 |

| | | | | |
|---|---|---|---|---|
| Quantity | Protein concentration (mg/mL) | 50.0 | 50.7 | 50.6 |
| Potency | Binding potency | 94 | 93 | 91 |
| | (% relative potency) | | | |
| Safety¹ | Sterility | No growth | NT | NT |
| | CCIT | NT² | NT | NT |

Note:
¹Sterility was performed for release and at expiry, and CCIT is performed at annual time points and at expiry.
²“NT” indicates that the assay was not performed for the indicated time points.
³“Y2” indicates the standard for yellow solutions of color testing, as defined in Chinese Pharmacopoeia (2020 edition). The standard for yellow solutions is Y1 to Y10. The smaller number is, the lighter the color. “<Y2” indicates that the color change is less than the Y2 standard solution for the formulated anti-OX40 monoclonal antibody drug product.

TABLE 25
Long-term Stability Data for GMP Batch No. 2 at 5 ± 3° C.

| | | | Acceptance | | | |
|---|---|---|---|---|---|---|
| Attribute | Test (Units) | | criteria | T0 | 1 Month | 3 Month |
| General | Color | | Not more | <Y2³ | <Y2 | <Y2 |
| | | | colored | | | |
| | | | than No. 3 | | | |
| | | | standard | | | |
| | | | solution | | | |
| | Clarity (NTU) | | ≤30.0 | 7.2 | 6.5 | 6.7 |
| | pH | | 4.7-5.3 | 5.1 | 5.1 | 5.1 |
| | Polysorbate 80 | | Report | 0.021 | NT | NT |
| | content (%, w/v) | | result | | | |
| | Visible particle | | Liquid, | Liquid, | Liquid, | Liquid, |
| | | | essentially | essentially | essentially | essentially |
| | | | free of | free of | free of | free of |
| | | | visible | visible | visible | visible |
| | | | particles | particles | particles | particles |
| | Subvisible | ≥2 μm | Report | 558 | 945 | 1120 |
| | particulate | (particles/ | result | | | |
| | matter | container) | | | | |
| | | ≥5 μm | Report | 76 | 158 | 156 |
| | | (particles/ | result | | | |
| | | container) | | | | |
| | | ≥10 μm | ≤6000 | 7 | 12 | 5 |
| | | (particles/ | | | | |
| | | container) | | | | |
| | | ≥25 μm | ≤600 | 0 | 0 | 0 |
| | | (particles/ | | | | |
| | | container) | | | | |
| Purity | CEX- | Main peak (%) | ≥40.0 | 78.8 | 76.3 | 78.9 |
| | HPLC | Acidic peaks (%) | ≤40.0 | 12.6 | 13.7 | 12.3 |
| | | Basic peaks (%) | ≤30.0 | 8.5 | 10.0 | 8.7 |
| | SEC- | Main peak (%) | ≥95.0 | 99.4 | 99.0 | 99.1 |
| | UPLC | HMWS (%) | ≤5.0 | 0.6 | 0.6 | 0.7 |
| | | LMWS (%) | Report | 0.1 | 0.4 | 0.2 |
| | | | result | | | |
| | CE-SDS | (LC + HC) (%) | ≥90.0 | 97.9 | 98.0 | 98.5 |
| | (Reduced) | Total LMWS (%) | Report | 1.3 | 1.2 | 0.9 |
| | | | result | | | |
| | CE-SDS | Main peak (%) | ≥90.0 | 97.7 | 97.8 | 97.7 |
| | (Non-reduced) | Total LMWS (%) | Report | 2.1 | 2.0 | 2.0 |
| | | | result | | | |

| | | | | | |
|---|---|---|---|---|---|
| Quantity | Protein concentration (mg/mL) | 45.0-55.0 | 50.2 | 50.0 | 49.8 |
| Potency | Binding potency (% | 60-140 | 103 | 97 | 105 |
| | relative potency) | | | | |
| Safety¹ | Sterility | No | No | NT | NT |
| | | growth | growth | | |
| | CCIT | Pass | NT2 | NT | NT |

Note:
¹Sterility was performed for release and at expiry, and CCIT is performed at annual time points and at expiry.
²“NT” indicates that the assay was not performed for the indicated time points.
³“Y2” indicates a standard for yellow solutions of color testing, as defined in Chinese Pharmacopoeia (2020 edition). The standard for yellow solutions is Y1 to Y10. The smaller number is, the lighter the color. “<Y2” indicates that the color change is less than the Y2 standard solution for the formulated anti-OX40 monoclonal antibody drug product.

### Example 6: Animal Study for Toxicity and Toxicokinetics

### Purpose

The purpose of this study is to determine the potential toxicity of the pharmaceutical formulation provided herein when administered by intravenous (IV) infusion to cynomolgus monkey over 29 days on Days 1, 8, 15, 22 and 29. Also to assess the reversibility, persistence, or delayed occurrence of toxic effects following a 28-Day recovery period. In addition, the toxicokinetics (TK), immunogenicity, and safety pharmacology evaluation of the pharmaceutical formulation provided herein is determined.

### Experimental Design

Forty (20/sex) cynomolgus monkeys are randomly assigned to 4 groups of 5/sex/group to determine the toxicity of the pharmaceutical formulation as shown in Table 26 after once weekly intravenous (IV) infusion on Days 1, 8, 15, 22 and 29. The control group are administered with vehicle. Animals are randomly assigned to groups by Provantis based on body weight. The study design is shown in Table 27.

TABLE 26
the tested pharmaceutical formulations

| Ingredient | Concentration | | Function |
|---|---|---|---|
| the anti-OX40 antibody DS | 50 | mg/mL | API |
| Glutamic/histidine buffer pH | 20 | mmol/L | Buffer |
| 5.0*** | | | |
| Sucrose | 8% | (w/v) | Stabilizer |
| Polysorbate 80 | 0.02% | (w/v) | Surfactant |
| Water for injection | | | Diluent |

Note:
***20 mmol/L Glutamic/histidine buffer with pH of 5.0 comprises 9.39 mmol/L glutamic acid and 10.61 mmol/L histidine.

The last 2 monkeys/sex/group is allocated for recovery.

All available dosing-phase animals in Group 1 to 4 are necropsied on Day 30. All available recovery animals in Group 1 to 4 are necropsied on Day 58.

TABLE 27
Study Design

| | | the anti-OX40 antibody | | | | | | |
|---|---|---|---|---|---|---|---|---|
| | | DS Doses^{a} | | | Numbering of Animals^{b} | | | |
| Group | Group | Dose | Volume | Conc. | Dosing Phase | | Recovery | |
| Numbers | Designation | (mg/kg/day) | (mL/kg) | (mg/mL) | M | F | M | F |
| 1 | Control | 0 | 5 | 0 | 1001- | 1501- | 1004- | 1504- |
| | | | | | 1003 | 1503 | 1005 | 1505 |
| 2 | Low Dose | 30 | 5 | 6 | 2001- | 2501- | 2004- | 2504- |
| | | | | | 2003 | 2503 | 2005 | 2505 |
| 3 | Mid Dose | 100 | 5 | 20 | 3001- | 3501- | 3004- | 3504- |
| | | | | | 3003 | 3503 | 3005 | 3505 |
| 4 | High Dose | 200 | 5 | 40 | 4001- | 4501- | 4004- | 4504- |
| | | | | | 4003 | 4503 | 4005 | 4505 |

Note:
In this protocol, “dose level” and “dosage” are used interchangeably. Conc. = Concentration; M = Male; F = Female.
^{a}indicates doses of active pharmaceutical ingredient unless specified otherwise.
^{b}indicates that replacement animals, if any, are numbered per Testing Facility SOP and included in the study report.

### Criteria for Evaluation

Criteria for evaluation included viability (morbidity/mortality), clinical observations, body weight, food consumption, clinical pathology (hematology, serum chemistry, coagulation, urinalyses), body temperature, organ weight, gross (necropsy) evaluation, histopathological evaluation, immunogenicity/immunotoxicology evaluation and toxicokinetics.

1. A pharmaceutical formulation, comprising a monoclonal anti-OX40 antibody or antigen-binding fragment thereof, a buffer, a stabilizer, and a surfactant.

2. The pharmaceutical formulation of claim 1, wherein the monoclonal anti-OX40 antibody comprises a heavy chain and a light chain, wherein the heavy chain comprises a heavy chain variable region VH which comprises:
HCDR1 comprising the amino acid sequence as set forth in SEQ ID NO: 1;
HCDR2 comprising the amino acid sequence as set forth in SEQ ID NO: 2,
HCDR3 comprising the amino acid sequence as set forth in SEQ ID NO: 3;
wherein the light chain comprises a light chain variable region VL which comprises:
LCDR1 comprising the amino acid sequence as set forth in SEQ ID NO: 4,
LCDR2 comprising the amino acid sequence as set forth in SEQ ID NO: 5,
LCDR3 comprising the amino acid sequence as set forth in SEQ ID NO: 6; and
wherein the heavy chain further comprises Fc region variant, and the Fc region variant is human IgG1 N297A.

3. The pharmaceutical formulation of claim 2, wherein the heavy chain variable region VH comprises the amino acid sequence selected from the group consisting of: SEQ ID NO: 7, SEQ ID NO: 9, and SEQ ID NO: 10.

4. The pharmaceutical formulation of claim 2, wherein the light chain variable region VL comprises the amino acid sequence selected from the group consisting of: SEQ ID NO: 8, SEQ ID NO: 11, and SEQ ID NO: 12.

5. The pharmaceutical formulation of claim 1, wherein the concentration of the monoclonal anti-OX40 antibody in the pharmaceutical formulation is about 0.5-200 mg/ml, preferably about 40-60 mg/ml.

6. The pharmaceutical formulation of claim 1, wherein the pharmaceutical formulation has a pH of about 5.0-8.0.

7. The pharmaceutical formulation of claim 1, wherein the buffer is selected from the group consisting of acetate buffer, histidine buffer, citrate buffer, glutamic acid buffer, arginine buffer, citrate & arginine buffer, and glutamic acid & histidine buffer, aspartic acid & histidine buffer, and the concentration of the buffer in the pharmaceutical formulation is about 1-100 mmol/L.

8. The pharmaceutical formulation of claim 1, wherein the stabilizer is selected from the group consisting of sucrose, sorbitol, trehalose, xylitol and mannose, and the concentration of the stabilizer in the pharmaceutical formulation is about 0.5%-50% w/v.

9. The pharmaceutical formulation of claim 1, wherein the surfactant is selected from the group consisting of polysorbate 80 and polysorbate 20, and the concentration of the surfactant in the pharmaceutical formulation is about 0.001-0.1% w/v.

10. The pharmaceutical formulation of claim 1, wherein the concentration of the monoclonal anti-OX40 antibody in the pharmaceutical formulation is about 40-60 mg/ml, the concentration of the buffer in the pharmaceutical formulation is about 10-30 mmol/L, the concentration of the stabilizer in the pharmaceutical formulation is about 4-12% w/v, the concentration of the surfactant in the pharmaceutical formulation is about 0.01-0.05% w/v, and/or the pharmaceutical formulation has a pH of about 5.0-6.0.

11. The pharmaceutical formulation of claim 1, wherein the concentration of the monoclonal anti-OX40 antibody in the pharmaceutical formulation is about 50 mg/ml, the concentration of the buffer in the pharmaceutical formulation is about 20 mmol/L, the concentration of the stabilizer in the pharmaceutical formulation is about 4.5-8.8% w/v, the concentration of the surfactant in the pharmaceutical formulation is about 0.02-0.04% w/v, and/or wherein the pharmaceutical formulation has a pH of about 5.0-5.5.

12. The pharmaceutical formulation of claim 11, wherein the concentration of the stabilizer in the pharmaceutical formulation is about 8% w/v, the concentration of the surfactant in the pharmaceutical formulation is about 0.02% w/v, and/or the pharmaceutical formulation has a pH of about 5.0.

13. The pharmaceutical formulation of claim 1, wherein the buffer is glutamic acid & histidine buffer, aspartic acid & histidine buffer or combination thereof; and/or
wherein the stabilizer is sucrose, sorbitol, trehalose, or combination thereof; and/or
wherein the surfactant is polysorbate 80.

14. The pharmaceutical formulation of claim 1, comprising:
a monoclonal anti-OX40 antibody or antigen-binding fragment thereof, at a concentration of about 40-60 mg/ml,
glutamic acid & histidine buffer or aspartic acid & histidine buffer at a concentration of about 10-30 mmol/L,
sucrose at a concentration of about 4-12% w/v, and
polysorbate 80 at a concentration of about 0.01-0.05% w/v,
and the pharmaceutical formulation has a pH of about 5.0-5.5.

15. The pharmaceutical formulation of claim 1, comprising:
a monoclonal anti-OX40 antibody or antigen-binding fragment thereof, at a concentration of about 50 mg/ml,
glutamic acid & histidine buffer or aspartic acid & histidine buffer at a concentration of about 20 mmol/L,
sucrose at a concentration of about 8% w/v, and
polysorbate 80 at a concentration of about 0.02% w/v,
and wherein the pharmaceutical formulation has a pH of about 5.0.

16. The pharmaceutical formulation of claim 1, wherein the pharmaceutical formulation is suitable for subcutaneous administration or for intravenous administration.

17-18. (canceled)

19. A method of treating OX40-associated disease in a subject in need thereof, comprising administering a therapeutically effective amount of the pharmaceutical formulation of claim 1 to the subject.

20. The method of claim 19, wherein the administration is via subcutaneous injection or intravenous injection.

21. (canceled)

22. A kit comprising the pharmaceutical formulation of claim 1 in one or more containers.

23. The kit of claim 22, further comprises instructions for use of the kit.
